(19) United States
(12) Patent Application Publication
(10) Pub. No.: US 2003/0073836 A1
(43) Pub. Date: Apr. 17, 2003

(54) Heteroarylcarboxylic acid amides, the preparation thereof and their use as pharmaceutical compositions

(75) Inventors: Henning Priepke, Warthausen (DE); Norbert Hauel, Schemmerhofen (DE); Georg Dahmann, Attenweiler (DE); Leo Thomas, Biberach (DE); Michael Mark, Biberach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma KG, Ingelheim (DE)

(21) Appl. No.: 10/187,860

(22) Filed: Jul. 2, 2002

Related U.S. Application Data
(60) Provisional application No. 60/304,584, filed on Jul. 11, 2001.

(30) Foreign Application Priority Data
Jul. 11, 2001 (DE) DE 101 32 686.6

(51) Int. Cl. C 07D 4 3/02; C07D 207/00

(52) U.S. Cl. 544/238; 544/333; 544/405; 546/268.1; 548/530

(57) ABSTRACT

A compound of formula

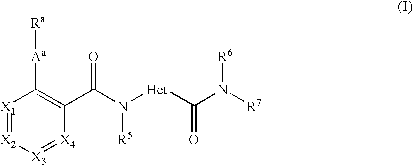

wherein: A^{a}, R^{a}, X₁ to X₄, Het, and R⁵ to R⁷ are defined as in claim 1, the isomers and the salts thereof, particularly the physiologically acceptable salts thereof, which are valuable inhibitors of the microsomal triglyceride-transfer protein (MTP), medicaments containing these compounds and their use, as well as the preparation thereof.

### RELATED APPLICATIONS

Benefit under 35 U.S.C. §119(e) of prior U.S. provisional application Serial No. 60/304,584, filed Jul. 11, 2001, is hereby claimed.

### SUMMARY OF THE INVENTION

The present invention relates to heteroarylcarboxylic acid amides of general formula

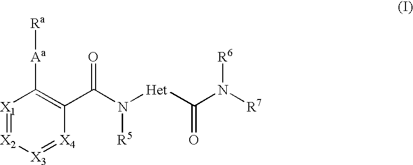

their tautomers, their diastereomers, their enantiomers, the mixtures and the salts thereof, particularly the physiologically acceptable salts thereof which have valuable pharmacological properties, pharmaceutical compositions containing these compounds, the use thereof and processes for the preparation thereof.

The compounds of the above general formula I are valuable inhibitors of the microsomal triglyceride-transfer protein (MTP) and are therefore suitable for lowering the plasma level of the atherogenic lipoproteins.

In the above general formula I

X₁ denotes the group CR¹;

X₂ denotes the group CR²;

X₃ denotes the group CR³; and

X₄ denotes the group CR⁴, or

one or two of the groups X₁ to X₄ in each case denote a nitrogen atom, and the remainder of the groups X₁ to X₄ denote three or two of the groups CR¹ to CR⁴,

while R¹, R², R³, and R⁴ in each case denote a hydrogen atom or

one or two of the groups R¹ to R⁴ independently of one another in each case denote a fluorine, chlorine, or bromine atom, a C₁₋₃-alkyl group, a trifluoromethyl, hydroxy, C₁₋₃-alkoxy, trifluoromethoxy, amino, C₁₋₃-alkylamino, or di-(C₁₋₃-alkyl)-amino group, and the remainder of the groups R¹ to R⁴ in each case represent a hydrogen atom,

while R⁴ additionally together with R⁵ may assume the meaning of a-(CH₂)ₙ— bridge wherein n denotes the number 1, 2, or 3, and

A^{a} denotes a bond, an oxygen or sulfur atom, an —NH, —N(C₁₋₃-alkyl), sulfinyl, sulfonyl, or carbonyl group,

one of the groups —CH₂—, —(CH₂)₂—, —CH═CH—, —C≡C—, —OCH₂—, —CH₂O—, —NH—CH₂—, —CH₂—NH—, —NH—CO—, —CO—NH—, —NH—SO₂—, or —SO₂—NH—,

wherein a hydrogen atom bound to a carbon atom and/or a hydrogen atom bound to a nitrogen atom may be replaced in each case by a C₁₋₃-alkyl group, and wherein a heteroatom of the group A^{a} is not linked to a nitrogen atom of a 5-membered heteroaryl group of the group R^{a},

R^{a} denotes a phenyl, 1-naphthyl, or 2-naphthyl group,

a 5-membered heteroaryl group bound via a carbon or nitrogen atom, which contains

an imino group optionally substituted by a C₁₋₄-alkyl or C₁₋₄-alkyl-carbonyl group, an oxygen or sulfur atom,

an imino group optionally substituted by a C₁₋₄-alkyl group or an oxygen or sulfur atom and additionally a nitrogen atom, or

an imino group optionally substituted by a C₁₋₄-alkyl group and two nitrogen atoms, or

an oxygen or sulfur atom and two nitrogen atoms,

a 6-membered heteroaryl group which contains one or two nitrogen atoms,

while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety, and

wherein the abovementioned phenyl and naphthyl groups as well as the mono- and bicyclic heteroaryl groups in the carbon skeleton may be monosubstituted by a fluorine, chlorine, or bromine atom, or by a C₁₋₄-alkyl, C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)amino, acetylamino, N-(C₁₋₃-alkyl)-acetylamino, propionylamino, N-(C₁₋₃-alkyl)-propionylamino, acetyl, propionyl, C₁₋₃-alkoxy-carbonyl, aminocarbonyl, C₁₋₃-alkylaminocarbonyl, di-(C₁₋₃-alkyl)aminocarbonyl, or cyano group or, with the exception of 5-membered heteroaryl groups containing more than two heteroatoms, may also be disubstituted by the abovementioned substituents, while the substituents may be identical or different,

a C₃₋₇-cycloalkyl group, wherein

in each case the methylene group in the 4 position of a 6- or 7-membered cycloalkyl group may be replaced by an oxygen or sulfur atom, by a sulfinyl or sulfonyl group, or by an imino group optionally substituted by a C₁₋₅-alkyl, phenyl, C₁₋₄-alkyl-carbonyl, C₁₋₄-alkoxy-carbonyl, C₁₋₃-alkylaminocarbonyl, or di-(C₁₋₃-alkyl)-aminocarbonyl group,

a 4- to 7-membered cycloalkyleneimino group wherein

the cycloalkylene moiety may be fused to a phenyl ring, or

one or two hydrogen atoms may be replaced in each case by a C₁₋₃-alkyl group and/or

in each case the methylene group in the 4 position of a 6- or 7-membered cycloalkyleneimino group may be substituted by a hydroxycarbonyl, C₁₋₃-alkoxy-carbonyl, amino, C₁₋₃-alkylamino, di-(C₁₋₃-alkyl)amino, aminocarbonyl, C₁₋₃-alkylaminocarbonyl, di-(C₁₋₃-alkyl)-aminocarbonyl, or phenyl-C₁₋₃-alkylamino group, or

may be replaced by an oxygen or sulfur atom, by a sulfinyl or sulfonyl group, or by an imino group optionally substituted by a C₁₋₅-alkyl, phenyl, C₁₋₄-alkyl-carbonyl, C₁₋₄-alkoxy-carbonyl, C₁₋₃-alkylaminocarbonyl, or di-(C₁₋₃-alkyl)-aminocarbonyl group or

in a 5-, 6-, or 7-membered cycloalkyleneimino group a-CH₂— group linked to the imino nitrogen atom may be replaced by a carbonyl group, or a —(CH₂)₂— group linked to the imino nitrogen atom may be replaced by a —CO—NR⁸— group, or a —(CH₂)₃— group linked to the imino nitrogen atom may be replaced by a —CO—NR⁸—CO— group,

while R⁸ denotes a hydrogen atom or a C₁₋₃-alkyl group,

R⁵ denotes a hydrogen atom or a C₁₋₅-alkyl group,

Het denotes a 5-membered heteroarylene group bound via two carbon atoms or, if Het denotes a double-bonded pyrrole group, it may also be bound via a carbon atom and the imino-nitrogen atom, the latter being linked to the adjacent carbonyl group in formula I, which contains

an imino group substituted by the group R⁹, an oxygen or sulfur atom, or

an imino group substituted by the group R⁹ or an oxygen or sulfur atom and additionally a nitrogen atom,

while R⁹ denotes a hydrogen atom, a C₁₋₅-alkyl group, a C₂₋₃-alkyl group terminally substituted by an amino, C₁₋₃-alkylamino, di-(C₁₋₃-alkyl)-amino, or CIl5-alkoxy-carbonylamino group, a carboxy-C₁₋₃-alkyl, C₁₋₃-alkoxy-carbonyl-C₁₋₃-alkyl, phenyl, phenyl-C₁₋₃-alkyl, C₁₋₅-alkyl-carbonyl, or phenylcarbonyl group, or R⁹ together with R⁶ denotes a —(CH₂)ₚ— bridge, wherein p denotes the number 2 or 3,

or an imino group optionally substituted by a C₁₋₃-alkyl group and two nitrogen atoms, or

an oxygen or sulfur atom and two nitrogen atoms,

or a 6-membered heteroarylene group which contains one or two nitrogen atoms,

while the abovementioned heteroarylene groups in the carbon skeleton may be monosubstituted by a fluorine, chlorine, or bromine atom, by a C₁₋₅-alkyl group, by a C₃₋₇-cycloalkyl, trifluoromethyl, hydroxy, C₁₋₃-alkoxy, trifluoromethoxy, amino, C₁₋₃-alkylamino, di-(C₁₋₃-alkyl)amino, acetylamino, N-(C₁₋₃-alkyl)-acetylamino, propionylamino, N-(C₁₋₃-alkyl)-propionylamino, acetyl, propionyl, benzoyl, C₁₋₃-alkoxy-carbonyl, aminocarbonyl, C₁₋₃-alkylaminocarbonyl-, di-(C₁₋₃-alkyl)aminocarbonyl, or cyano group or, with the exception of 5-membered monocyclic heteroaryl groups containing more than one heteroatom, may also be disubstituted by the abovementioned substituents, while the substituents may be identical or different,

R⁶ denotes a hydrogen atom or a C₁₋₆-alkyl group,

R⁷ denotes a C₁₋₉-alkyl group,

a straight-chain or branched, mono-, di-, or triunsaturated C₃₋₉-alkenyl or C₃₋₉-alkynyl group, while the multiple bonds are isolated from the nitrogen-carbon bond,

a straight-chain C₂₋₆-alkyl group which is terminally substituted by an amino, C₁₋₃-alkylamino, or di-(C₁₋₃-alkyl)-amino group,

a C₁₋₆-alkyl group substituted by a C₃₋₇-cycloalkyl group, while

a hydrogen atom in the 3 position of the cyclopentyl group and in the 4 position of a 6- or 7-membered cycloalkyl group may be replaced in each case by a hydroxy, hydroxy-C₁₋₃-alkyl, C₁₋₅-alkoxy, C₁₋₅-alkoxy-C₁₋₃-alkyl, phenyl-C₁₋₃-alkoxy-C₁₋₃-alkyl, amino, C₁₋₅-alkylamino, di-(C₁₋₅-alkyl)amino, phenyl-C₁₋₃-alkylamino, C₁₋₅-alkyl-carbonylamino, benzoylamino, amino-C₁₋₃-alkyl, C₁₋₃-alkylamino-C₁₋₃-alkyl, di-(C₁₋₃-alkyl)amino-C₁₋₃-alkyl, phenyl-C₁₋₃-alkylamino-C₁₋₃-alkyl, C₁₋₃-alkyl-carbonylamino-C₁₋₃-alkyl, benzoylamino-C₁₋₃-alkyl, phenylaminocarbonyl, phenyl-C₁₋₃-alkylaminocarbonyl, carboxy, or C₁₋₃-alkoxy-carbonyl group, or

in each case the methylene group in the 4 position of a 6- or 7-membered cycloalkyl group may be replaced by an oxygen or sulfur atom or by an imino group optionally substituted by a C₁₋₆-alkyl, phenyl, C₁₋₆-alkyl-carbonyl, benzoyl, phenyl-(C₁₋₃-alkyl)-carbonyl, C₁₋₆-alkylaminocarbonyl, di-(C₁ 1₅-alkyl)-aminocarbonyl, phenylaminocarbonyl, N-(C₁₋₃-alkyl)-phenylaminocarbonyl, phenyl-C₁₋₃-alkylaminocarbonyl, or N-(C₁₋₃-alkyl)-phenyl-C₁₋₃-alkylaminocarbonyl group, or

in a 5- or 6-membered cycloalkyl group one or two single bonds separated from each other by at least one bond and separated from position 1 may in each case be fused to a phenyl group, while in a bi-or tricyclic ring system thus formed the hydrogen atom bound to the saturated carbon atom in position 1 may be replaced by a C₁₋₅-alkylaminocarbonyl, di-(C₁₋₅-alkyl)aminocarbonyl, phenyl-C₁₋₃-alkylaminocarbonyl, or C₁₋₅-alkoxy-carbonyl group, wherein terminal methyl groups in each case may be wholly or partially fluorinated,

a C₁₋₆-alkyl group optionally substituted by a C₃₋₇-cycloalkyl group, which is substituted

by a carboxy or C₁₋₃-alkoxy-carbonyl group,

by a phenyl, 1-naphthyl, or 2-naphthyl group,

by a 5-membered heteroaryl group bound via a carbon or nitrogen atom, which contains

an imino group optionally substituted by a C₁₋₃-alkyl, trifluoromethyl, phenyl, phenyl-C₁₋₃-alkyl, C₁₋₃-alkyl-carbonyl, phenylcarbonyl, or phenyl-C₁₋₃-alkyl-carbonyl group, or an oxygen or sulfur atom,

an imino group optionally substituted by a C₁₋₃-alkyl group or an oxygen or sulfur atom,

and additionally a nitrogen atom, or an imino group optionally substituted by a C₁₋₃-alkyl group and two nitrogen atoms, or

an oxygen or sulfur atom and two nitrogen atoms,

by a 6-membered heteroaryl group, which contains one or two nitrogen atoms,

while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety,

while the abovementioned phenyl and naphthyl groups as well as the mono- and bicyclic heteroaryl groups in the carbon skeleton may be monosubstituted by a fluorine, chlorine, or bromine atom, by a C₁₋₅-alkyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, amino-C₁₋₃-alkyl, C₁₋₃-alkylamino-C₁₋₃-alkyl, di-(Ct 1₃-alkyl)amino-C₁₋₃-alkyl, C₁₋₅-alkoxy-carbonylamino-C₁₋₃-alkyl, acetylamino, propionylamino, N-(C₁₋₃-alkyl)benzoylamino, acetyl, propionyl, carboxy, C₁₋₃-alkoxy-carbonyl, C₁₋₃-alkoxy-carbonyl-C₁₋₃-alkyl, aminocarbonyl, C₁₋₃-alkylaminocarbonyl, di-(C₁₋₃-alkyl)aminocarbonyl, or cyano group or, with the exception of 5-membered heteroaryl groups containing more than two heteroatoms, may also be disubstituted by the abovementioned substituents, while the substituents may be identical or different,

a C₁₋₆-alkyl group substituted by a phenyl group and a carboxy, C₁₋₃-alkoxy-carbonyl, aminocarbonyl, C₁₋₃-alkylaminocarbonyl, or di-(C₁₋₃-alkyl)-aminocarbonyl group,

a phenyl-C₂₋₅-alkenylene-CH₂, phenyl-C₂₋₅-alkynylene-CH₂, heteroaryl-C₂₋₅-alkenylene-CH₂, or heteroaryl-C₂₋₅-alkynylene-CH₂ group, wherein a hydrogen atom of the methylene group in position 1 may be replaced by a C₁₋₃-alkyl group and independently thereof the phenyl moiety as well as the heteroaryl moiety may be mono- or disubstituted by fluorine, chlorine, or bromine atoms, by C₁₋₆-alkyl, C₃₋₇-cycloalkyl, trifluoromethyl, C₁₋₃-alkoxy, phenyl, heteroaryl, or cyano groups, while the substituents may be identical or different and disubstitution by two aromatic groups is excluded,

while heteroaryl is a 5-membered heteroaryl group bound via a carbon or nitrogen atom, which contains

an imino group substituted optionally by a C₁₋₃-alkyl group, or an oxygen or sulfur atom,

an imino group substituted optionally by a C₁₋₃-alkyl group, or an oxygen or sulfur atom and additionally a nitrogen atom, or

an imino group substituted optionally by a C₁₋₃-alkyl group and two nitrogen atoms, or

an oxygen or sulfur atom and two nitrogen atoms,

or a 6-membered heteroaryl group, which contains one or two nitrogen atoms,

while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety,

the group R^{b}-A^{b}-E^{b}-C₁₋₃-alkyl optionally substituted in the C₁₋₃-alkyl moiety by a C₁₋₄-alkyl or C₃₋₅-cycloalkyl group, wherein

R^{b} denotes a phenyl group optionally mono- or disubstituted by fluorine, chlorine, bromine, or iodine atoms, by C₁₋₄-alkyl, C₂₋₄-alkenyl, C₂₋₄-alkynyl, C₃₋₇-cycloalkyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, amino-C₁₋₃-alkyl, C₁₋₃-alkylamino-C₁₋₃-alkyl, di-(C₁₋₃-alkyl)amino-C₁₋₃-alkyl, acetylamino, propionylamino, acetyl, propionyl, carboxy, C₁₋₃-alkoxy-carbonyl, C₁₋₃-alkoxy-carbonyl-C₁₋₃-alkyl, aminocarbonyl, C₁₋₃-alkylaminocarbonyl, di-(C₁₋₃-alkyl)aminocarbonyl, or cyano groups, while the substituents may be identical or different,

a 5-membered heteroaryl group which

may be bound via a carbon atom or, if A^{b} denotes a bond, a —CH₂, —(CH₂)₂, sulfonyl, or carbonyl group, may also be bound via a nitrogen atom and which contains

an imino group optionally substituted by a C₁₋₃-alkyl group, or an oxygen or sulfur atom,

an imino group optionally substituted by a C₁₋₃-alkyl group or an oxygen or sulfur atom and additionally a nitrogen atom, or

an imino group optionally substituted by a C₁₋₃-alkyl group and two nitrogen atoms, or

an oxygen or sulfur atom and two nitrogen atoms,

a 6-membered heteroaryl group, which contains one or two nitrogen atoms,

while a phenyl ring may be fused to the abovementioned 5- or 6-membered heteroaryl groups via two adjacent carbon atoms and the bicyclic heteroaryl groups thus formed may be bound via the heteroaromatic or carbocyclic moiety,

while the abovementioned mono- and bicyclic heteroaryl groups may be monosubstituted in the carbon skeleton by a fluorine, chlorine, or bromine atom, by a C₁₋₄-alkyl, C₂₋₄-alkenyl, C₂₋₄-alkynyl, C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)-amino, acetylamino, propionylamino, acetyl, propionyl, C₁₋₃-alkoxy-carbonyl, aminocarbonyl, C₁₋₃-alkylaminocarbonyl, di-(C₁₋₃-alkyl)aminocarbonyl, or cyano group or, with the exception of 5-membered heteroaryl groups containing more than two heteroatoms, may also be disubstituted by the abovementioned substituents, while the substituents may be identical or different,

a C₃₋₇-cycloalkyl group wherein

one or two hydrogen atoms in each case may be replaced by a C₁₋₃-alkyl group and/or

in each case the methylene group in the 4 position of a 6- or 7-membered cycloalkyl group may be replaced by an oxygen or sulfur atom, by a sulfinyl, sulfonyl, or by an imino group optionally substituted by a C₁₋₃-alkyl, C₁₋₃-alkyl-carbonyl, C₁₋₃-alkoxy-carbonyl, C₁₋₃-alkylaminocarbonyl, or di-(C₁₋₃-alkyl)-aminocarbonyl group, or

the two hydrogen atoms of the methylene group in the 3 position of a cyclopentyl group or in 3 or 4 position of a cyclohexyl or cycloheptyl group may be replaced by an n-butylene, n-pentylene, n-hexylene, 1,2-ethylenedioxy, or 1,3-propylenedioxy group and in the rings thus formed one or two hydrogen atoms may be replaced by C₁₋₃-alkyl groups,

a 4- to 7-membered cycloalkyleneimino group wherein

the cycloalkylene moiety may be fused to a phenyl ring, or

one or two hydrogen atoms in each case may be replaced by a C₁₋₃-alkyl group and/or

in each case the carbon atom in the 4 position of a 6- or 7-membered cycloalkyleneimino group may be substituted by a hydroxy-C₁₋₃-alkyl, C₁₋₆-alkoxy-C₁₋₃-alkyl, hydroxycarbonyl, C₁₋₆-alkoxy-carbonyl, aminocarbonyl, C₁₋₃-alkylaminocarbonyl, di-(C₁₋₃-alkyl)-aminocarbonyl-,4- to 7-membered cycloalkyleneimino, phenyl, 4-(C₁₋₃-alkyl)-1,2,4-triazol-3-yl, phenyl-C₁₋₃-alkylamino, or N-(C₁₋₃-alkyl)-phenyl-C₁₋₃-alkylamino group, or

may be replaced by an oxygen or sulfur atom, by a sulfinyl or sulfonyl group, or by an imino group optionally substituted by a C₁₋₃-alkyl, phenyl, C₁₋₃-alkyl-carbonyl, benzoyl, phenyl-C₁₋₃-alkyl-carbonyl, C₁₋₃-alkylaminocarbonyl, di-(C₁₋₃-alkyl)-aminocarbonyl, phenylaminocarbonyl, or N-(C₁₋₃-alkyl)-phenylaminocarbonyl group, or

the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene, n-hexylene, 1,2-ethylenedioxy, or 1,3-propylenedioxy group and in the rings thus formed one or two hydrogen atoms may be replaced by C₁₋₃-alkyl groups, or

in a 5-, 6-, or 7-membered cycloalkyleneimino group a —CH₂— group linked to the imino nitrogen atom may be replaced by a carbonyl group, or a —(CH₂)₂— group linked to the imino nitrogen atom may be replaced by a —CO—NR⁸— group, or a —(CH₂)₃— group linked to the imino nitrogen atom may be replaced by a —CO—NR⁸—CO— group,

while R⁸ denotes a hydrogen atom, or a C₁₋₃-alkyl group,

A^{b} denotes a bond, an oxygen or sulfur atom, an —NH, —N(C₁₋₃-alkyl), sulfinyl, sulfonyl, or a carbonyl group,

one of the groups —CH₂—, —(CH₂)₂—, —O—CH₂—, —CH₂—O—, NH—CH₂—, —CH₂—NH—, —NH—CO—, —CO—NH—, —NH—SO—₂, —SO₂—NH—, —CH═CH—, or —C≡C—

wherein a hydrogen atom bound to a carbon atom and/or a hydrogen atom bound to a nitrogen atom may be replaced by a C₁₋₃-alkyl group in each case and a heteroatom of the group A^{b} is not linked to a nitrogen atom of a 5-membered heteroaryl group of the group R^{b},

E^{b} denotes a phenylene group optionally substituted by a fluorine, chlorine, or bromine atom, by a C₁₋₄-alkyl group, by a trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, amino-C₁₋₃-alkyl, C₁₋₃-alkylamino-C₁₋₃-alkyl, di-(C₁₋₃-alkyl)amino-C₁₋₃-alkyl, acetylamino, propionylamino, acetyl, propionyl, carboxy, C₁₋₃-alkoxy-carbonyl, C₁₋₃-alkoxy-carbonyl-C₁₋₃-alkyl, aminocarbonyl, C₁₋₃-alkylaminocarbonyl, di-(C₁₋₃-alkyl)aminocarbonyl, or cyano group,

the group R^{c}-A^{c}-E^{c}-C₁₋₃-alkyl optionally substituted in the C₁₋₃-alkyl moiety by a C₁₋₄-alkyl or C₃₋₅-cycloalkyl group wherein

R^{c} assumes the meanings given for R^{b} hereinbefore, while any reference to A^{b} must be replaced by a reference to A^{c},

A^{c} assumes the meanings given for A^{b} hereinbefore, while any reference to R^{b} must be replaced by a reference to R^{c},

E^{c} denotes a 5-membered heteroarylene group bound via two carbon atoms or via a carbon atom and an imino-nitrogen atom, while the imino-nitrogen atom of the heteroarylene group is not linked to a heteroatom of the group A^{c} and the heteroarylene group contains

an imino group optionally substituted by a C₁₋₃-alkyl group, an oxygen or sulfur atom,

an imino group optionally substituted by a C₁₋₃-alkyl group or an oxygen or sulfur atom and additionally a nitrogen atom, or

an imino group optionally substituted by a C₁₋₃-alkyl group and two nitrogen atoms, or

an oxygen or sulfur atom and two nitrogen atoms,

or a 6-membered heteroarylene group, which contains one or two nitrogen atoms,

while a phenyl ring may be fused to the abovementioned 5-membered heteroarylene groups containing one or two heteroatoms as well as to the abovementioned 6-membered heteroarylene groups via two adjacent carbon atoms and the bicyclic heteroarylene groups thus formed may be bound via the heteroaromatic and/or carbocyclic moiety,

and while the abovementioned mono- and bicyclic heteroarylene groups in the carbon skeleton may be substituted by a fluorine, chlorine, or bromine atom, by a C₁₋₄-alkyl group, by a C₃₋₇-cycloalkyl, trifluoromethyl, hydroxy, C₁₋₃-alkoxy, trifluoromethoxy, amino, C₁₋₃-alkylamino, acetylamino, propionylamino, acetyl, propionyl, C₁₋₃-alkoxy-carbonyl, aminocarbonyl, C₁₋₃-alkylaminocarbonyl, or cyano group,

or R⁶ and R⁷ together denote an n-alkylene bridge with 3 to 6 carbon atoms, wherein

one or two hydrogen atoms in each case may be replaced by a C₁₋₃-alkyl group and/or

a —CH₂—CH₂— group may be replaced by a 1,2-linked phenylene group which may be mono- or disubstituted by fluorine, chlorine, or bromine atoms, by C₁₋₃-alkyl, trifluoromethyl, hydroxy, C₁₋₃-alkoxy, trifluoromethoxy, amino, C₁₋₃-alkylamino, di-(C₁₋₃-alkyl)amino, acetylamino, propionylamino, acetyl, propionyl, C₁₋₃-alkoxy-carbonyl, aminocarbonyl, C₁₋₃-alkylaminocarbonyl, cyano, phenyloxy, or phenyl-C₁₋₃-alkyl groups, while disubstitution with the last-named group is excluded,

while the abovementioned phenyloxy- and phenyl-C₁₋₃-alkyl group in the phenyl moiety may in turn be substituted by a fluorine, chlorine, or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino, or cyano group,

or in each case the carbon atom in the 3 position of an n-pentylene or n-hexylene group may be monosubstituted by a C₁₋₃-alkyl group terminally substituted by a phenyl, cyano, hydroxy, C₁₋₃-alkoxy, amino, C₁₋₃-alkylamino, di-(C₁₋₃-alkyl)-amino, or a 5- to 7-membered cycloalkyleneimino group, by a carboxy, C₁₋₃-alkoxy-carbonyl, amino-C₁₋₃-alkyl, C₁₋₃-alkylamino-C₁₋₃-alkyl, N—C₁₋₃-alkyl-N-(C₁₋₃-alkyl-carbonyl)-amino-C₁₋₃-alkyl, di-(C₁₋₃-alkyl)-amino-C₁₋₃-alkyl, aminocarbonyl, C₁₋₃-alkylaminocarbonyl, or di-(C₁₋₃-alkyl)-aminocarbonyl group, or may be disubstituted by a phenyl group and a cyano, hydroxy, or C₁₋₃-alkoxy group, or

the methylene group in the 3 position of an n-pentylene or n-hexylene group may be replaced by an oxygen or sulfur atom, by a sulfinyl or sulfonyl group, or by an imino group optionally substituted by a C₁₋₃-alkyl, phenyl-C₁₋₃-alkyl, C₁₋₃-alkyl-carbonyl, benzoyl, C₁₋₃-alkylaminocarbonyl, di-(C₁₋₃-alkyl)-aminocarbonyl, phenylaminocarbonyl, or N-(C₁₋₃-alkyl)-phenylaminocarbonyl group, or

a methylene group in position 1 of an n-butylene, n-pentylene, or n-hexylene group may be replaced by a carbonyl group,

while the phenyl groups mentioned as being unsubstituted or monosubstituted in the definition of the abovementioned groups as well as aromatic or heteroaromatic parts of molecules may, unless otherwise stated, optionally additionally be substituted in the carbon skeleton by fluorine, chlorine, or bromine atoms, by C₁₋₃-alkyl groups, by trifluoromethyl, hydroxy, C₁₋₃-alkoxy, trifluoromethoxy, amino, C₁₋₃-alkylamino, acetylamino, acetyl, C₁₋₃-alkoxy-carbonyl, aminocarbonyl, C₁₋₃-alkylaminocarbonyl, or cyano groups, while the substituents may be identical or different and the resulting aromatic groups and parts of molecules may be at most disubstituted,

the hydrogen atoms in the C₁₋₃-alkyl and alkoxy groups mentioned in the definition of the above groups may be wholly or partially replaced by fluorine atoms, and

the alkyl and alkoxy groups mentioned in the definition of the above groups or in the alkyl moieties contained in the groups of formula 1 defined above with more than two carbon atoms may be straight-chain or branched, unless otherwise specified.

The carboxy groups mentioned in the definition of the abovementioned groups may be replaced by a group which can be converted into a carboxy group in vivo or by a group which is negatively charged under physiological conditions,

and furthermore the amino and imino groups mentioned in the definition of the above-mentioned groups may be substituted by a group which can be cleaved in vivo. Such groups are described for example in WO 98/46576 and by N. M. Nielsen et al. in International Journal of Pharmaceutics 39, 75-85 (1987).

By a group which can be converted in vivo into a carboxy group is meant, for example, a hydroxymethyl group, a carboxy group esterified with an alcohol wherein the alcohol moiety is preferably a C₁₋₆-alkanol, a phenyl-C₁₋₃-alkanol, a C₃₋₉-cycloalkanol, while a C₅₋₈-cycloalkanol may additionally be substituted by one or two C₁₋₃-alkyl groups, a C₅₋₈-cycloalkanol wherein a methylene group in the 3 or 4 position is replaced by an oxygen atom or by an imino group optionally substituted by a C₁₋₃-alkyl, phenyl-C₁₋₃-alkyl, phenyl-C₁₋₃-alkoxy-carbonyl, or C₂₋₆-alkanoyl group and the cycloalkanol moiety may additionally be substituted by one or two C₁₋₃-alkyl groups, a C₄₋₇-cycloalkenol, a C₃₋₅-alkenol, a phenyl-C₃₋₅-alkenol, a C₃₋₅-alkynol, or phenyl-C₃₋₅-alkynol with the proviso that no bonds to the oxygen atom start from a carbon atom which carries a double or triple bond, a C₃₋₈-cycloalkyl-C₁₋₃-alkanol, a bicycloalkanol with a total of 8 to 10 carbon atoms which may additionally be substituted in the bicycloalkyl moiety by one or two C₁₋₃-alkyl groups, a 1,3-dihydro-3-oxo-1-isobenzofuranol, or an alcohol of formula

Rₚ—CO—O—(R_{q}CRᵣ)—OH,

wherein:

Rₚ denotes a C₁₋₈-alkyl, C₅₋₇-cycloalkyl, phenyl, or phenyl-C₁₋₃-alkyl group,

R_{q} denotes a hydrogen atom, a C₁₋₃-alkyl, C₅₋₇-cycloalkyl, or phenyl group, and

Rᵣ denotes a hydrogen atom or a C₁₋₃-alkyl group,

by a group which is negatively charged under physiological conditions is meant, for example, a tetrazol-5-yl, phenylcarbonylaminocarbonyl, trifluoromethylcarbonylaminocarbonyl, C₁₋₆-alkylsulfonylamino, phenylsulfonylamino, benzylsulfonylamino, trifluoromethyl-sulfonylamino, C₁₋₆-alkylsulfonylaminocarbonyl, phenylsulfonylaminocarbonyl, benzylsulfonylaminocarbonyl, or perfluoro-C₁₋₆-alkylsulfonylaminocarbonyl group

and by a group which can be cleaved in vivo from an imino or amino group is meant, for example, a hydroxy group, an acyl group such as a phenylcarbonyl group optionally mono- or disubstituted by fluorine, chlorine, bromine, or iodine atoms, by C₁₋₃-alkyl or C₁₋₃-alkoxy groups, while the substituents may be identical or different, a pyridinoyl group or a C₁₋₁₆-alkanoyl group such as the formyl, acetyl, propionyl, butanoyl, pentanoyl, or hexanoyl group, a 3,3,3-trichloropropionyl or allyloxycarbonyl group, a C₁₋₆-alkoxy-carbonyl or C₁₋₁₆-alkyl-carbonyloxy group, wherein hydrogen atoms may be wholly or partially replaced by fluorine or chlorine atoms such as the methoxycarbonyl, ethoxycarbonyl, propoxycarbonyl, isopropoxycarbonyl, butoxycarbonyl, tert-butoxycarbonyl, pentoxycarbonyl, hexoxycarbonyl, octyloxycarbonyl, nonyloxycarbonyl, decyloxycarbonyl, undecyloxycarbonyl, dodecyloxycarbonyl, hexadecyloxycarbonyl, methylcarbonyloxy, ethylcarbonyloxy, 2,2,2-trichloroethylcarbonyloxy, propylcarbonyloxy, isopropylcarbonyloxy, butylcarbonyloxy, tert-butylcarbonyloxy, pentylcarbonyloxy, hexylcarbonyloxy, octylcarbonyloxy, nonylcarbonyloxy, decylcarbonyloxy, undecylcarbonyloxy, dodecylcarbonyloxy, or hexadecylcarbonyloxy group, a phenyl-C₁₋₆-alkoxy-carbonyl group such as the benzyloxycarbonyl, phenylethoxycarbonyl, or phenylpropoxycarbonyl group, a 3-aminopropionyl group wherein the amino group may be mono- or disubstituted by C₁₋₆-alkyl or C₃₋₇-cycloalkyl groups and the substituents may be identical or different, a C₁₋₃-alkylsulfonyl-C₂₋₄-alkoxy-carbonyl, C₁₋₃-alkoxy-C₂₋₄-alkoxy-C₂₋₄-alkoxy-carbonyl, Rₚ—CO—O—(R^{c}R^{r})—O—CO, C₁₋₆-alkyl-CO—NH—(RₛCRₜ)—O—CO—, or C₁₋₆-alkyl-CO—O—(RₛCRₜ)—(RₛCRₜ)—O—CO— group, wherein Rₚ to Rᵣ are as hereinbefore defined,

Rₛ and Rₜ, which may be identical or different, denote hydrogen atoms or C₁₋₃-alkyl groups.

Preferred compounds of the above general formula I are those wherein

X₁ to X₄ are as hereinbefore defined,

A^{a} denotes a bond, an oxygen atom, a —NH, —N(C₁₋₃-alkyl), sulfonyl, or carbonyl group,

one of the groups —CH₂—, —(CH₂)₂—, —NH—CH₂—, —CH₂—NH—, —NH—CO—, —CO—NH—, —NH—SO₂—, or —SO₂—NH—,

wherein a hydrogen atom bound to a carbon atom and/or a hydrogen atom bound to a nitrogen atom may be replaced in each case by a C₁₋₃-alkyl group and a heteroatom of group A^{a} is not linked to a nitrogen atom of a 5-membered heteroaryl group of the group R^{a},

R^{a} denotes a phenyl group,

a 5-membered heteroaryl group bound via a carbon or nitrogen atom which contains

an imino group optionally substituted by a C₁₋₄-alkyl or C₁₋₄-alkyl-carbonyl group, an oxygen or sulfur atom, or

an imino group optionally substituted by a C₁₋₄-alkyl group or an oxygen or sulfur atom and additionally a nitrogen atom,

a 6-membered heteroaryl group, which contains one or two nitrogen atoms,

while the abovementioned phenyl and heteroaryl groups may be substituted in the carbon skeleton by a fluorine, chlorine, or bromine atom, by a C₁₋₄-alkyl group, by a C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)amino, acetylamino, N-(C₁₋₃-alkyl)-acetylamino, acetyl, or cyano group,

a C₃₋₇-cycloalkyl group, wherein

the methylene group in the 4 position of a 6-membered cycloalkyl group may be replaced by an oxygen or sulfur atom, by a sulfonyl group, or by an imino group optionally substituted by a C₁₋₃-alkyl, phenyl, C₁₋₄-alkyl-carbonyl, or C₁₋₄-alkoxy-carbonyl group,

a 4- to 7-membered cycloalkyleneimino group wherein

one or two hydrogen atoms in each case may be replaced by a C₁₋₃-alkyl group and/or

in each case the methylene group in the 4 position of a 6- or 7-membered cycloalkyleneimino group may be replaced by an oxygen or sulfur atom, by a sulfonyl group, or by an imino group optionally substituted by a C₁₋₅-alkyl, phenyl, C₁₋₄-alkyl-carbonyl, C₁₋₄-alkoxy-carbonyl, C₁₋₃-alkylaminocarbonyl, or di-(C₁₋₃-alkyl)-aminocarbonyl group, or

in a 5-, 6-, or 7-membered cycloalkyleneimino group a —CH₂— group linked to the imino nitrogen atom may be replaced by a carbonyl group, or a —(CH₂)₂— group linked to the imino nitrogen atom may be replaced by a —CO—NR⁸— group, or a —(CH₂)₃— group linked to the imino nitrogen atom may be replaced by a —CO—NR⁸—CO— group,

while R⁸ denotes a hydrogen atom or a C₁₋₃-alkyl group,

R⁵ denotes a hydrogen atom or a C₁₋₃-alkyl group,

Het denotes a 5-membered heteroarylene group bound via two carbon atoms which contains

an imino group substituted by the group R⁹, an oxygen or sulfur atom, or

an imino group substituted by the group R⁹ or an oxygen or sulfur atom and additionally a nitrogen atom,

while R⁹ denotes a hydrogen atom, a C₁₋₅-alkyl group, a —C₂₋₃-alkyl group terminally substituted by an amino, C₁₋₃-alkylamino, di-(C₁₋₃-alkyl)-amino, or C₁₋₅-alkoxy-carbonylamino group, a carboxy-C₁₋₃-alkyl, C₁₋₃-alkoxy-carbonyl-C₁₋₃-alkyl, phenyl, phenyl-C₁₋₃-alkyl, C₁₋₅-alkyl-carbonyl, or phenylcarbonyl group, or R⁹ together with R⁶ denotes a —(CH₂)ₚ— bridge wherein p denotes the number 2 or 3,

or an imino group optionally substituted by a C₁₋₃-alkyl group and two nitrogen atoms, or

an oxygen or sulfur atom and two nitrogen atoms,

or a 6-membered heteroarylene group, which contains one or two nitrogen atoms,

while the abovementioned heteroarylene groups in the carbon skeleton may be substituted by a fluorine, chlorine, or bromine atom, by a C₁₋₃-alkyl group, by a cyclopropyl, trifluoromethyl, C₁₋₃-alkoxy, trifluoromethoxy, C₁₋₃-alkylamino, di-(C₁₋₃-alkyl)amino, acetylamino, N-(C₁₋₃-alkyl)-acetylamino, acetyl, C₁₋₃-alkylaminocarbonyl, or di-(C₁₋₃-alkyl)aminocarbonyl group,

R⁶ denotes a hydrogen atom or a C₁₋₄-alkyl group,

R⁷ denotes a C₁₋₆-alkyl group,

a straight-chain C₂₋₆-alkyl group which is terminally substituted by an amino, C₁₋₃-alkylamino or di-(C₁₋₃-alkyl)-amino group,

a C₁₋₆-alkyl group substituted by an C₃₋₇-cycloalkyl group, while

a hydrogen atom in the 3 position of the cyclopentyl group and in the 4 position of a 6- or 7-membered cycloalkyl group may be replaced in each case by a C₁₋₅-alkoxy, phenyl-C₁₋₃-alkoxy-C₁₋₃-alkyl, phenyl-C₁₋₃-alkylamino, C₁₋₅-alkyl-carbonylamino, benzoylamino, phenyl-C₁₋₃-alkylamino-C₁₋₃-alkyl, benzoylamino-C₁₋₃-alkyl, phenylaminocarbonyl, phenyl-C₁₋₃-alkylaminocarbonyl, carboxy, or C₁₋₃-alkoxy-carbonyl group, or

in each case the methylene group in the 4 position of a 6- or 7-membered cycloalkyl group may be replaced by an imino group optionally substituted by a phenyl, C₁₋₆-alkyl-carbonyl, benzoyl, phenyl-(C₁₋₃-alkyl)-carbonyl, phenylaminocarbonyl, N-(C₁₋₃-alkyl)-phenylaminocarbonyl, phenyl-C₁₋₃-alkylaminocarbonyl, or N-(C₁₋₃-alkyl)-phenyl-C₁₋₃-alkylaminocarbonyl group, or

in a 5- or 6-membered cycloalkyl group one or two single bonds separated by at least one bond from each other and from position 1 may each be fused to a phenyl group, while in a bi- or tricyclic ring system thus formed the hydrogen atom bound to the saturated carbon atom in position 1 may be replaced by a C₁₋₃-alkylaminocarbonyl, di-(C₁₋₃-alkyl)aminocarbonyl, or

C₁₋₅-alkoxy-carbonyl group, while terminal methyl groups in each case may be wholly or partly fluorinated,

a C₁₋₆-alkyl group optionally substituted by a C₃₋₇-cycloalkyl group which is substituted

by a carboxy or C₁₋₃-alkoxy-carbonyl group,

by a phenyl, 1-naphthyl, or 2-naphthyl group,

by a 5-membered heteroaryl group bound via a carbon or nitrogen atom which contains

an imino group optionally substituted by a C₁₋₃-alkyl or trifluoromethyl group, an oxygen or sulfur atom, or

an imino group optionally substituted by a C₁₋₃-alkyl group or an oxygen or sulfur atom and additionally a nitrogen atom,

by a 6-membered heteroaryl group, which contains one or two nitrogen atoms,

while the abovementioned phenyl groups as well as the heteroaryl groups in the carbon skeleton may be monosubstituted by a fluorine, chlorine, or bromine atom, by a C₁₋₃-alkyl, trifluoromethyl, C₁₋₃-alkoxy, fluoromethoxy, difluoromethoxy, trifluoromethoxy, C₁₋₃-alkylamino, di-(C₁₋₃-alkyl)amino, amino-C₁₋₃-alkyl, acetylamino, acetyl, C₁₋₃-alkoxy-carbonyl-C₁₋₃-alkyl, C₁₋₅-alkoxy-carbonylamino-C₁₋₃-alkyl, C₁₋₃-alkylaminocarbonyl, or di-(C₁₋₃-alkyl)aminocarbonyl group, or may also be disubstituted by the abovementioned substituents, while the substituents may be identical or different,

a C₁₋₆-alkyl group substituted by a phenyl group and a carboxy, C₁₋₃-alkoxy-carbonyl, aminocarbonyl, C₁₋₃-alkylaminocarbonyl, or di-(C₁₋₃-alkyl)-aminocarbonyl group,

a phenyl-C₂₋₃-alkenylene-CH₂, or phenyl-C₂₋₃-alkynylene-CH₂ group, wherein a hydrogen atom of the methylene group in the 1 position may be replaced by a methyl group and independently thereof the phenyl moiety may be substituted by a fluorine, chlorine, or bromine atom, by a C₁₋₄-alkyl, C₃₋₇-cycloalkyl, trifluoromethyl, C₁₋₃-alkoxy, phenyl, pyridyl, pyrimidinyl, pyrazinyl, thienyl, pyrrolyl, pyrazolyl, or thiazolyl group,

the group R^{b}-A^{b}-E^{b}-C₁₋₃-alkyl optionally substituted by a methyl group in the C₁₋₃-alkyl moiety, wherein

R^{b} denotes a phenyl group optionally mono- or disubstituted by fluorine, chlorine, or bromine atoms, by C₁₋₃-alkyl, cyclopropyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, acetylamino, acetyl, carboxy, C₁₋₃-alkoxy-carbonyl, aminocarbonyl, C₁₋₃-alkylaminocarbonyl, di-(C₁₋₃-alkyl)aminocarbonyl, or cyano groups, while the substituents may be identical or different,

a 5-membered heteroaryl group which

may be bound via a carbon atom or, if A^{b} denotes a bond, a —CH₂, —(CH₂)₂, sulfonyl, or carbonyl group, may also be bound via a nitrogen atom, and

contains an imino group optionally substituted by a C₁₋₃-alkyl group, an oxygen or sulfur atom,

an imino group optionally substituted by a C₁₋₃-alkyl group or an oxygen or sulfur atom and additionally a nitrogen atom, or

an imino group optionally substituted by a C₁₋₃-alkyl group and two nitrogen atoms, or

an oxygen or sulfur atom and two nitrogen atoms,

a 6-membered heteroaryl group, which contains one or two nitrogen atoms,

while the abovementioned heteroaryl groups in the carbon skeleton may be monosubstituted by a fluorine, chlorine, or bromine atom, by a C₁₋₄-alkyl, C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)-amino, acetylamino, acetyl, C₁₋₃-alkoxy-carbonyl, aminocarbonyl, C₁₋₃-alkylaminocarbonyl, or di-(C₁₋₃-alkyl)aminocarbonyl group or, with the exception of 5-membered heteroaryl groups containing more than two heteroatoms, may also be disubstituted by the abovementioned substituents, while the substituents may be identical or different,

a C₃₋₇-Cycloalkyl group wherein

one or two hydrogen atoms in each case may be replaced by a C₁₋₃-alkyl group and/or

the methylene group in the 4 position of a cyclohexyl group may be replaced by an oxygen atom, by a sulfonyl group, or by an imino group optionally substituted by a C₁₋₃-alkyl, C₁₋₃-alkyl-carbonyl, C₁₋₃-alkoxy-carbonyl, C₁₋₃-alkylaminocarbonyl, or di-(C₁₋₃-alkyl)-aminocarbonyl group, or

the two hydrogen atoms of the methylene group in the 3 position of a cyclopentyl group, or in the 3 or 4 position of a cyclohexyl or cycloheptyl group may be replaced by an n-butylene, n-pentylene, n-hexylene, 1,2-ethylenedioxy, or 1,3-propylenedioxy group,

a 4- to 7-membered cycloalkyleneimino group wherein

the cycloalkylene moiety may be fused to a phenyl ring, or

one or two hydrogen atoms in each case may be replaced by a C₁₋₃-alkyl group and/or

in each case the carbon atom in the 4 position of a 6- or 7-membered cycloalkyleneimino group may be substituted by a 4- to 7-membered cycloalkyleneimino, phenyl, or 4-(C₁₋₃-alkyl)-1,2,4-triazol-3-yl group, or

may be replaced by an oxygen atom, by a sulfonyl group, or by an imino group optionally substituted by a C₁₋₃-alkyl, C₁₋₃-alkyl-carbonyl, C₁₋₃-alkylaminocarbonyl, or di-(C₁₋₃-alkyl)-aminocarbonyl group, or

the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene, n-hexylene, 1,2-ethylenedioxy, or 1,3-propylenedioxy group, or

in a 5-, 6-, or 7-membered cycloalkyleneimino group a —CH₂— group linked to the imino nitrogen atom may be replaced by a carbonyl group

A^{b} denotes a bond, an oxygen atom, a —NH, —N(C₁₋₃-alkyl), sulfonyl, or a carbonyl group,

one of the groups —CH₂—, —(CH₂)₂—, —C≡C—, —O—CH₂—, —CH₂—O—, NH—CH₂—, —CH₂—NH—, —NH—CO—, —CO—NH—, —NH—SO₂—, —SO₂—NH—,

wherein a hydrogen atom bound to a carbon atom and/or a hydrogen atom bound to a nitrogen atom may be replaced by a C₁₋₃-alkyl group in each case and a heteroatom of group A^{b} is not linked to a nitrogen atom of a 5-membered heteroaryl group of the group R^{b}, and

E^{b} denotes a phenylene group optionally substituted by a fluorine, chlorine, or bromine atom, by a C₁₋₄-alkyl group, by a trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, acetylamino, acetyl, carboxy, C₁₋₃-alkoxy-carbonyl, C₁₋₃-alkoxy-carbonyl-C₁₋₃-alkyl, aminocarbonyl, C₁₋₃-alkylaminocarbonyl, di-(C₁₋₃-alkyl)aminocarbonyl, or cyano group, or

the group R^{c}-A^{c}-E^{c}-C₁₋₃-alkyl, wherein

R^{c} has the meanings given for R^{b} hereinbefore, while any reference to A^{b} must be replaced by a reference to A^{c},

A^{c} denotes a bond, an oxygen atom, a —CH₂, —NH, —N(C₁₋₃-alkyl), —NH—CO, —CO—NH, or carbonyl group,

while a heteroatom of the group A^{c} is not linked to a nitrogen atom of a 5-membered heteroaryl group of the group R^{c}, and

E^{c} denotes a 5-membered heteroarylene group bound via two carbon atoms or via a carbon atom and an imino-nitrogen atom, while the imino-nitrogen atom of the heteroarylene group is not linked to a heteroatom of the group A^{c} and the heteroarylene group contains

an imino group optionally substituted by a C₁₋₃-alkyl group, an oxygen or sulfur atom,

an imino group optionally substituted by a C₁₋₃-alkyl group, or an oxygen or sulfur atom and additionally a nitrogen atom, or

an imino group optionally substituted by a C₁₋₃-alkyl group and two nitrogen atoms, or

an oxygen or sulfur atom and two nitrogen atoms,

or a 6-membered heteroarylene group, which contains one or two nitrogen atoms,

while the abovementioned 5- and 6-membered heteroarylene groups in the carbon skeleton may be substituted by a fluorine, chlorine, or bromine atom, by a C₁₋₄-alkyl group, by a C₃₋₇-cycloalkyl, trifluoromethyl, hydroxy, C₁₋₃-alkoxy, trifluoromethoxy, amino, C₁₋₃-alkylamino, acetylamino, acetyl, C₁₋₃-alkoxy-carbonyl, aminocarbonyl, C₁₋₃-alkylaminocarbonyl, or cyano group,

or R⁶ and R⁷ together denote an n-alkylene bridge with 4 or 5 carbon atoms wherein

a hydrogen atom may be replaced by a C₁₋₃-alkyl group and/or

a —CH₂—CH₂ group may be replaced by a 1,2-linked phenylene group, which may be substituted by a fluorine, chlorine, or bromine atom, by a C₁₋₃-alkyl, trifluoromethyl, hydroxy, C₁₋₃-alkoxy, trifluoromethoxy, amino, C₁₋₃-alkylamino, di-(C₁₋₃-alkyl)amino, acetylamino, acetyl, C₁₋₃-alkoxy-carbonyl, aminocarbonyl, C₁₋₃-alkylaminocarbonyl, or cyano group, or by a phenyloxy or phenyl-C₁₋₃-alkyl group optionally substituted in the phenyl moiety by a fluorine, chlorine, or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino, or cyano group,

or the carbon atom in the 3 position of an n-pentylene group may be monosubstituted by a C₁₋₃-alkyl group terminally substituted by an amino, C₁₋₃-alkylamino, di-(C₁₋₃-alkyl)-amino, or a 5- to 7-membered cycloalkyleneimino group, by a phenyl, C₁₋₃-alkoxy-carbonyl, aminocarbonyl, C₁₋₃-alkylaminocarbonyl, or di-(C₁₋₃-alkyl)-aminocarbonyl group, or may be disubstituted by a phenyl group and a cyano group, or

the methylene group in the 3 position of an n-pentylene group may be replaced by an oxygen atom, by a sulfonyl group, or by an imino group optionally substituted by a C₁₋₃-alkyl or C₁₋₃-alkyl-carbonyl group,

while the phenyl groups mentioned as being unsubstituted or monosubstituted in the definition of the abovementioned groups as well as aromatic or heteroaromatic parts of molecules may, unless otherwise stated, optionally additionally be substituted in the carbon skeleton by a fluorine, chlorine, or bromine atom, by a C₁₋₃-alkyl group, by a trifluoromethyl, hydroxy, C₁₋₃-alkoxy, trifluoromethoxy, amino, C₁₋₃-alkylamino, acetylamino, acetyl, C₁₋₃-alkoxy-carbonyl, aminocarbonyl, C₁₋₃-alkylaminocarbonyl, or cyano group,

the alkyl and alkoxy groups mentioned in the definition of the above groups or in the alkyl moieties contained in the groups of formula I defined above with more than two carbon atoms may be straight-chain or branched, unless otherwise specified,

the carboxy groups mentioned in the definition of the abovementioned groups may be replaced by a group which can be converted into a carboxy group in vivo or by a group which is negatively charged under physiological conditions, and/or

the amino and imino groups mentioned in the definition of the abovementioned groups may be substituted by a group which can be cleaved in vivo,

their tautomers, their diastereomers, their enantiomers, the mixtures and the salts thereof.

Particularly preferred compounds of the above general formula I are those wherein

X₁ denotes the group CR¹,

X₂ denotes the group CR²,

X₃ denotes the group CR³, and

X₄ denotes the group CR⁴, or

one of the groups X₁ to X₄ denotes a nitrogen atom and the remainder of the groups X₁ to X₄ denote three of the groups CR¹ to CR⁴,

while R¹, R², R³ and R⁴ in each case denote a hydrogen atom, or

one or two of the groups R¹ to R⁴ independently of one another in each case denote a fluorine, chlorine, or bromine atom, a C₁₋₃-alkyl group, a trifluoromethyl, amino, C₁₋₃-alkylamino, or di-(C₁₋₃-alkyl)-amino group and the remainder of the groups R¹ to R⁴ in each case represent a hydrogen atom,

while R⁴ additionally together with R⁵ may assume the meaning of a —(CH₂)ₙ— bridge wherein n denotes the number 1, 2, or 3, and

A^{a} denotes a bond, an oxygen atom, a —CH₂—, —(CH₂)₂—, —NH—, —N(C₁₋₃-alkyl), -sulfonyl-, or carbonyl group, or an —NH—CH₂—, —NH—CO—, —NH—SO₂ group linked to the group R^{a} in formula I via the carbon or sulfur atom,

while a heteroatom of the group A^{a} is not linked to a nitrogen atom of a 5-membered heteroaryl group of the group R^{a},

R^{a} denotes a phenyl or pyridinyl group,

a pyrrolyl, furanyl, thienyl, pyrazolyl, imidazolyl, oxazolyl, isoxazolyl, or thiazolyl group bound via a carbon or nitrogen atom,

while a nitrogen atom of the pyrrolyl, pyrazolyl and imidazolyl group may be substituted by a C₁₋₃-alkyl group and the phenyl group as well as the abovementioned heteroaromatic groups in the carbon skeleton may be substituted by a fluorine, chlorine, or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, or cyano group,

a 5- to 7-membered cycloalkyleneimino group wherein

the methylene group in the 4 position of a 6-membered cycloalkyleneimino group may be substituted by a methyl group or replaced by an oxygen or sulfur atom or by an imino group optionally substituted by a C₁₋₃-alkyl group, or

in a piperidino group a —CH₂— group linked to the imino nitrogen atom may be replaced by a carbonyl group, or a —(CH₂)₂— group linked to the imino nitrogen atom may be replaced by a —CO—NR⁸— group, or a —(CH₂)₃— group linked to the imino nitrogen atom may be replaced by a —CO—NR⁸—CO— group,

while R⁸ denotes a hydrogen atom or a C₁₋₃-alkyl group,

R⁵ denotes a hydrogen atom or a C₁₋₃-alkyl group,

Het denotes a 5-membered heteroarylene group bound via two carbon atoms which contains

an imino group substituted by the group R⁹, an oxygen or sulfur atom, or

an imino group substituted by the group R⁹ or an oxygen or sulfur atom and additionally contains a nitrogen atom,

while R⁹ denotes a hydrogen atom, a C₁₋₃-alkyl group, a —C₂₋₃-alkyl group terminally substituted by an amino, C₁₋₃-alkylamino, di-(C₁₋₃-alkyl)-amino, or C₁₋₄-alkoxy-carbonylamino group, a carboxy-C₁₋₃-alkyl, C₁₋₃-alkoxy-carbonyl-C₁₋₃-alkyl, or C₁₋₃-alkyl-carbonyl group, or R⁹ together with R⁶ denotes a —(CH₂)ₚ— bridge wherein p is the number 2 or 3,

or a pyridinylene or pyrimidinylene group,

while the abovementioned heteroarylene groups in the carbon skeleton may be substituted by a fluorine, chlorine, or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino, or cyano group,

R⁶ denotes a hydrogen atom or a C₁₋₃-alkyl group,

R⁷ denotes a C₁₋₆-alkyl group,

a straight-chain C₂₋₆-alkyl group which is terminally substituted by an amino, C₁₋₃-alkylamino or di-(C₁₋₃-alkyl)-amino group,

a C₁₋₄-alkyl group terminally substituted by a C₃₋₇-cycloalkyl group, while

a hydrogen atom in the 4 position of a cyclohexyl group may be replaced by a C₁₋₅-alkoxy, C₁₋₃-alkoxy-C₁₋₃-alkyl, phenyl-C₁₋₃-alkoxy-methyl, phenyl-C₁₋₃-alkylamino, phenyl-C₁₋₂-alkyl-carbonylamino, benzoylamino, phenylaminocarbonyl, phenyl-C₁₋₃-alkylaminocarbonyl, carboxy, or C₁₋₃-alkoxy-carbonyl group, or

in a cyclopentyl group one or two single bonds separated from each other and from position 1 by at least one bond may each be fused to a phenyl group, while in a bi-or tricyclic ring system thus formed the hydrogen atom bound to the saturated carbon atom in the 1 position may be replaced by a C₁₋₃-alkylaminocarbonyl or di-(C₁₋₃-alkyl)aminocarbonyl group, wherein terminal methyl groups in each case may be wholly or partly fluorinated,

a C₁₋₆-alkyl group optionally substituted by a C₃₋₅-cycloalkyl group which is substituted

by a carboxy or C₁₋₃-alkoxy-carbonyl group, or

by a phenyl, 1-naphthyl, 2-naphthyl, pyridinyl, pyrimidinyl, pyrrolyl, furanyl, thienyl, pyrazolyl, imidazolyl, oxazolyl, isoxazolyl, thiazolyl, or isothiazolyl group,

while a nitrogen atom of the pyrrolyl, pyrazolyl and imidazolyl group may be substituted by a C₁₋₃-alkyl or trifluoromethyl group and the phenyl group as well as the abovementioned heteroaromatic groups in the carbon skeleton may be substituted by a fluorine, chlorine, or bromine atom, by a C₁₋₄-alkyl, trifluoromethyl, C₁₋₃-alkoxy, fluoromethoxy, difluoromethoxy, trifluoromethoxy, C₁₋₄-alkoxy-carbonylamino-C₁₋₃-alkyl, amino, C₁₋₃-alkylamino, di-(C₁₋₃-alkyl)amino, or cyano group,

a C₁₋₆-alkyl group substituted by a phenyl group and a carboxy or C₁₋₃-alkoxy-carbonyl group,

a phenyl-C₂₋₃-alkynylene-CH₂ group wherein a hydrogen atom of the methylene group in the 1 position may be replaced by a methyl group and independently thereof the phenyl moiety may be substituted by a fluorine, chlorine, or bromine atom, or by a C₁₋₄-alkyl, trifluoromethyl, C₁₋₃-alkoxy, phenyl, or cyano group,

the group R^{b}-A^{b}-E^{b}-C₁₋₃-alkyl optionally substituted in the C₁₋₃-alkyl moiety by a methyl group, wherein

R^{b} denotes a phenyl group optionally substituted by a fluorine, chlorine, or bromine atom, by a C₁₋₃-alkyl, trifluoromethyl, hydroxy, C₁₋₃-alkoxy, fluoromethoxy, difluoromethoxy, trifluoromethoxy, carboxy, or C₁₋₃-alkoxy-carbonyl group,

a 5-membered heteroaryl group which

may be bound via a carbon atom or, if A^{b} denotes a bond, may also be bound via a nitrogen atom and contains

an imino group optionally substituted by a C₁₋₃-alkyl group, an oxygen or sulfur atom,

an imino group optionally substituted by a C₁₋₃-alkyl group, or an oxygen or sulfur atom and additionally a nitrogen atom, or

an imino group optionally substituted by a C₁₋₃-alkyl group and two nitrogen atoms, or

an oxygen or sulfur atom and two nitrogen atoms,

a 6-membered heteroaryl group, which contains one or two nitrogen atoms,

while the abovementioned heteroaryl groups may be monosubstituted in the carbon skeleton by a fluorine, chlorine, or bromine atom, by a C₁₋₃-alkyl, trifluoromethyl, phenyl, C₁₋₃-alkoxy, trifluoromethoxy, amino, C₁₋₃-alkylamino, di-(C₁₋₃-alkyl)-amino, or acetylamino group or, with the exception of 5-membered heteroaryl groups containing more than two heteroatoms, may also be disubstituted by a C₁₋₄-alkyl group and one substituent selected from fluorine, chlorine, bromine, C₁₋₃-alkyl, trifluoromethyl, phenyl, C₁₋₃-alkoxy and trifluoromethoxy,

a C₃₋₆-cycloalkyl group, wherein

the two hydrogen atoms of the methylene group in the 3 position of a cyclopentyl group or in the 3 or 4 position of a cyclohexyl group may be replaced by an n-butylene, n-pentylene, or 1,2-ethylenedioxy group,

a 5- to 7-membered cycloalkyleneimino group wherein

the cycloalkylene moiety may be fused to a phenyl ring, or

a hydrogen atom may be replaced by a C₁₋₃-alkyl group and/or

in each case the carbon atom in the 4 position of a 6- or 7-membered cycloalkyleneimino group may be substituted by a 4- to 7-membered cycloalkyleneimino, phenyl, or 4-(C₁₋₃-alkyl)-1,2,4-triazol-3-yl group, or

the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene, or 1,2-ethylenedioxy group,

A^{b} denotes a bond, an oxygen atom, a —CH₂—, —NH—, —O—CH₂—, carbonyl, —NH—CO—, or —CO—NH-group,

wherein a hydrogen atom bound to a nitrogen atom may be replaced in each case by a C₁₋₃-alkyl group,

E^{b} denotes a phenylene group optionally substituted by a fluorine, chlorine, or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino, or C₁₋₃-alkoxy-carbonyl group, or

the group R^{c}-A^{c}-E^{c}-C₁₋₃-alkyl, wherein

R^{c} denotes a phenyl group optionally substituted by a fluorine, chlorine, or bromine atom, by a C₁₋₃-alkyl, trifluoromethyl, C₁₋₃-alkoxy, trifluoromethoxy, carboxy, or C₁₋₃-alkoxy-carbonyl group, or

a 5- to 7-membered cycloalkyleneimino group wherein

the cycloalkylene moiety may be fused to a phenyl ring, or

a hydrogen atom may be replaced by a C₁₋₃-alkyl group and/or

the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene, or 1,2-ethylenedioxy group,

A^{c} denotes a bond,

E^{c} denotes a 5-membered heteroarylene group bound via two carbon atoms which contains

an imino group optionally substituted by a C₁₋₃-alkyl group, an oxygen or sulfur atom,

an imino group optionally substituted by a C₁₋₃-alkyl group or an oxygen or sulfur atom and additionally a nitrogen atom, or

an imino group optionally substituted by a C₁₋₃-alkyl group and two nitrogen atoms, or

an oxygen or sulfur atom and two nitrogen atoms,

or a pyridinylene, pyridazinylene, or pyrimidinylene group,

while the abovementioned 5- and 6-membered heteroarylene groups in the carbon skeleton may be substituted by a fluorine, chlorine, or bromine atom, by a C₁₋₃-alkyl, trifluoromethyl, C₁₋₃-alkoxy, trifluoromethoxy, amino, C₁₋₃-alkylamino, acetylamino, C₁₋₃-alkoxy-carbonyl, or cyano group,

or R⁶ and R⁷ together denote an n-alkylene bridge with 4 or 5 carbon atoms, wherein

a hydrogen atom may be replaced by a C₁₋₃-alkyl group and/or

a —CH₂—CH₂— group may be replaced by a 1,2-linked phenylene group optionally substituted by a phenyloxy or benzyl group, while

the phenyloxy or benzyl group in the aromatic moiety and the phenylene group may be substituted independently of one another by a fluorine, chlorine, or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino, C₁₋₃-alkoxy-carbonyl, or cyano group,

or the carbon atom in the 3 position of an n-pentylene group may be monosubstituted by a C₁₋₃-alkyl group terminally substituted by an amino, C₁₋₃-alkylamino, di-(C₁₋₃-alkyl)-amino, acetylamino, or N-(methyl)-acetylamino group, or a 5- to 7-membered cycloalkyleneimino group, or may be disubstituted by a phenyl group and a cyano group,

while the phenyl groups mentioned in the definition of the abovementioned groups may, unless otherwise stated, be substituted by a fluorine, chlorine, or bromine atom, by a C₁₋₃-alkyl group, by a trifluoromethyl, C₁₋₃-alkoxy, trifluoromethoxy, phenyl, amino, C₁₋₃-alkylamino, acetylamino, C₁₋₃-alkoxy-carbonyl, or cyano group,

the alkyl and alkoxy groups mentioned in the definition of the above groups or in the alkyl moieties contained in the groups of formula 1 defined above with more than two carbon atoms may be straight-chain or branched, unless otherwise specified,

the carboxy groups mentioned in the definition of the abovementioned groups may be replaced by a group which can be converted into a carboxy group in vivo or by a group which is negatively charged under physiological conditions, and/or

the amino and imino groups mentioned in the definition of the abovementioned groups may be substituted by a group which can be cleaved in vivo,

their tautomers, their diastereomers, their enantiomers, the mixtures and the salts thereof, but particularly

the compounds of the above general formula 1, wherein

X₁ denotes the group CR¹,

X₂ denotes the group CR²,

X₃ denotes the group CR³, and

X₄ denotes the group CR⁴, or

one of the groups X₁ to X₄ denotes a nitrogen atom and the remainder of the groups X₁ to X₄ denote three of the groups CR¹ to CR⁴,

while R¹, R², R³ and R⁴ in each case denote a hydrogen atom, or

one or two of the groups R¹ to R⁴ independently of one another each denote a fluorine, chlorine, or bromine atom, a C₁₋₃-alkyl group, a trifluoromethyl, amino, C₁₋₃-alkylamino, or di-(C₁₋₃-alkyl)-amino group and the remainder of the groups R¹ to R⁴ each represent a hydrogen atom,

while R⁴ additionally together with R⁵ may assume the meaning of a —(CH₂)ₙ-bridge wherein n denotes the number 1, 2, or 3, and

A^{a} denotes a bond, an oxygen atom, a —CH₂—, —(CH₂)₂—, —NH—, —N(C₁₋₃-alkyl)-, sulfonyl, or carbonyl group, or a —NH—CH₂—, —NH—CO—, —NH—SO₂— group linked to the group R^{a} in formula I via the carbon or sulfur atom,

while a heteroatom of group A^{a} is not linked to a nitrogen atom of a 5-membered heteroaryl group of the group R^{a},

R^{a} denotes a phenyl or pyridinyl group,

a pyrrolyl, furanyl, thienyl, pyrazolyl, imidazolyl, oxazolyl, isoxazolyl, or thiazolyl group bound via a carbon or nitrogen atom,

while a nitrogen atom of the pyrrolyl, pyrazolyl and imidazolyl group may be substituted by a C₁₋₃-alkyl group and the phenyl group as well as the abovementioned heteroaromatic groups in the carbon skeleton may be substituted by a fluorine, chlorine, or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, or cyano group,

a 5- to 7-membered cycloalkyleneimino group wherein

the methylene group in the 4 position of a 6-membered cycloalkyleneimino group may be substituted by a methyl group or may be replaced by an oxygen or sulfur atom or by an imino group optionally substituted by a C₁₋₃-alkyl group, or

in a piperidino group a —CH₂— group linked to the imino nitrogen atom may be replaced by a carbonyl group, or a —(CH₂)₂— group linked to the imino nitrogen atom may be replaced by a —CO—NR⁸— group, or a —(CH₂)₃— group linked to the imino nitrogen atom may be replaced by a —CO—NR⁸—CO— group,

while R⁸ denotes a hydrogen atom or a C₁₋₃-alkyl group,

R⁵ denotes a hydrogen atom or a C₁₋₃-alkyl group,

Het denotes a 2,4-linked pyrrolylene or imidazolylene group which are bound in each case via the 2 position to the adjacent carbonyl group of formula I, and

are substituted at a nitrogen atom by a C₁₋₃-alkyl group and in the carbon skeleton may be substituted by a C₁₋₃-alkyl group or a trifluoromethyl group,

R⁶ denotes a hydrogen atom or a C₁₋₃-alkyl group,

R⁷ denotes a C₁₋₄-alkyl group terminally substituted by a C₃₋₇-cycloalkyl group, while

a hydrogen atom in the 4 position of a cyclohexyl group may be replaced by a C₁₋₅-alkoxy, C₁₋₃-alkoxy-C₁₋₃-alkyl, phenyl-C₁₋₃-alkoxy-methyl, phenyl-C₁₋₃-alkylamino, phenyl-C₁₋₂-alkyl-carbonylamino, benzoylamino, phenylaminocarbonyl, phenyl-C₁₋₃-alkylaminocarbonyl, carboxy, or C₁₋₃-alkoxy-carbonyl group, or

in a cyclopentyl group one or two single bonds separated from each other and from position 1 by at least one bond may each be fused to a phenyl group, while in a bi- or tricyclic ring system thus formed the hydrogen atom bound to the saturated carbon atom in the 1 position may be replaced by a C₁₋₃-alkylaminocarbonyl or di-(C₁₋₃-alkyl)aminocarbonyl group, while terminal methyl groups may each be wholly or partly fluorinated,

a C₁₋₆-alkyl group optionally substituted by a C₃₋₅-cycloalkyl group which is substituted

by a phenyl, 1-naphthyl, 2-naphthyl, pyridinyl, pyrimidinyl, pyrrolyl, furanyl, thienyl, pyrazolyl, imidazolyl, oxazolyl, isoxazolyl, thiazolyl, or isothiazolyl group,

while a nitrogen atom of the pyrrolyl, pyrazolyl and imidazolyl group may be substituted by a C₁₋₃-alkyl or trifluoromethyl group and the phenyl group and the above-mentioned heteroaromatic groups in the carbon skeleton may be substituted by a fluorine, chlorine, or bromine atom, by a C₁₋₄-alkyl, trifluoromethyl, C₁₋₃-alkoxy, fluoromethoxy, difluoromethoxy, trifluoromethoxy, C₁₋₄-alkoxy-carbonylamino-C₁₋₃-alkyl, amino, C₁₋₃-alkylamino, di-(C₁₋₃-alkyl)amino, or cyano group,

a C₁₋₆-alkyl group substituted by a phenyl group and a carboxy or C₁₋₃-alkoxy-carbonyl group,

a phenyl-C₂₋₃-alkynylene-CH₂ group wherein a hydrogen atom of the methylene group may be replaced in the 1 position by a methyl group and independently thereof the phenyl moiety may be substituted by a fluorine, chlorine, or bromine atom, by a C₁₋₄-alkyl, trifluoromethyl, C₁₋₃-alkoxy, phenyl, or cyano group,

the group R^{b}-A^{b}-E^{b}-C₁₋₃-alkyl optionally substituted in the C₁₋₃-alkyl moiety by a methyl group, wherein

R^{b} denotes a phenyl group optionally substituted by a fluorine, chlorine, or bromine atom, by a C₁₋₃-alkyl, trifluoromethyl, hydroxy, C₁₋₃-alkoxy, fluoromethoxy, difluoromethoxy, trifluoromethoxy, carboxy, or C₁₋₃-alkoxy-carbonyl group,

a 5-membered heteroaryl group which

may be bound via a carbon atom or, if A^{b} a bond denotes, may also be bound via a nitrogen atom and contains

an imino group optionally substituted by a C₁₋₃-alkyl group, or an oxygen or sulfur atom,

an imino group optionally substituted by a C₁₋₃-alkyl group, or an oxygen or sulfur atom and additionally a nitrogen atom, or

an imino group optionally substituted by a C₁₋₃-alkyl group and two nitrogen atoms, or

an oxygen or sulfur atom and two nitrogen atoms,

a 6-membered heteroaryl group which contains one or two nitrogen atoms,

while the abovementioned heteroaryl groups in the carbon skeleton may be monosubstituted by a fluorine, chlorine, or bromine atom, by a C₁₋₃-alkyl, trifluoromethyl, phenyl, C₁₋₃-alkoxy, trifluoromethoxy, amino, C₁₋₃-alkylamino, di-(C₁₋₃-alkyl)-amino, or acetylamino group or, with the exception of 5-membered heteroaryl groups containing more than two heteroatoms, may also be disubstituted by a C₁₋₄-alkyl group and a substituent selected from fluorine, chlorine, bromine, C₁₋₃-alkyl, trifluoromethyl, phenyl, C₁₋₃-alkoxy and trifluoromethoxy,

a C₃₋₆-cycloalkyl group, while

the two hydrogen atoms of the methylene group in the 3 position of a cyclopentyl group or in the 3 or 4 position of a cyclohexyl group may be replaced by an n-butylene, n-pentylene, or 1,2-ethylenedioxy group,

a 5- to 7-membered cycloalkyleneimino group wherein

the cycloalkylene moiety may be fused to a phenyl ring, or a hydrogen atom may be replaced by a C₁₋₃-alkyl group and/or

in each case the carbon atom in the 4 position of a 6- or 7-membered cycloalkyleneimino group may be substituted by a 4- to 7-membered cycloalkyleneimino, phenyl, or 4-(C₁₋₃-alkyl)-1,2,4-triazol-3-yl group, or

the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene, or 1,2-ethylenedioxy group,

A^{b} denotes a bond, an oxygen atom, a —CH₂—, —NH—, —O—CH₂—, carbonyl-, —NH—CO—, or —CO—NH— group,

wherein a hydrogen atom bound to a nitrogen atom may be replaced in each case by a C₁₋₃-alkyl group,

E^{b} denotes a phenylene group optionally substituted by a fluorine, chlorine, or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino, or C₁₋₃-alkoxy-carbonyl group, or

the group R^{c}-A^{c}-E^{c}-C₁₋₃-alkyl-, wherein

R^{c} denotes a phenyl group optionally substituted by a fluorine, chlorine, or bromine atom, by a C₁₋₃-alkyl, trifluoromethyl, C₁₋₃-alkoxy, trifluoromethoxy, carboxy, or C₁₋₃-alkoxy-carbonyl group, or

a 5- to 7-membered cycloalkyleneimino group wherein

the cycloalkylene moiety may be fused to a phenyl ring, or

a hydrogen atom may be replaced by a C₁₋₃-alkyl group and/or

the two hydrogen atoms of the methylene group in the 3 position of a 5-membered cycloalkyleneimino group or in the 3 or 4 position of a 6- or 7-membered cycloalkyleneimino group may be replaced by an n-butylene, n-pentylene, or 1,2-ethylenedioxy group,

A^{c} denotes a bond,

E^{c} denotes a 5-membered heteroarylene group bound via two carbon atoms which contains

an imino group optionally substituted by a C₁₋₃-alkyl group, or an oxygen or sulfur atom,

an imino group optionally substituted by a C₁₋₃-alkyl group, or an oxygen or sulfur atom and additionally a nitrogen atom, or

an imino group optionally substituted by a C₁₋₃-alkyl group and two nitrogen atoms, or

an oxygen or sulfur atom and two nitrogen atoms,

or a pyridinylene, pyridazinylene, or pyrimidinylene group,

while the abovementioned 5- and 6-membered heteroarylene groups in the carbon skeleton may be substituted by a fluorine, chlorine, or bromine atom, by a C₁₋₃-alkyl, trifluoromethyl, C₁₋₃-alkoxy, trifluoromethoxy, amino, C₁₋₃-alkylamino, acetylamino, C₁₋₃-alkoxy-carbonyl, or cyano group,

or R⁶ and R⁷ together denote an n-alkylene bridge with 4 or 5 carbon atoms wherein

a hydrogen atom may be replaced by a C₁₋₃-alkyl group and/or

a —CH₂—CH₂— group may be replaced by a 1,2-linked phenylene group optionally substituted by a phenyloxy or benzyl group, while

the phenyloxy or benzyl group in the aromatic moiety and the phenylene group may be substituted independently of one another by a fluorine, chlorine, or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino, C₁₋₃-alkoxy-carbonyl, or cyano group,

or the carbon atom in the 3 position of an n-pentylene group may be monosubstituted by a C₁₋₃-alkyl group terminally substituted by an amino, C₁₋₃-alkylamino, di-(C₁₋₃-alkyl)-amino, acetylamino, or N-(methyl)-acetylamino group, or a 5- to 7-membered cycloalkyleneimino group or may be disubstituted by a phenyl group and a cyano group,

while the phenyl groups mentioned in the definition of the abovementioned groups may, unless otherwise stated, be substituted by a fluorine, chlorine, or bromine atom, by a C₁₋₃-alkyl group, by a trifluoromethyl, C₁₋₃-alkoxy, trifluoromethoxy, phenyl, amino, C₁₋₃-alkylamino, acetylamino, C₁₋₃-alkoxy-carbonyl, or cyano group,

the alkyl and alkoxy groups mentioned in the definition of the above groups or in the alkyl moieties contained in the groups of formula I defined above with more than two carbon atoms may be straight-chain or branched, unless otherwise specified,

the carboxy groups mentioned in the definition of the abovementioned groups may be replaced by a group which can be converted into a carboxy group in vivo or by a group which is negatively charged under physiological conditions, and/or

the amino and imino groups mentioned in the definition of the abovementioned groups may be substituted by a group which can be cleaved in vivo,

their tautomers, their diastereomers, their enantiomers, the mixtures and the salts thereof.

Most particularly preferred compounds of the above general formula I are those wherein

X₁ denotes the group CR¹,

X₂ denotes the group CR²,

X₃ denotes the group CR³, and

X₄ denotes the group CR⁴,

while R¹, R², R³ and R⁴ in each case denote a hydrogen atom, or

one of the groups R¹ to R⁴ denotes a fluorine, chlorine, or bromine atom, a C₁₋₃-alkyl group or a trifluoromethyl group and the remainder of the groups R¹ to R⁴ in each case denote a hydrogen atom,

A^{a} denotes a bond, an oxygen atom, a —CH₂—, —(CH₂)₂—, —NH—, or —N(C₁₋₃-alkyl)-group,

while a nitrogen atom of the group A^{a} is not linked to a nitrogen atom of a 5-membered heteroaryl group of the group R ,

R^{a} denotes a phenyl, 2-pyridinyl, 3-pyridinyl, or 4-pyridinyl group,

a 1-pyrrolyl, 2-pyrrolyl, 3-pyrrolyl, 2-thienyl, or 3-thienyl group,

while the nitrogen atom of the pyrrolyl group may be substituted by a C₁₋₃-alkyl group and the phenyl group and the abovementioned heteroaromatic groups in the carbon skeleton may be substituted by a fluorine, chlorine, or bromine atom, by a C₁₋₃-alkyl or trifluoromethyl group,

a pyrrolidino, piperidino, or morpholino group

R⁵ denotes a hydrogen atom,

Het denotes a 2,4-linked pyrrolylene or imidazolylene group which are bound in each case via the 2 position to the adjacent carbonyl group of formula I, and

are substituted by a C₁₋₃-alkyl group at a nitrogen atom and may be substituted in the carbon skeleton by a C₁₋₃-alkyl group or a trifluoromethyl group,

R⁶ denotes a hydrogen atom or a C₁₋₃-alkyl group,

R⁷ denotes the group R^{d}—CH₂— or R^{d}—CH₂—CH₂—, wherein a hydrogen atom of the methylene group may be replaced in the 1 position by a C₁₋₃-alkyl group or a cyclopropyl group and wherein

R^{d} denotes a phenyl, 1-naphthyl, 2-naphthyl, 2-pyridinyl, 3-pyridinyl, 4-pyridinyl, 2-pyrimidinyl or 5-pyrimidinyl group,

while the phenyl group and the abovementioned heteroaromatic groups in the carbon skeleton may be substituted by a fluorine, chlorine, or bromine atom, by a C₁₋₄-alkyl, trifluoromethyl, C₁₋₃-alkoxy, or fluoromethoxy group,

a phenyl-C≡C—CH₂— group wherein a hydrogen atom of the methylene group in the 1 position may be replaced by a methyl group and independently thereof the phenyl moiety may be substituted by a fluorine, chlorine, or bromine atom, by a C₁₋₄-alkyl, trifluoromethyl, or phenyl group,

the group R^{b}-A^{b}-E^{b}-CH₂, wherein a hydrogen atom of the methylene group may be replaced in the 1 position by a methyl group and wherein

R^{b} denotes a phenyl group optionally substituted by a fluorine, chlorine, or bromine atom, by a C₁₋₃-alkyl, trifluoromethyl, hydroxy, methoxy, carboxy, or methoxycarbonyl group,

a pyrrolyl, pyrazolyl, imidazolyl, oxazolyl, isoxazolyl, thiazolyl, isothiazolyl, oxadiazole, or thiadiazolyl group bound via a carbon atom or, if A^{b} denotes a bond, also bound via a nitrogen atom, wherein a hydrogen atom bound to a nitrogen atom may be replaced by a C₁₋₃-alkyl group,

a 2-pyridyl, 3-pyridyl, 4-pyridyl, pyrazinyl, 2-pyrimidinyl, 4-pyrimidinyl, 5-pyrimidinyl, 3-pyridazinyl, or 4-pyridazinyl group,

while the abovementioned 5- and 6-membered heteroaryl groups in the carbon skeleton may be monosubstituted by a fluorine, chlorine, or bromine atom, by a C₁₋₃-alkyl, trifluoromethyl, phenyl, amino, C₁₋₃-alkylamino, di-(C₁₋₃-alkyl)-amino, or acetylamino group or, with the exception of 5-membered heteroaryl groups containing more than two heteroatoms, may also be disubstituted by a C₁₋₃-alkyl group and a substituent selected from fluorine, chlorine, bromine, C₁₋₃-alkyl, trifluoromethyl, phenyl,

a C₅₋₆-cycloalkyl group, while

the two hydrogen atoms of the methylene group in the 3 position of the cyclopentyl group or in the 4 position of the cyclohexyl group may be replaced by an n-butylene, n-pentylene, or 1,2-ethylenedioxy group,

or a 5- to 6-membered cycloalkyleneimino group wherein

the cycloalkylene moiety may be fused to a phenyl ring optionally substituted by a fluorine, chlorine, or bromine atom, by a C₁₋₃-alkyl, trifluoromethyl, or C₁₋₃-alkoxy group, or

a hydrogen atom may be replaced by a C₁₋₃-alkyl group and/or

the two hydrogen atoms of the methylene group in the 3 position of the 5-membered cycloalkyleneimino group or in the 4 position of the 6-membered cycloalkyleneimino group may be replaced by an n-butylene, n-pentylene, or 1,2-ethylenedioxy group,

A^{b} denotes a bond, a —CH₂—, —NH—, —O—CH₂—, —NH—CO—, or —CO—NH— group,

wherein a hydrogen atom bound to a nitrogen atom may be replaced in each case by a methyl group,

E^{b} denotes a 1,4-linked phenylene group, optionally substituted by a fluorine, chlorine, or bromine atom, by a C₁₋₃-alkyl, trifluoromethyl, C₁₋₃-alkoxy, or trifluoromethoxy group, or

the group R^{c}-A^{c}-E^{c}-C₁₋₃-alkyl-, wherein

R^{c} denotes a phenyl group optionally substituted by a fluorine, chlorine, or bromine atom, by a C₁₋₃-alkyl, trifluoromethyl, methoxy, carboxy, or methoxycarbonyl group,

A^{c} denotes a bond,

E^{c} denotes a pyrrolylene, pyrazolylene, imidazolylene, oxazolylene, isoxazolylene, thiazolylene, isothiazolylene, [1,3,4]-oxadiazolene, or [1,3,4]-thiadiazolene group bound via two carbon atoms in the relative positions 1,3, wherein a hydrogen atom bound to a nitrogen atom may be replaced by a C₁₋₃-alkyl group,

or a 1,4-linked pyridinylene, pyridazinylene, or pyrimidinylene group,

while the abovementioned 5- and 6-membered heteroarylene groups may be substituted in the carbon skeleton by a fluorine, chlorine, or bromine atom, by a C₁₋₃-alkyl, trifluoromethyl, or methoxy group,

while the alkyl and alkoxy groups mentioned in the definition of the above groups or in the alkyl moieties contained in the groups of formula I defined above with more than two carbon atoms may be straight-chain or branched, unless otherwise specified,

the carboxy groups mentioned in the definition of the abovementioned groups may be replaced by a group which can be converted into a carboxy group in vivo or by a group which is negatively charged under physiological conditions, and/or

the amino and imino groups mentioned in the definition of the abovementioned groups may be substituted by a group which can be cleaved in vivo,

their tautomers, their diastereomers, their enantiomers, the mixtures and the salts thereof.

The following are mentioned as examples of particularly preferred compounds:

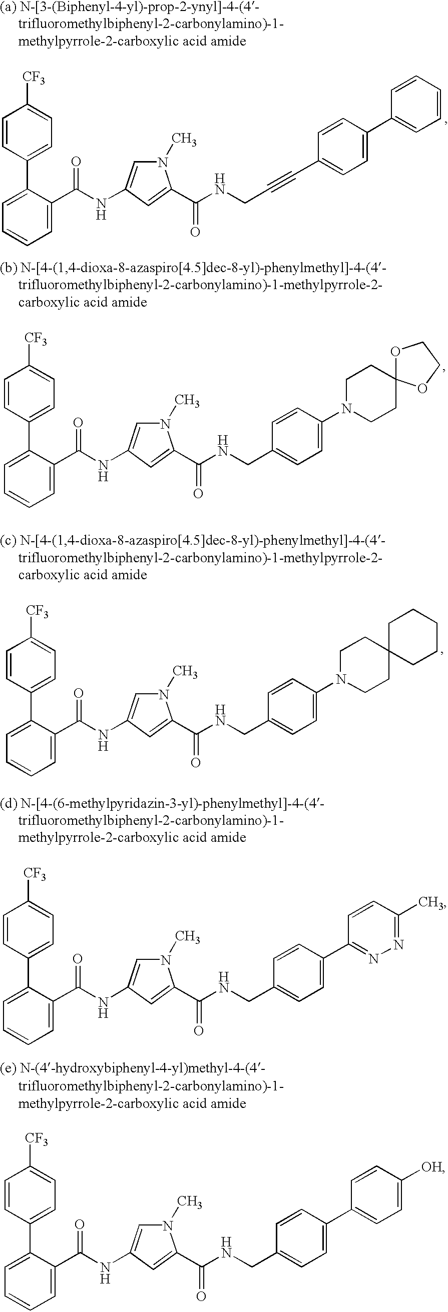

as well as their salts.

### DETAILED DESCRIPTION OF THE INVENTION

According to the invention the new compounds may be obtained by methods known from the literature, for example, by the following methods:

a. Reacting a Compound of General Formula

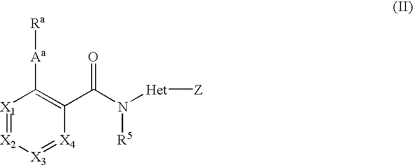

wherein:

X₁ to X₄, R^{a}, A^{a}, R⁵, and Het are as hereinbefore defined and Z denotes a carboxy group or a reactive derivative of a carboxy group,

with an amine of general formula

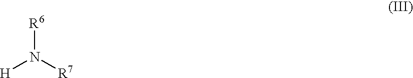

wherein R⁶ and R⁷ are as hereinbefore defined.

The reaction is expediently carried out with a corresponding halide or anhydride of general formula II in a solvent such as methylene chloride, chloroform, carbon tetrachloride, ether, tetrahydrofuran, dioxane, benzene, toluene, acetonitrile, or sulfolane optionally in the presence of an inorganic or organic base at temperatures between −20° C. and 200° C., but preferably at temperatures between −10° C. and 160° C. It may, however, also be carried out with the free acid, optionally in the presence of an acid-activating agent, e.g., propanephosphonic acid cycloanhydride or 2-(1H-benzotriazol-1-yl)-1,1,3,3-tetramethyluroniumtetrafluoroborate (TBTU), or a dehydrating agent, e.g., in the presence of isobutyl chloroformate, thionyl chloride, trimethylchlorosilane, hydrogen chloride, sulfuiric acid, methanesulfonic acid, p-toluenesulfonic acid, phosphorus trichloride, phosphorus pentoxide, N,N′-dicyclohexylcarbodiimide, N,N′-dicyclohexylcarbodiimide/N-hydroxysuccinimide, 1-hydroxybenzotriazole, N,N′-carbonyldiimidazole, N,N′-thionyldiimidazole, or triphenylphosphine/carbon tetrachloride, at temperatures between −20° C. and 200° C., but preferably at temperatures between −10° C. and 160° C.

b. Reacting a Compound of General Formula

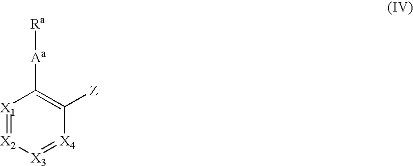

wherein X₁ to X₄, R^{a}, and A¹ are as hereinbefore defined and Z denotes a carboxy group or a reactive derivative of a carboxy group,

with an amine of general formula

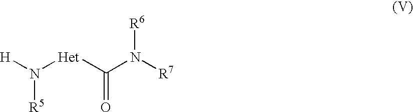

wherein R⁵ to R⁷ and Het are as hereinbefore defined.

The reaction may be carried out under the conditions specified above for method (a).

If according to the invention a compound of general formula I is obtained which contains an amino, alkylamino, or imino group, this may be converted by acylation or sulfonylation into a corresponding acyl or sulfonyl compound of general formula I, or

if a compound of general formula I is obtained which contains an amino, alkylamino, or imino group, this may be converted by alkylation or reductive alkylation into a corresponding alkyl compound of general formula I, or

if a compound of general formula I is obtained which contains a carboxy group this may be converted by esterification into a corresponding ester of general formula I, or

if a compound of general formula I is obtained which contains a carboxy or ester group, this may be converted by amidation into a corresponding amide of general formula I, or

if a compound of general formula I is obtained which contains an olefinic double bond or a C—C— triple bond, this may be converted by catalytic hydrogenation into a corresponding alkyl or alkylene compound of general formula I.

The subsequent acylation or sulfonylation is optionally carried out in a solvent or mixture of solvents such as methylene chloride, dimethylformamide, benzene, toluene, chlorobenzene, tetrahydrofuran, benzene/tetrahydrofuran, or dioxane with a corresponding acyl or sulfonyl derivative, optionally in the presence of a tertiary organic base or in the presence of an inorganic base or in the presence of a dehydrating agent, e.g., isobutyl chloroformate, thionyl chloride, trimethylchlorosilane, sulfuric acid, methanesulfonic acid, p-toluenesulfonic acid, phosphorus trichloride, phosphorus pentoxide, N,N′-dicyclohexylcarbodiimide, N,N′-dicyclohexylcarbodiimide/N-hydroxysuccinimide, or 1-hydroxybenzotriazole, and optionally additionally in the presence of 4-dimethylaminopyridine, N,N′-carbonyldiimidazole, or triphenylphosphine/carbon tetrachloride, conveniently at temperatures between 0° C. and 150° C., preferably at temperatures between 0° C. and 80° C.

The subsequent alkylation is optionally carried out in a solvent or mixture of solvents such as methylene chloride, dimethylformamide, benzene, toluene, chlorobenzene, tetrahydrofuran, benzene/tetrahydrofuran, or dioxane with an alkylating agent such as a corresponding halide or sulfonic acid ester, e.g., with methyl iodide, ethyl bromide, dimethylsulfate, or benzyl chloride, optionally in the presence of a tertiary organic base or in the presence of an inorganic base, conveniently at temperatures between 0° C. and 150° C., preferably at temperatures between 0° C. and 100° C.

The subsequent reductive alkylation is carried out with a corresponding carbonyl compound such as formaldehyde, acetaldehyde, propionaldehyde, acetone, or butyraldehyde in the presence of a complex metal hydride such as sodium borohydride, lithium borohydride, or sodium cyanoborohydride conveniently at a pH of 6 to 7 and at ambient temperature or in the presence of a hydrogenation catalyst, e.g., with hydrogen in the presence of palladium/charcoal, under a hydrogen pressure of 1 bar to 5 bar. The methylation is however preferably carried out in the presence of formic acid as reducing agent at elevated temperatures, e.g., at temperatures between 60° C. and 120° C.

The subsequent esterification is optionally carried out in a solvent or mixture of solvents such as methylene chloride, dimethylformamide, benzene, toluene, chlorobenzene, tetrahydrofuran, benzene/tetrahydrofuran, or dioxane or particularly advantageously in a corresponding alcohol, optionally in the presence of an acid such as hydrochloric acid or in the presence of a dehydrating agent, e.g., isobutyl chloroformate, thionyl chloride, trimethylchlorosilane, sulfuric acid, methanesulfonic acid, p-toluenesulfonic acid, phosphorus trichloride, phosphorus pentoxide, N,N′-dicyclohexylcarbodiimide, N,N′-dicyclohexylcarbodiimide/N-hydroxysuccinimide, or 1-hydroxybenzotriazole, and optionally additionally in the presence of 4-dimethylaminopyridine, N,N′-carbonyldiimidazole, or triphenylphosphine/carbon tetrachloride, conveniently at temperatures between 0° C. and 150° C., preferably at temperatures between 0° C. and 80° C.

The subsequent amidation is carried out by reacting a corresponding reactive carboxylic acid derivative with a corresponding amine optionally in a solvent or mixture of solvents such as methylene chloride, dimethylformamide, benzene, toluene, chlorobenzene, tetrahydrofuran, benzene/tetrahydrofuran, or dioxane, while the amine used may simultaneously serve as solvent, optionally in the presence of a tertiary organic base or in the presence of an inorganic base or with a corresponding carboxylic acid in the presence of a dehydrating agent, e.g., isobutyl chloroformate, thionyl chloride, trimethylchlorosilane, sulfuric acid, methanesulfonic acid, p-toluenesulfonic acid, phosphorus trichloride, phosphorus pentoxide, O-(benzotriazol-1-yl)-N,N,N′,N′-tetramethyluroniumtetrafluoroborate, N,N′-dicyclohexylcarbodiimide, N,N′-dicyclohexylcarbodiimide/N-hydroxysuccinimide, or 1-hydroxybenzotriazole, and optionally additionally in the presence of 4-dimethylaminopyridine, N,N′-carbonyldiimidazole, or triphenylphosphine/carbon tetrachloride, conveniently at temperatures between 0° C. and 150° C., preferably at temperatures between 0° C. and 80° C.

The subsequent catalytic hydrogenation is carried out with hydrogen in the presence of a catalyst such as palladium/charcoal or platinum in a solvent such as methanol, ethanol, ethyl acetate, dimethylformamide, dimethylformamide/acetone, or glacial acetic acid, optionally with the addition of an acid such as hydrochloric acid at temperatures between 0° C. and 50° C., but preferably at ambient temperature, and under a hydrogen pressure of 1 bar to 7 bar, but preferably from 3 bar to 5 bar.

In the reactions described hereinbefore, any reactive groups present such as hydroxy, carboxy, amino, alkylamino, or imino groups may be protected during the reaction by conventional protecting groups which are cleaved again after the reaction.

For example, a protecting group for a hydroxy group may be a trimethylsilyl, tert-butyldimethylsilyl, acetyl, benzoyl, methyl, ethyl, tert-butyl, trityl, benzyl, or tetrahydropyranyl group,

a protecting group for a carboxyl group may be a trimethylsilyl, methyl, ethyl, tert-butyl, benzyl, or tetrahydropyranyl group, and

protecting groups for an amino, alkylamino, or imino group may be a formyl, acetyl, trifluoroacetyl, ethoxycarbonyl, tert-butoxycarbonyl, benzyloxycarbonyl, benzyl, methoxybenzyl, or 2,4-dimethoxybenzyl group and additionally, for the amino group, a phthalyl group.

Any protecting group used is optionally subsequently cleaved for example by hydrolysis in an aqueous solvent, e.g., in water, isopropanol/water, acetic acid/water, tetrahydrofuran/water, or dioxane/water, in the presence of an acid such as trifluoroacetic acid, hydrochloric acid, or sulfuric acid, or in the presence of an alkali metal base such as sodium hydroxide or potassium hydroxide or aprotically, e.g., in the presence of iodotrimethylsilane, at temperatures between 0 and 120° C., preferably at temperatures between 10° C. and 100° C. However, a silyl group may also be cleaved using tetrabutylammonium fluoride as described hereinbefore.

However, a benzyl, methoxybenzyl, or benzyloxycarbonyl group is cleaved for example hydrogenolytically, e.g., with hydrogen in the presence of a catalyst such as palladium/charcoal in a suitable solvent such as methanol, ethanol, ethyl acetate, or glacial acetic acid, optionally with the addition of an acid such as hydrochloric acid at temperatures between 0° C. and 100° C., but preferably at temperatures between 20° C. and 60° C., and at a hydrogen pressure of 1 bar to 7 bar, but preferably 3 bar to 5 bar. A 2,4-dimethoxybenzyl group, however, is preferably cleaved in trifluoroacetic acid in the presence of anisole.

A tert-butyl or tert-butyloxycarbonyl group is preferably cleaved by treating with an acid such as trifluoroacetic acid or hydrochloric acid or by treating with iodotrimethylsilane, optionally using a solvent such as methylene chloride, dioxane, methanol, or diethyl ether.

A trifluoroacetyl group is preferably cleaved by treating with an acid such as hydrochloric acid, optionally in the presence of a solvent such as acetic acid at temperatures between 50° C. and 120° C. or by treating with sodium hydroxide solution, optionally in the presence of a solvent such as tetrahydrofuran at temperatures between 0° C. and 50° C.

A phthalyl group is preferably cleaved in the presence of hydrazine or a primary amine such as methylamine, ethylamine, or n-butylamine in a solvent such as methanol, ethanol, isopropanol, toluene/water, or dioxane at temperatures between 20° C. and 50° C.

Moreover, the compounds of general formula I obtained may be resolved into their enantiomers and/or diastereomers, as mentioned hereinbefore. Thus, for example, cis/trans mixtures may be resolved into their cis and trans isomers, and compounds with at least one optically active carbon atom may be separated into their enantiomers.

Thus, for example, the cis/trans mixtures may be resolved by chromatography into the cis and trans isomers thereof, the compounds of general formula I obtained which occur as racemates may be separated by methods known per se (cf N. L. Allinger and E. L. Eliel in “Topics in Stereochemistry”, Vol. 6, Wiley Interscience, 1971) into their optical antipodes and compounds of general formula I with at least 2 asymmetric carbon atoms may be resolved into their diastereomers on the basis of their physical-chemical differences using methods known per se, e.g., by chromatography and/or fractional crystallization, and, if these compounds are obtained in racemic form, they may subsequently be resolved into the enantiomers as mentioned above.

The enantiomers are preferably separated by column separation on chiral phases or by recrystallization from an optically active solvent or by reacting with an optically active substance which forms salts or derivatives such as e.g., esters or amides with the racemic compound, particularly acids and the activated derivatives or alcohols thereof, and separating the diastereomeric mixture of salts or derivatives thus obtained, e.g., on the basis of their differences in solubility, whilst the free antipodes may be released from the pure diastereomeric salts or derivatives by the action of suitable agents. Optically active acids in common use are e.g., the D- and L-forms of tartaric acid or dibenzoyltartaric acid, di-o-tolyltartaric acid, malic acid, mandelic acid, camphorsulfonic acid, glutamic acid, aspartic acid, or quinic acid. An optically active alcohol may be, for example, (+) or (−)-menthol and an optically active acyl group in amides, for example, may be a (+)-or (−)-menthyloxycarbonyl.

Furthermore, the compounds of formula I obtained may be onverted into the salts thereof, particularly for pharmaceutical use into the physiologically acceptable salts with inorganic or organic acids. Acids which may be used for this purpose include for example hydrochloric acid, hydrobromic acid, sulfuric acid, phosphoric acid, fumaric acid, succinic acid, lactic acid, citric acid, tartaric acid, or maleic acid.

Moreover, if the new compounds of formula I thus obtained contain an acidic group such as a carboxy group, they may subsequently, if desired, be converted into the salts thereof with inorganic or organic bases, particularly for pharmaceutical use into the physiologically acceptable salts thereof. Suitable bases for this purpose include for example sodium hydroxide, potassium hydroxide, arginine, cyclohexylamine, ethanolamine, diethanolamine and triethanolamine.

The compounds of general formulae II to V used as starting materials are known from the literature in some cases or may be obtained by methods known from the literature or are described in the Examples.

A compound of general formula II is obtained, for example, by reacting a compound of general formula

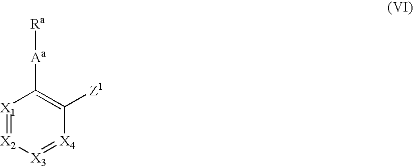

wherein X₁ to X₄, A^{a} and R^{a} are as hereinbefore defined and Z¹ denotes a carboxy group or a reactive derivative of a carboxy group, with an amine of general formula

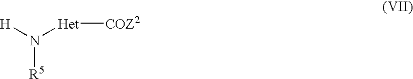

wherein R⁵ and Het are as hereinbefore defined and Z² denotes a protecting group for a carboxy group, and subsequently cleaving the protecting group.

The amines of general formula III are known from the literature or may be obtained by methods known from the literature.

The aromatic or heteroaromatic carboxylic acids according to general formula IV are known from the literature or may be obtained by methods known from the literature from corresponding aryl or heteroaryl educts.

The amino-heteroarylcarboxylic acid amides according to general formula V are also known from the literature or may easily be obtained from optionally substituted amino-heteroarylcarboxylic acids by reacting with the corresponding amines or from nitro-heteroarylcarboxylic acids by reacting with the corresponding amines and subsequently reducing the nitro group.

Starting compounds of formula V′, wherein Het denotes a 5-membered heteroarylene group which contains an imino group substituted by the group R⁹, while R⁹ together with R⁶ denotes a —(CH₂)ₚ— bridge, is obtained for example by the following synthesis plan:

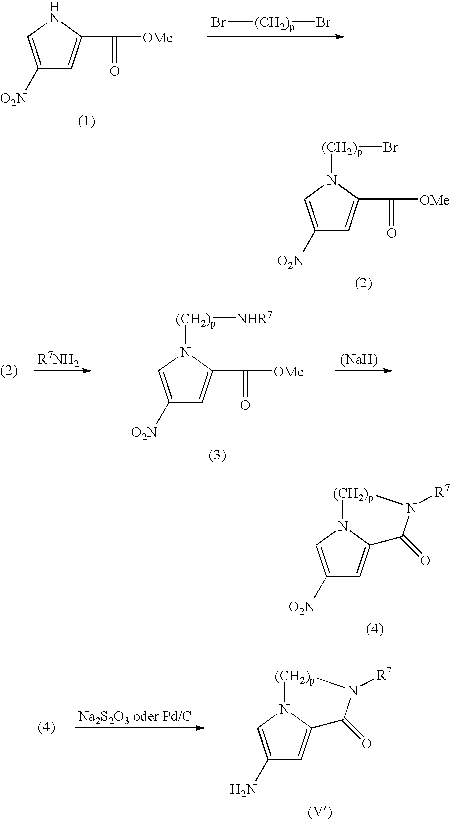

As already mentioned hereinbefore, the compounds of general formula I and the physiologically acceptable salts thereof have valuable pharmacological properties. In particular, they are valuable inhibitors of the microsomal triglyceride-transfer protein (MTP) and are therefore suitable for lowering the plasma levels of the atherogenic lipoproteins.

For example, the compounds according to the invention were investigated for their biological effects as follows:

Inhibitors of MTP were identified by a cell-free MTP activity kit. Solubilized liver microsomes from various species (e.g., rat or pig) could be used as the MTP source. To prepare donor and acceptor vesicles, lipids dissolved in organic solvents were mixed in suitable proportions and applied in a thin layer to the wall of a glass container by blowing the solvent in a nitrogen current. The solution used to prepare donor vesicles contained 400 μM phosphatidylcholine, 75 μM cardiolipin, and 10 μM [¹⁴C]-triolein (68.8 μCi/mg). To prepare acceptor vesicles, a solution of 1.2 mM phosphatidylcholine, 5 μM triolein and 15 μM [³H]-dipalmitoylphosphatidylcholine (108 mCi/mg) was used. Vesicles are formed by wetting the dried lipids with test buffer and then subjecting to ultrasound. Vesicle populations of uniform size were obtained by gel filtration of the ultrasonicated lipids. The MTP activity test contains donor vesicles, acceptor vesicles and the MTP source in test buffer. Substances were added from concentrated DMSO-containing stock solutions; the final concentration of DMSO in the test was 0.1%. The reaction was started by the addition of MTP. After a suitable incubation period, the transfer process was stopped by the addition of 500 μL of a SOURCE 30Q anion exchanger suspension (Pharmacia Biotech). The mixture was shaken for 5 minutes and the donor vesicles bound to the anion exchanger material were separated off by centrifuging. The radioactivity of [³H] and [¹⁴C] found in the supernatant was determined by liquid scintillation measurement and from this the recovery of the acceptor vesicles and the triglyceride transfer rate were calculated. The compounds of general formula I exhibit IC₅₀ values of ≦100 μM in the test described.

In view of the abovementioned biological properties the compounds of general formula I and the physiologically acceptable salts thereof are particularly suitable for lowering the plasma concentration of atherogenic apolipoprotein B (apoB)-containing lipoproteins such as chylomicrons and/or very low density lipoproteins (VLDL) as well as the residues thereof such as low density lipoproteins (LDL) and/or lipoprotein(a) (Lp(a)), for treating hyperlipidemias, for preventing and treating atherosclerosis and the clinical sequelae thereof, and for preventing and treating related disorders such as diabetes mellitus, adiposity and pancreatitis, oral administration being preferred.

The daily dose needed to achieve such an effect is between 0.5 mg and 500 mg, expediently between 1 mg and 350 mg, but preferably between 5 mg and 200 mg, in adults.

For this purpose, the compounds of formula I prepared according to the invention, optionally combined with other active substances such as other lipid-lowering agents, for example HMG-CoA-reductase inhibitors, cholesterol biosynthesis inhibitors such as squalene synthase inhibitors and squalene cyclase inhibitors, bile acid-binding resins, fibrates, cholesterol resorption inhibitors, niacin, probucol, CETP inhibitors and ACAT inhibitors may be incorporated together with one or more inert conventional carriers and/or diluents, e.g., with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose, or fatty substances such as hard fat or suitable mixtures thereof into conventional galenic preparations such as plain or coated tablets, capsules, powders, suspensions, or suppositories.

The Examples that follow are intended to illustrate the invention.

### EXAMPLE 1

N-[4-(3-methyl-5-phenylpyrazol-1-yl)phenylmethyl]-2-(biphenyl-2-carbonylamino)thiazole-4-carboxylic Acid Amide

a. 4-(3-methyl-5-phenylpyrazol-1-yl)benzonitrile

A solution of 20.0 g (0.118 mol) of 4-cyanophenylhydrazine and 19.1 g (0.118 mol) of benzoylacetone in 600 mL of methanol is combined with 16.7 mg triethylamine and stirred for two days. The solvent is distilled off, the residue taken up in dichloromethane, washed with water and dried with sodium sulfate. Then the mixture is chromatographed on a silica gel column, eluting with dichloromethane. Yield: 22.2 g (73% of theory); R_{f} value: 0.9 (silica gel; dichloromethane/methanol=19:1), C₁₇H₁₃N₃ (259.31); mass spectrum: (M+H)⁺=260.

b. 4-(3-methyl-5-phenylpyrazol-1-ylphenylmethylamine

22.2 g (0.086 mol) of 4-(3-methyl-5-phenylpyrazol-1-yl)benzonitrile are dissolved in 660 mL of methanolic ammonia and after the addition of Raney nickel hydrogenated at ambient temperature with hydrogen (3 bar). The catalyst is filtered off and the solution is concentrated by evaporation. The residue is chromatographed on silica gel, eluting with dichloromethane/methanol=4:1. Yield: 22 g (97% of theory); R_{f} value: 0.2 (silica gel; dichloromethane/methanol=9:1); C₁₇H₁₇N₃ (263.35); mass spectrum: (M+H)⁺=264; M⁺=263.

c. ethyl 2-aminothiazole-4-carboxylate

7.2 g (0.094 mol) of thiourea are dissolved in 100 mL of ethanol, at ambient temperature combined with 12.0 g (0.086 mol) of ethyl bromopyroracemate and then refluxed for 1.5 hours. After cooling, the mixture is diluted with 50 mL of water, made alkaline with concentrated ammonia and the precipitate is suction filtered. Yield: 12.5 g (84% of theory); R_{f} value: 0.5 (silica gel; dichloromethane/ethanol=19:1); C₆H₈N₂O₂S (172.21); mass spectrum: (M−H)⁻=171; (M+H)⁺=173; (M+Na)⁺=195.

d. ethyl 2-(biphenyl-2-carbonylamino)thiazole-4-carboxylate

1.0 g (5.0 mmol) of 2-biphenylcarboxylic acid are placed in 15 mL of dimethylformamide and after the addition of 0.9 g (5.45 mmol) of ethyl 2-aminothiazole-4-carboxylate, 1.8 g (5.60 mmol) of O-(benzotriazol-1-yl)-N,N′,N′-tetramethyluronium tetrafluoroborate (TBTU) and 2.9 mL (15.4 mmol) of N-ethyldiisopropylamine the mixture is stirred for 12 hours. The solution is concentrated by evaporation and chromatographed on silica gel, eluting with petroleum ether/ethyl acetate (10-30%). Yield: 0.5 g (28% of theory); R_{f} value: 0.3 (silica gel; petroleum ether/ethyl acetate=7:3); C₁₉H₁₆N₂O₃S (352.41); mass spectrum: (M+H)⁻=351; (M+Na)⁺=375.

e. 2-(Biphenyl-2-carbonylamino)thiazole-4-carboxylic Acid

0.5 g (1.4 mmol) of ethyl 2-(biphenyl-2-carbonylamino)thiazole-4-carboxylate are stirred in 30 mL of ethanol and 1.6 mL of 2 molar sodium hydroxide solution for 18 hours at ambient temperature. The solvent is distilled off, the residue is combined with water and acidified with 2 molar hydrochloric acid. The product precipitated is suction filtered. Yield: 0.3 g (72% of theory); R_{f} value: 0.4 (silica gel; dichloromethane/ethanol=4:1); C₁₇H₁₂N₂O₃S (324.36); mass spectrum: (M−H)⁻=323.

f. N-[4-(3-methyl-5-phenylpyrazol-1-yl)phenylmethyl]-2-(biphenyl-2-carbonylamino)thiazole-4-carboxylic Acid Amide

Prepared analogously to Example 1d from 2-(biphenyl-2-carbonylamino)thiazole-4-carboxylic acid, 4-(3-methyl-5-phenylpyrazol-1-yl)benzylamine, TBTU and N-ethyldiisopropylamine in dimethylformamide. Yield: 23% of theory; R_{f} value: 0.60 (silica gel; dichloromethane/ethanol 9:1); C₃₄H₂₇N₅O₂S (569.69); mass spectrum: (M−H)⁻=568; (M+Na)⁺=592.

### EXAMPLE 2

N-(biphenyl-4-ylmethyl-2-(biphenyl-2-carbonylaminothiazole-4-carboxylic Acid Amide

Prepared analogously to Example 1d from 2-(biphenyl-2-carbonylamino)thiazole-4-carboxylic acid, 4-phenylbenzylamine, TBTU and N-ethyldiisopropylamine in dimethylformamide. Yield: 86% of theory; R_{f} value: 0.40 (silica gel; dichloromethane/ethanol=19:1); C₃₀H₂₃N₃O₂S (489.60); mass spectrum: (M−H)⁻=488.

### EXAMPLE 3

N-(4-benzoylaminophenylmethyl)-2-(biphenyl-2-carbonylaminothiazole-4-carboxylic Acid Amide

Prepared analogously to Example 1d from 2-(biphenyl-2-carbonylamino)thiazole-4-carboxylic acid, 4-benzoylaminobenzylamine, TBTU and N-ethylduIsopropylamine in dimethylformamide. Yield: 25% of theory; R_{f} value: 0.60 (silica gel; dichloromethane/ethanol=9:1); C₃₁H₂₄N₄O₃S (532.62); mass spectrum: (M−H)⁻=531; (M+H)⁺=533; (M+Na)⁺=555.

### EXAMPLE 4

N-(biphenyl-4-ylmethyl-5-(4′-trifluoromethylbiphenyl-2-carbonylaminothiopbene-2-carboxylic Acid Amide

a. N-(biphenyl-4-yl)methyl-5-nitrothiophene-2-carboxylic Acid Amide

A mixture of 766 mg (4.0 mmol) of 5-nitrothiophene-2-carboxylic acid chloride, 733 mg (4.0 mmol) of 4-phenylbenzylamine and 1 mL of triethylamine are stirred in 45 mL of tetrahydrofuran for 18 hours. The solvent is distilled off and chromatographed on silica gel, eluting with dichloromethane. Yield: 540 mg (40% of theory); R_{f} value: 0.30 (silica gel; dichloromethane); C₁₈H₁₄N₂O₃S (338.39); mass spectrum: (M−H)⁻337.

b. N-(biphenyl-4-yl)methyl-5-aminothiophene-2-carboxylic Acid Amide

500 mg (1.47 mmol) of N-(biphenyl-4-yl)methyl-5-nitrothiophene-2-carboxylic acid amide are dissolved in 35 mL of methanol and 15 mL of dichloromethane and after the addition of 300 mg Raney nickel hydrogenated at ambient temperature with hydrogen (3 bar). The catalyst is filtered off and the solution concentrated by evaporation. Yield: 400 mg (88% of theory); R_{f} value: 0.30 (silica gel; dichloromethane/ethanol=50:1).

c. N-(biphenyl-4-ylmethyl-5-(4′-trifluoromethylbiphenyl-2-carbonylamino)thiophene-2-carboxylic Acid Amide

Prepared analogously to Example 4a from N-(biphenyl-4-yl)methyl-5-aminothiophene-2-carboxylic acid amide, 4′-trifluoromethylbiphenyl-2-carboxylic acid chloride and triethylamine in tetrahydrofuran. Yield: 43% of theory; R_{f} value: 0.50 (silica gel; dichloromethane/ethanol=19:1); C₃₂H₂₃F₃N₂O₂S (556.61); mass spectrum: (M−H)⁻555.

### EXAMPLE 5

N-[4-(3,4-dihydro-2H-quinolin-1-yl)phenylmethyl]-6-(4′-trifluoromethylbiphenyl-2-carbonylamino)pyrimidine-4-carboxylic Acid Amide

a. 4-(3.4-dihydro-2H-quinolin-1-yl)benzonitrile

5.3 g (0.04 mol) of 1,2,3,4-tetrahydroquinoline are dissolved in 60 mL of dimethylsulfoxide, 7.1 g (0.064 mol) of potassium tert-butoxide are added and the mixture is stirred for 20 minutes. Then 7.7 g (0.064 mol) of 4-fluorobenzonitrile in dimethylsulfoxide are added dropwise and the mixture is stirred for three days at 90° C. The reaction mixture is poured onto saturated sodium chloride solution and extracted with ethyl acetate. The combined organic extracts are chromatographed on aluminium oxide, eluting with petroleum ether/dichloromethane 1:1. Yield: 4.5 g (48% of theory); R_{f} value: 0.30 (silica gel; dichloromethane/petroleum ether =1:1); C₁₆H₁₄N₂ (234.30); mass spectrum: (M−H)⁻233.

b. 4-(3.4-dihydro-2H-!quinolin-1-yl)benzylamine

Prepared analogously to Example 1b from 4-(3,4-dihydro-2H-quinolin-1-yl)benzonitrile, Raney nickel and methanolic ammonia with the addition of hydrogen. Yield: 88% of theory; R_{f} value: 0.20 (silica gel; dichloromethane/ethanol=19:1); C₁₆H₁₈N₂ (238.34); mass spectrum: (M+H)⁺=239.

c. N-[4-(3.4-dihydro-2H-quinolin-1-yl)phenylmethyl]-6-chloropyrimidine-4-carboxylic Acid Amide

Prepared analogously to Example 4a from 4-(3,4-dihydro-2H-quinolin-1-yl)benzylamine, 6-chloropyrimidine-4-carboxylic acid chloride and triethylamine in tetrahydrofuran. Yield: 69% of theory; R_{f} value: 0.70 (silica gel; dichloromethane/ethanol=50:1); C₁₂H₁₉CIN₄O (378.86); mass spectrum: (M−H)⁻=377/79 (chlorine isotope).

d. N-[4-(3,4-dihydro-2H-quinolin-1-yl)phenylmethyl]-6-(2,3-dimethoxy-phenylmethylamino)pyrimidine-4-carboxylic Acid Amide

300 mg (0.79 mmol) of N-[4-(3,4-dihydro-2H-quinolin-1-yl)phenylmethyl]-6-chloropyrimidine-4-carboxylic acid amide and 500 mg (3.0 mmol) of 2,4-dimethoxybenzylamine are stirred for two hours at 160° C. After cooling, the mixture is chromatographed on silica gel, eluting with dichloromethane. Yield: 380 mg (94% of theory); R_{f} value: 0.80 (silica gel; dichloromethane/ethanol=19:1); C₃₀H₃₁N₅O₃ (509.61); mass spectrum: (M−H)⁻508; (M+Na)⁺=532.

e. N-[4-(3.4-dihydro-2H-quinolin-1-vyl)phenylmethyl]-6-aminopyrimidine-4-carboxylic Acid Amide

350 mg (0.68 mmol) of N-[4-(3,4-dihydro-2H-quinolin-1-yl)phenylmethyl]-6-(2,3-dimethoxybenzylamino)pyrimidine-4-carboxylic acid amide are dissolved in 30 mL of dichloromethane and after the addition of 7 mL trifluoroacetic acid stirred for two days. The solvent is distilled off, the mixture is made alkaline with methanolic ammonia and chromatographed on silica gel, eluting with dichloromethane/ethanol=99:1. Yield: 130 mg (53% of theory); R_{f} value: 0.70 (silica gel; dichloromethane/ethanol=19:1); C₂₁H₂₁N₅O (359.43); mass spectrum: (M−H)⁻358.

f. N-[4-(3,4-dihydro-2H-quinolin-1-yl)phenylmethyl]-6-(4′-trifluoromethylbiphenyl-2-carbonylamino)pyrimidine-4-carboxylic Acid Amide

Prepared analogously to Example 4a from N-[4-(3,4-dihydro-2H-quinolin-1-yl)phenylmethyl]-6-aminopyrimidine-4-carboxylic acid amide, 4′-trifluoromethylbiphenyl-2-carboxylic acid chloride and triethylamine in tetrahydrofuran. Yield: 17% of theory; R_{f} value: 0.40 (silica gel; petroleum ether/ethyl acetate=2:1); C₃₅H₂₈F₃N₅O₂ (607.63); mass spectrum: M⁺=607; (M+Na)⁺=630.

### EXAMPLE 6

N-[4-(3,4-dihydro-1H-isoquinolin-2-yl)phenylmethyl]-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 4-(3,4-dihydro-1H-isoquinolin-2-yl)benzylamine, TBTU and triethylamine in tetrahydrofuran. Yield: 100% of theory; R_{f} value: 0.60 (silica gel; dichloromethane/ethanol=9:1); C₃₆H₃₁F₃N₄O₂ (608.67); mass spectrum: (M−H)⁺=609; (M−H)⁻=607; (M−HCOO)⁻=653.

### EXAMPLE 7

N-(4′-methylbiphenyl-4-ylmethyl-5-(4′-trifluoromethylbiphenyl-2-carbonylamino Nicotinic Acid Amide

Prepared analogously to Example 1d from 5-(4′-trifluoromethylbiphenyl-2-carbonylamino)nicotinic acid, 4′-methylbiphenyl-4-methylamine, TBTU and N-ethyldiisopropylamine in dimethylformamide. Yield: 26% of theory; R_{f} value: 0.49 (silica gel; dichloromethane/ethanol=9:1); C₃₄H₂₆F₃N₃O₂ (565.60); mass spectrum: (M−H)⁻564; (M+Na)⁺=588.

### EXAMPLE 8

N-(4-phenylaminocarbonylphenylmethyl)-5-(4′-trifluoromethylbiphenyl-2-carbonylamino)nicotinic Acid Amide

Prepared analogously to Example 1d from 4-phenylaminocarbonylbenzylamine, 5-(4′-trifluoromethylbiphenyl-2-carbonylamino)nicotinic acid, TBTU and N-ethyldiisopropylamine in dimethylformamide. Yield: 21% of theory; R_{f} value: 0.41 (silica gel; dichloromethane/ethanol=9:1); C₃₄H₂₅F₃N₄O₃ (594.59); mass spectrum: M⁺=594.

### EXAMPLE 9

N-[4-(3-methyl-5-phenylpyrazol-1-yl)phenylmethyl]-5-(4′-trifluoromethylbiphenyl-2-carbonylamino)nicotinic Acid Amide

Prepared analogously to Example 1d from 5-(4′-trifluoromethylbiphenyl-2-carbonylamino)nicotinic acid, 4-(3-methyl-5-phenylpyrazol-1-yl)benzylamine, TBTU and N-ethyldiisopropylamine in dimethylformamide. Yield: 32% of theory; R_{f} value: 0.48 (silica gel; dichloromethane/ethanol=9:1); C₃₇H₂₈F₃N₅O₂ (631.66); mass spectrum: (M+Na)⁺=654.

### EXAMPLE 10

N-(4′-methylbiphenyl-4-yl)methyl-4-(4′-trifluoromethylbiphenyl-2-carbonylamino)-1-methylimidazol-2-carboxylic Acid Amide

Prepared analogously to Example 1d from 4-(4′-trifluoromethylbiphenyl-2-carbonylamino)-1-methylimidazol-2-carboxylic acid, 4′-methylbiphenyl-4-methylamine, TBTU and N-ethyldiisopropylamine in dimethylformamide. Yield: 10% of theory; R_{f} value: 0.95 (silica gel; dichloromethane/ethanol=4:1); C₃₃H₂₇F₃N₄O₂ (568.60); mass spectrum: (M−H)⁻567; (M+Na)⁺=591.

### EXAMPLE 11

N-(biphenyl-4-yl)methv1-4-(4′-trifluoromethylbi-henyl-2-carbonylamino)-1-methylimidazol-2-carboxylic Acid Amide

A solution of 100 mg (0.25 mmol) of 4-(4′-trifluoromethylbiphenyl-2-carbonylamino)-1-methylimidazol-2-carboxylic acid, 48 mg (0.25 mmol) of 4-phenylbenzylamine and 0.2 mL (1.5 mmol) of N-methylmorpholine in 6 mL of dichloromethane is combined with 0.3 mL (0.5 mmol) of propanephosphonic acid cycloanhydride (50 wt. % in ethyl acetate) at −10° C. and stirred for 2 hours with cooling. Then it is washed with 2 molar hydrochloric acid and 2 molar sodium hydroxide solution, the combined organic extracts are dried and concentrated by evaporation. Yield: 0.12 g (84% of theory); R_{f} value: 0.59 (silica gel; dichloromethane/ethanol=9:1); C₃₂H₂₅F₃N₄O₂ (554.57); mass spectrum: (M−H)⁻553; (M+H)⁺=555; (M+Na)⁺=577.

### EXAMPLE 12

N-[4-(piperidino)phenylmethyl]-4-(4′-trifluoromethylbiphenyl-2-carbonylamino)-1-methylimidazol-2-carboxylic Acid Amide

Prepared analogously to Example 11 from 4-(piperldino)benzylamine and 4-(4′-trifluoromethylbiphenyl-2-carbonylamino)-1-methylimidazole-2-carboxylic acid in dichloromethane with the addition of propanephosphonic acid cycloanhydride and N-methylmorpholine. Yield: 88% of theory; R_{f} value: 0.53 (silica gel; dichloromethane/ethanol=9:1); C₃₁H₃₀F₃N₅O₂ (561.61); mass spectrum: (M−H)⁻=560.

### EXAMPLE 13

N-[4-(3.4-dihydro-2H-quinolin-1-yl)phenylmethyl]-4-(4′-trifluoromethylbiphenyl-2-carbonylamino)-1-methylimidazol-2-carboxylic Acid Amide

Prepared analogously to Example 11 from 4-(3,4-dihydro-2H-quinolin-1-yl)benzylamine and 4-(4′-trifluoromethylbiphenyl-2-carbonylamino)-1-methylimidazole-2-carboxylic acid in dichloromethane with the addition of propanephosphonic acid cycloanhydride and N-methylmorpholine. Yield: 85% of theory; R_{f} value: 0.71 (silica gel; dichloromethane/ethanol 9:1); C₃₅H₃₀F₃N₅O₂ (609.65); mass spectrum: (M−H)⁻608.

### EXAMPLE 14

N-(4′-trifluoromethybiphenyl-4-ylmethyl-4-(4′-trifluoromethylbiphenyl-2-carbonylamino)-1-methylimidazol-2-carboxylic Acid Amide

Prepared analogously to Example 11 from 4′-trifluoromethylbiphenyl-4-methylamine and 4-(4′-trifluoromethylbiphenyl-2-carbonylamino)-1-methylimidazol-2-carboxylic acid in dichloromethane with the addition of propanephosphonic acid cycloanhydride and N-methylmorpholine.

Yield: 83% of theory; R_{f} value: 0.52 (silica gel; dichloromethane/ethanol=9:1); C₃₃H₂₄F₆N₄O₂ (622.57); mass spectrum: (M−H)⁻621.

### EXAMPLE 15

N-(4′-Chlorobiphenyl-4-yl)methyl-4-(4′-trifluoromethylbiphenyl-2-carbonylamino)-1-methylimidazol-2-carboxylic Acid Amide

Prepared analogously to Example 11 from 4′-chlorobiphenyl-4-methylamine and 4-(4′-trifluoromethylbiphenyl-2-carbonylamino)-1-methylimidazole-2-carboxylic acid in dichloromethane with the addition of propanephosphonic acid cycloanhydride and N-methylmorpholine.

Yield: 88% of theory; R_{f} value: 0.54 (silica gel; dichloromethane/ethanol=9:1); C₃₂H₂₄ClF₃N₄O₂ (589.02); mass spectrum: (M−H)⁻587/89 (chlorine isotope).

### EXAMPLE 16

N-[4-(ipridin-4-yl)phenylmethyl]-4-(4′-trifluoromethylbiphenyl-2-carbonylamino)-1-methylimidazole-2-carboxylic Acid Amide

Prepared analogously to Example 11 from 4-(pyridin-4-yl)benzylamine and 4-(4′-trifluoromethylbiphenyl-2-carbonylamino)-1-methylimidazole-2-carboxylic acid in dichloromethane with the addition of propanephosphonic acid cycloanhydride and N-methylmorpholine.

Yield: 94% of theory; R_{f} value: 0.41 (silica gel; dichloromethane/ethanol=9:1); C₃₁H₂₄F₃N₅O₂ (555.56); mass spectrum: (M−H)⁻554.

### EXAMPLE 17

N-[4-([1.2.3]-thiadiazol-4-yl)phenylmethyl]-4-(4′-trifluoromethylbiphenyl-2-carbonylamino)-1-methylimidazole-2-carboxylic Acid Amide

Prepared analogously to Example 11 from 4-([1,2,3]-thiadiazol-4-yl)benzylamine and 4-(4′-trifluoromethylbiphenyl-2-carbonylamino)-1-methylimidazole-2-carboxylic acid in dichloromethane with the addition of propanephosphonic acid cycloanhydride and N-methylmorpholine.

Yield: 88% of theory; R_{f} value: 0.52 (silica gel; dichloromethane/ethanol=9:1); C₂₈H₂₁F₃N₆O₂S (562.57); mass spectrum: (M−H)⁻=561.

### EXAMPLE 18

N-[4-(6-methylpyridazin-3-yl)phenylmethyl]-4-(4′-trifluoromethylbiphenyl-2-carbonylamino)-1-methylimidazole-2-carboxylic Acid Amide

a. 4-(6-methylpyridazin-3-yl)benzonitrile

875 mg (6.8 mmol) of 3-chloro-6-methylpyridazine and 237 mg (0.2 mmol) of tetrakis(triphenylphosphine)palladium(0) are placed in 40 mL of toluene, a solution of 1.0 g (6.8 mmol) of 4-cyanophenylboric acid in 20 mL of methanol and 1.4 g (13.6 mmol) of sodium carbonate in 20 mL of water are added and the mixture is refluxed for 7 hours. The reaction mixture is stirred for two days at ambient temperature and concentrated by evaporation. The residue is chromatographed on silica gel, eluting with dichloromethane/ethanol=9:1. Yield: 340 mg (26% of theory); R_{f} value: 0.53 (silica gel; dichloromethane/ethanol=9:1); C 12H₉N₃ (195.23); mass spectrum: (M+H)⁺=196.

b. 4-(6-methylpyridazin-3-yl)benzylamine

Prepared analogously to Example 1b from 4-(6-methylpyridazin-3-yl)benzonitrile and Raney nickel in methanolic ammonia with the addition of hydrogen (3 bar). Yield: 73% of theory; R_{f} value: 0.13 (silica gel; dichloromethane/ethanol=75:25); C₁₂H₁₃N₃ (199.26); mass spectrum: (M+H)⁺=200.

c. N-[4-(6-methylpyridazin-3-yl)phenylmethyl]-4-(4′-trifluoromethylbiphenyl-2-carbonylamino)-1-methylimidazole-2-carboxylic Acid Amide

Prepared analogously to Example 11 from 4-(6-methylpyridazin-3-yl)benzylamine and 4-(4′-trifluoromethylbiphenyl-2-carbonylamino)-1-methylimidazole-2-carboxylic acid in dichloromethane with the addition of propanephosphonic acid cycloanhydride and N-methylmorpholine.

Yield: 96% of theory; R_{f} value: 0.51 (silica gel; dichloromethane/ethanol=9:1); C₃₁H₂₅F₃N₆O₂ (570.57); mass spectrum: (M−H)⁻=569; (M+H)⁺=571; (M+Na)⁺=593.

### EXAMPLE 19

N-[3-(4-biphenyl)prop-2-ynyl]-4-(4′-trifluoromethylbiphenyl-2-carbonylamino)-1-methylimidazole-2-carboxylic Acid Amide

a. N-tert-butoxycarbonylprop-2-ynylamine

6.9 g (0.12 mol) of propargylamine is placed in 50 mL of dichloromethane, at 0° C. a solution of 27.3 g (0.12 mol) of di-tert-butyldicarbonate in 50 mL of dichloromethane is added dropwise and the mixture is stirred for three hours at ambient temperature. Then it is cooled to −20° C. and the product precipitated is suction filtered. Yield: 18.2 g (94% of theory).

b. N-tert-butoxycarbonyl-3-(4-biphenyl)prop-2-ynylamine

A mixture of 1.3 g (5.3 mmol) of 4-bromobiphenyl, 0.1 g (0.53 mmol) of copper(I) iodide, 0.6 g (0.53 mmol) of tetrakis(triphenylphosphine)palladium(0) and 2.2 mL (16.1 mmol) of triethylamine are refluxed in 30 mL of tetrahydrofuran for 10 minutes, then the mixture is combined with 1.0 g (6.4 mmol) of N-tert-butoxycarbonylprop-2-ynylamine and refluxed for a further 10 hours. The precipitate is filtered off and the filtrate is concentrated by evaporation. The residue is chromatographed on silica gel, eluting with petroleum ether/ethyl acetate 96:4. Yield: 370 mg (22% of theory); R_{f} value: 0.62 (silica gel; petroleum ether/ethyl acetate=7:3); C₂₀H₂₁NO₂ (307.4); mass spectrum: (M+Na)⁺=330.

c. 3-(4-biphenyl)-prop-2-ynylaminetrifluoroacetate

365 mg (1.1 mmol) of N-tert-butoxycarbonyl-3-(4-biphenyl)prop-2-ynylamine are stirred for 2 hours in 20 mL of dichloromethane and 2 mL of trifluoroacetic acid. Then it is concentrated by evaporation and the residue is reacted further directly. Yield: 381 mg (quantitative); R_{f} value: 0.22 (silica gel; dichloromethane/ethanol=9:1).

d. N-[3-(4-biphenyl)-prop-2-ynyl]-4-(4′-trifluoromethylbiphenyl-2-carbonylamino)-1-methylimidazole-2-carboxylic Acid Amide

Prepared analogously to Example 11 from 3-biphenyl-4-ylprop-2-ynylamine-trifluoroacetate and 4-(4′-trifluoromethylbiphenyl-2-carbonylamino)-1-methylimidazole-2-carboxylic acid in dichloromethane with the addition of propanephosphonic acid cycloanhydride and N-methylmorpholine.

Yield: 58% of theory; R_{f} value: 0.59 (silica gel; dichloromethane/ethanol 9:1); C₃₄H₂₅F₃N₄O₂ (578.59); mass spectrum: (M−H)⁻577; (M+H)⁺=579; (M+Na)⁺=601.

### EXAMPLE 20

N-(4′-hydroxybiphenyl-4-yl)methyl-4-(4′-trifluoromethylbiphenyl-2-carbonylamino)-1-methylimidazole-2-carboxylic Acid Amide

Prepared analogously to Example 11 from 4′-hydroxybiphenyl-4-methylamine and 4-(4′-trifluoromethylbiphenyl-2-carbonylamino)-1-methylimidazole-2-carboxylic acid in dichloromethane with the addition of propanephosphonic acid cycloanhydride and N-methylmorpholine.

Yield: 30% of theory; R_{f} value: 0.45 (silica gel; dichloromethane/ethanol=9:1); C₃₂H₂₅F₃N₄O₃ (570.57); mass spectrum: (M−H)⁻569.

### EXAMPLE 21

N-[3-(4-trifluoromethylphenyl)prop-2-ynyl]-4-(4′-trifluoromethylbiphenyl-2-carbonyl-amino)-1-methylimidazole-2-carboxylic Acid Amide

Prepared analogously to Example 11 from 3-(4-trifluoromethylphenyl)prop-2-ynylamine and 4-(4′-trifluoromethylbiphenyl-2-carbonylamino)-1-methylimidazole-2-carboxylic acid in dichloromethane with the addition of propanephosphonic acid cycloanhydride and N-methylmorpholine.

Yield: 71% of theory; R_{f} value: 0.49 (silica gel; dichloromethane/ethanol 9:1); C₂₉H₂₀F₆N₄O₂ (570.49); mass spectrum: (M−H)⁻569; (M+Na)⁺=593.

### EXAMPLE 22

N-[4-(1.4-dioxaspiro[4.5]dec-8-yl)phenylmethyl]-4-(4′-trifluoromethylbiphenyl-2-carbonylamino)-1-methylimidazole-2-carboxylic Acid Amide

Prepared analogously to Example 11 from 4-(1,4-dioxaspiro[4.5]dec-8-yl)benzylamine and 4-(4′-trifluoromethylbiphenyl-2-carbonylamino)-1-methylimidazole-2-carboxylic acid in dichloromethane with the addition of propanephosphonic acid cycloanhydride and N-methylmorpholine.

Yield: 67% of theory; R_{f} value: 0.62 (silica gel; dichloromethane/ethanol=9:1); C₃₄H₃₃F₃N₄O₄ (618.66); mass spectrum: (M−H)⁻=617.

### EXAMPLE 23

N-[3-(4-tert-butylphenyl)prop-2-ynyl]-4-(4′-trifluoromethylbiphenyl-2-carbonylamino)-1-methylimidazole-2-carboxylic Acid Amide

Prepared analogously to Example 11 from 3-(4-tert-butylphenyl)prop-2-ynylamine and 4-(4′-trifluoromethylbiphenyl-2-carbonylamino)-1-methylimidazole-2-carboxylic acid in dichloromethane with the addition of propanephosphonic acid cycloanhydride and N-methylmorpholine.

Yield: 33% of theory; R_{f} value: 0.52 (silica gel; dichloromethane/ethanol 9:1); C₃₂H₂₉F₃N₄O₂ (558.60); mass spectrum: (M−H)⁻=557; (M+Na)⁺=581.

### EXAMPLE 24

N-(4′-methylbiphenyl-4-yl)methyl-4-(4′-trifluoromethylbiphenyl-2-carbonylamino)-1-methylpyrrole-2-carboxylic Acid Amide

Prepared analogously to Example 1d from 4′-methylbiphenyl-4-methylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.40 (silica gel; dichloromethane/ethanol=19:1); C₃₄H₂₈F₃N₃O₂ (567.61); mass spectrum: (M−H)-=566; (M+Na)⁺=590.

### EXAMPLE 25

N-(4-phenylcarbonylaminophenylmethyl)-4-(4′-trifluoromethylbiphenyl-2-carbonylamino)-1-methylpyrrole-2-carboxylic Acid Amide

Prepared analogously to Example 1d from 4-phenylcarbonylaminobenzylamine, 4-(4′-trifluoromethylbiphenyl-2-carbonylamino)-1-methylpyrrole-2-carboxylic acid, TBTU and N-ethylduisopropylamine in dimethylformamide. Yield: 62% of theory; R_{f} value: 0.20 (silica gel; dichloromethane/ethanol=19:1); C₃₄H₂₇F₃N₄O₃ (596.61); mass spectrum: (M−H)⁻=595; (M+Na)⁺=619.

### EXAMPLE 26

N-[4-(3-methyl-5-phenylpyrazol-1-yl)phenylmethyl]-4-(4′-trifluoromethylbiphenyl-2-carbonylamino)-1-methylpyrrole-2-carboxylic Acid Amide

Prepared analogously to Example 1d from 4-(3-methyl-5-phenylpyrazol-1-yl)benzylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.25 (silica gel; dichloromethane/ethanol=19:1); C₃₇H₃₀F₃N₅O₂ (633.67); mass spectrum: (M−H)⁻=632; (M+Na)⁺=656.

### EXAMPLE 27

N-(4′-methylbiphenyl-4-yl)methyl-4-(biphenyl-2-carbonylamino)-1-methylpyrrole-2-carboxylic Acid Amide

Prepared analogously to Example 1d from 4′-methylbiphenyl-4-methylamine, 4-(biphenyl-2-carbonylamino)-1-methylpyrrole-2-carboxylic acid, TBTU and N-ethyldiisopropylamine in dimethylformamide. Yield: 99% of theory; R_{f} value: 0.40 (silica gel; dichloromethane/ethanol=19:1); C₃₃H₂₉N₃O₂ (499.61); mass spectrum: M⁺=499.

### EXAMPLE 28

N-benzyl-4-(4′-trifluoromethylbiphenyl-2-carbonylamino)-1-methylpyrrole-2-carboxylic Acid Amide

Prepared analogously to Example 1d from benzylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.60 (silica gel; dichloromethane/ethanol=9:1); C₂₇H₂₂F₃N₃O₂ (477.49); mass spectrum: (M−H)⁻476; (M+Na)⁺=490.

### EXAMPLE 29

N-pyridin-2-ylmethyl-4-(4′-trifluoromethylbiphenyl-2-carbonylamino)-1-methylpyrrole-2-carboxylic Acid Amide

Prepared analogously to Example 1d from 2-(aminomethyl)pyridine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.50 (silica gel; dichloromethane/ethanol=9:1); C₂₆H₂₁F₃N₄O₂ (478.47); mass spectrum: (M−H)⁻=477.

### EXAMPLE 30

N-pyridin-3-ylmethyl-4-(4′-trifluoromethylbiphenyl-2-carbonylamino)-1-methylpyrrole-2-carboxylic Acid Amide

Prepared analogously to Example 1d from 3-(aminomethyl)pyridine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.40 (silica gel; dichloromethane/ethanol 9:1); C₂₆H₂₁F₃N₄O₂ (478.47); mass spectrum: (M−H)-=477; (M+Na)⁺=501.

### EXAMPLE 31

N-pyridin-4-ylmethyl-4-(4′-trifluoromethylbiphenyl-2-carbonylamino)-1-methylpyrrole-2-carboxylic Acid Amide

Prepared analogously to Example 1d from 4-(aminomethyl)pyridine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.35 (silica gel; dichloromethane/ethanol 9:1); C₂₆H₂₁F₃N₄O₂ (478.47); mass spectrum: (M−H)⁻477; (M+Na)⁺=501.

### EXAMPLE 32

N-methoxycarbonylmethyl-4-(4′-trifluoromethylbiphenyl-2-carbonylamino)-1-methyl-pyrrole-2-carboxylic Acid Amide

Prepared analogously to Example 1d from glycine methyl ester hydrochloride, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.70 (silica gel; dichloromethane/ethanol=9:1); C₂₃H₂₀F₃N₃O₄ (459.42); mass spectrum: (M−H)⁻=458; (M+Na)⁺=482.

### EXAMPLE 33

N-(2-methoxycarbonyleth-1)-4-(4′-trifluoromethylbiphenyl-2-carbonylamino)-1-methyl-pyrrole-2-carboxylic Acid Amide

Prepared analogously to Example 1d from P-alaninemethylester hydrochloride, 4-(4′-trifluoromethylbiphenyl-2-carbonylamino)-1-methylpyrrole-2-carboxylic acid, TBTU and N-ethyldlisopropylamine in dimethylformamide. Yield: quantitative; R_{f} value: 0.70 (silica gel; dichloromethane/ethanol=9:1); C₂₄H₂₂F₃N₃O₄ (473.45); mass spectrum: (M−H)⁻472; (M+Na)⁺=496.

### EXAMPLE 34

N-(4-[1.2,3]-thiadiazol-4-ylphenylmethyl)-4-(4′-trifluoromethylbiphenyl-2-carbonylamino)-1-methylpyrrole-2-carboxylic Acid Amide

Prepared analogously to Example 1d from 4-[1,2,3]-thiadiazol-4-ylbenzylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.70 (silica gel; dichloromethane/ethanol=9:1); C₂₉H₂₂F₃N₅O₂S (561.59); mass spectrum: (M−H)⁻=560.

### EXAMPLE 35

N-[2-(4-methylphenyl)pyridine-5-ylmethyl]-4-(4′-trifluoromethylbiphenyl-2-carbonylamino)-1-methylpyrrole-2-carboxylic Acid Amide

Prepared analogously to Example 1d from (2-(4-methylphenyl)pyridin-5-yl)methylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.55 (silica gel; dichloromethane/ethanol=9:1); C₃₃H₂₇F₃N₄O₂ (568.60); mass spectrum: (M−H)⁻567; (M+Na)⁺=591.

### EXAMPLE 36

N-[4-(pidin-4-yl)phenylmethyl]-4-(4′-trifluoromethylbiphenyl-2-carbonylamino)-1-methylpyrrole-2-carboxylic Acid Amide

Prepared analogously to Example 1d from 4-(pyridin-4-yl)benzylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.45 (silica gel; dichloromethane/ethanol=9:1); C₃₂H₂₅F₃N₄O₂ (554.57); mass spectrum: (M−H)⁻=553.

### EXAMPLE 37

N-[4-(N-methyl-N-cyclohexylaminocarbonyl)phenylmethyl]-4-(4′-trifluoromethyl-biphenyl-2-carbonylamino)-1-methylpvrrole-2-carboxylic Acid Amide

Prepared analogously to Example 1d from 4-(N-methyl-N-cyclohexylaminocarbonyl)benzylamine, 4-(4′-trifluoromethylbiphenyl-2-carbonylamino)-1-methylpyrrole-2-carboxylic acid, TBTU and N-ethyldiisopropylamine in dimethylformamide. Yield: 98% of theory; R_{f} value: 0.7 (silica gel; dichloromethane/ethanol=9:1); C₃₅H₃₅F₃N₄O₃ (616.68); mass spectrum: (M−H)⁻615.

### EXAMPLE 38

N-(4-bromophenylmethyl)-4-(4′-trifluoromethylbiphenyl-2-carbonylamino)-1-methylpyrrole-2-carboxylic Acid Amide

Prepared analogously to Example 1d from 4-bromobenzylamine hydrochloride, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.7 (silica gel; dichloromethane/ethanol=9:1); C₂₇H₂₁BrF₃N₃O₂ (556.38); mass spectrum: (M−H)⁻554/56 (bromine isotope).

### EXAMPLE 39

N-(4′-trifluoromethylbiphenyl-4-yl)methyl-4-(4′-trifluoromethylbiphenyl-2-carbonylamino)-1-methylpyrrole-2-carboxylic Acid Amide

Prepared analogously to Example 1d from 4′-trifluoromethylbiphenyl-4-methylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; Rf value: 0.7 (silica gel; dichloromethane/ethanol=9:1); C₃₄H₂₅F₆N₃O₂ (621.58); mass spectrum: (M−H)⁻620.

### EXAMPLE 40

N-(4′-chlorobiphenyl-4-ylmethyl-4-(4′-trifluoromethylbiphenyl-2-carbonylamino)-1-methylpyrrole-2-carboxylic Acid Amide

Prepared analogously to Example 1d from 4′-chlorobiphenyl-4-methylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.7 (silica gel; dichloromethane/ethanol=9:1); C₃₃H₂₅ClF₃N₃O₂ (588.03); mass spectrum: (M−H)⁻586/88 (chlorine isotope).

### EXAMPLE 41

N-[3-(4-methylphenyl)prop-2-ynyl]-4-(4′-trifluoromethylbiphenyl-2-carbonylamino)-1-methylpyrrole-2-carboxylic Acid Amide

Prepared analogously to Example 1d from 3-(4-methylphenyl)prop-2-ynylamine, 4-(4′-trifluoromethylbiphenyl-2-earbonylamino)-1-methylpyrrole-2-carboxylic acid, TBTU and N-ethyldiisopropylamine in dimethylformamide. Yield: 57% of theory; R_{f} value: 0.6 (silica gel; dichloromethane/ethanol=9:1); C₃₀H₂₄F₃N₃O₂ (515.54); mass spectrum: (M−H)⁻=514.

### EXAMPLE 42

N-[3-(4-isopropylphenyl)prop-2-ynyl]-4-(4′-trifluoromethylbiphenyl-2-carbonylamino)-1-methylpyrrole-2-carboxylic Acid Amide

Prepared analogously to Example 1d from 3-(4-isopropylphenyl)prop-2-ynylamine, 4-(4′-trifluoromethylbiphenyl-2-carbonylamino)-1-methylpyrrole-2-carboxylic acid, TBTU and N-ethyldiisopropylamine in dimethylformamide. Yield: 82% of theory; R_{f} value: 0.7 (silica gel; dichloromethane/ethanol=9:1); C₃₂H₂₈F₃N₃O₂ (543.59); mass spectrum: (M−H)⁻=542.

### EXAMPLE 43

N-hydroxycarbonylmethyl-4-(4′-trifluoromethylbiphenyl-2-carbonylamino)-1-methylpyrrole-2-carboxylic Acid Amide

Prepared analogously to Example 1e from N-methoxycarbonylmethyl-4-(4′-trifluoromethylbiphenyl-2-carbonylamino)-1-methylpyrrole-2-carboxylic acid amide and 2 molar sodium hydroxide solution in methanol. Yield: 77% of theory; R_{f} value: 0.3 (silica gel; dichloromethane/ethanol=4:1); C₂₂H₁₈F₃N₃O₄ (445.40); mass spectrum: (M−H)⁻=444; (M+Na)⁺=468.

### EXAMPLE 44

N-(2-hydroxycarbonylethyl)-4-(4′-trifluoromethylbiphenyl-2-carbonylamino)-1-methylpyrrole-2-carboxylic Acid Amide

Prepared analogously to Example 1e from N-(2-methoxycarbonylethyl)-4-(4′-trifluoromethylbiphenyl-2-carbonylamino)-1-methylpyrrole-2-carboxylic acid amide and 2 molar sodium hydroxide solution in methanol. Yield: 67% of theory; R_{f} value: 0.3 (silica gel; dichloromethane/ethanol 4:1); C₂₃H₂OF₃N₃O₄ (459.42); mass spectrum: (M−H)⁻458.

### EXAMPLE 45

N-(biphenyl-3-methyl)-4-(4′-trifluoromethylbiphenyl-2-carbonylamino)-1-methylpyrrole-2-carboxylic Acid Amide

Prepared analogously to Example 1d from 3-phenylbenzylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.8 (silica gel; dichloromethane/ethanol=9:1); C₃₃H₂₆F₃N₃O₂ (553.58); mass spectrum: (M−H)⁻=552.

### EXAMPLE 46

N-(2′-methylbiphenyl-4-yl)methyl-4-(4′-trifluoromethylbiphenyl-2-carbonylamino)-1-methylpyrrole-2-carboxylic Acid Amide

Prepared analogously to Example 1d from 2′-methylbiphenyl-4-methylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.75 (silica gel; dichloromethane/ethanol=9:1); C₃₄H₂₈F₃N₃O₂ (567.61); mass spectrum: (M−H)⁻=566.

### EXAMPLE 47

N-(4′-methoxycarbonylbiphenyl-4-yl)methyl-4-(4′-trifluoromethylbiphenyl-2-carbonylamino)-1-methylpyrrole-2-carboxylic Acid Amide

Prepared analogously to Example 1d from 4′-methoxycarbonylbiphenyl-4-methylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.75 (silica gel; dichloromethane/ethanol=9:1); C₃₅H₂₈F₃N₃O₄ (611.62); mass spectrum: (M−H)⁻=610.

### EXAMPLE 48

N-[4-(2iperidinophenylmethyl)-4-(4′-trifluoromethylbiphenyl-2-carbonylamino)-1-methylpyrrole-2-carboxylic Acid Amide

Prepared analogously to Example 1d from 4-(piperidino)benzylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.70 (silica gel; dichloromethane/ethanol=9:1); C₃₂H₃₁F₃N₄O₂ (560.62); mass spectrum: (M−H)⁻559.

### EXAMPLE 49

N-[4-(1.4-dioxaspiro[4.5]dec-8-yl)phenylmethyl]-4-(4′-trifluoromethylbiphenyl-2-carbonylamino)-1-methylpyrrole-2-carboxylic Acid Amide

Prepared analogously to Example 1d from 4-(1,4-dioxaspiro[4.5]dec-8-yl)benzylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.70 (silica gel; dichloromethane/ethanol=9:1); C₃₅H₃₄F₃N₃O₄ (617.67); mass spectrum: (M+Na)⁺=640.

### EXAMPLE 50

N-(4-tert-butylphenylmethyl)-4-(4′-trifluoromethylbiphenyl-2-carbonylamino)-1-methylpyrrole-2-carboxylic Acid Amide

Prepared analogously to Example 1d from 4-tert-butylbenzylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.70 (silica gel; dichloromethane/ethanol=9:1); C₃₁H₃₀F₃N₃O₂ (533.59).

### EXAMPLE 51

N-(4-chlorophenylmethyl)-4-(4′-trifluoromethylbiphenyl-2-carbonylamino)-1-methylpyrrole-2-carboxylic Acid Amide

Prepared analogously to Example 1d from 4-chlorobenzylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.70 (silica gel; dichloromethane/ethanol=9:1); C₂₇H₂₁ClF₃N₃O₂ (511.93); mass spectrum: (M−H)⁻=510/12 (chlorine isotope).

### EXAMPLE 52

N-(2-phenylthiazol-4-ylmethyl)-4-(4′-trifluoromethylbiphenyl-2-carbonylamino)-1-methylpyrrole-2-carboxylic Acid Amide

Prepared analogously to Example 1d from (2-phenylthiazol-4-yl)methylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.70 (silica gel; dichloromethane/ethanol=9:1); C₃₀H₂₃F₃N₄O₂S (560.60); mass spectrum: (M−H)⁻=559.

### EXAMPLE 53

N-(3-chloro-5-trifluoromethylpyridin-2-ylmethyl)-4-(4′-trifluoromethylbiphenyl-2-carbonylamino)-1-methylpyrrole-2-carboxylic Acid Amide

Prepared analogously to Example 1d from 3-chloro-5-trifluoromethylpyridin-2-ylmethylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.80 (silica gel; dichloromethane/ethanol=9:1); C₂₇H₁₉ClF₆N₄O₂ (580.92); mass spectrum: (M−H)⁻579/81 (chlorine isotope).

### EXAMPLE 54

N-(5-phenyl-[1.3.4]oxadiazol-2-ylmethyl)-4-(4′-trifluoromethylbiphenyl-2-carbonylamino)-1-methylpyrrole-2-carboxylic Acid Amide

Prepared analogously to Example 1d from (5-phenyl-[1,3,4]oxadiazol-2-yl)methylamine, 4-(4′-trifluoromethylbiphenyl-2-carbonylamino)-1-methylpyrrole-2-carboxylic acid, TBTU and N-ethyldiisopropylamine in dimethylformamide. Yield: 76% of theory; R_{f} value: 0.70 (silica gel; dichloromethane/ethanol=9:1); C₂₉H₂₂F₃N₅O₃ (545.52); mass spectrum: (M−H)⁻=544.

### EXAMPLE 55

N-[4-(pyrimidin-4-ylcarbonylaminophenylmethyl]-4-(4′-trifluoromethylbiphenyl-2-carbonylamino)-1-methylpyrrole-2-carboxylic Acid Amide

Prepared analogously to Example 1d from 4-(pyrimidin-4-ylcarbonylamino)benzylamine, 4-(4′-trifluoromethylbiphenyl-2-carbonylamino)-1-methylpyrrole-2-carboxylic acid, TBTU and N-ethyldiisopropylamine in dimethylformamide. Yield: 99% of theory; R_{f} value: 0.70 (silica gel; dichloromethane/ethanol=9:1); C₃₂H₂₅F₃N₆O₃ (598.58); mass spectrum: (M−H)⁻597.

### EXAMPLE 56

N-(biphenyl-4-yl)methyl-N-methyl-4-(4′-trifluoromethylbiiphenyl-2-carbonylamino)-1-methylpyrrole-2-carboxylic Acid Amide

Prepared analogously to Example 1d from N-methyl-4-phenylbenzylamine, 4-(4′-trifluoromethylbiphenyl-2-carbonylamino)-1-methylpyrrole-2-carboxylic acid, TBTU and N-ethyldiisopropylamine in dimethylformamide. Yield: 77% of theory; R_{f} value: 0.80 (silica gel; dichloromethane/ethanol=9:1); C₃₄H₂₈F₃N₃O₂ (567.61); mass spectrum: (M−H)⁻=566.

### EXAMPLE 57

N-[4-(3-4-dihydro-2H-quinolin-1-yl)phenylmethyl]-4-(4′-trifluoromethylbiphenyl-2-carbonylamino)-1-methylpyrrole-2-carboxylic Acid Amide

Prepared analogously to Example 1d from 4-(3,4-dihydro-2H-quinolin-1-yl)benzylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.65 (silica gel; dichloromethane/ethanol=9:1); C₃₆H₃₁F₃N₄O₂ (608.66); mass spectrum: (M−H)⁻=607.

### EXAMPLE 58

N-[4-(pyridin-3-yl)phenylmethyl]-4-(4′-trifluoromethylbiphenyl-2-carbonylamino)-1-methylpyrrole-2-carboxylic Acid Amide

Prepared analogously to Example 1d from 4-(pyridin-3-yl)benzylamine, 4-(4′-trifluoromethylbiphenyl-2-carbonylamino)-1-methylpyrrole-2-carboxylic acid, TBTU and N-ethyldiisopropylamine in dimethylformamide. Yield: 37% of theory; R_{f} value: 0.65 (silica gel; dichloromethane/ethanol=9:1); C₃₂H₂₅F₃N₄O₂ (554.57); mass spectrum: (M−H)-=553.

### EXAMPLE 59

N-(4′-methylbiphenyl-4-yl)methyl-4-(4′-fluorobiphenyl-2-carbonylamino)-1-methylpyrrole-2-carboxylic Acid Amide

Prepared analogously to Example 1d from 4′-methylbiphenyl-4-methylamine, 4-(4′-fluorobiphenyl-2-carbonylamino)-1-methylpyrrole-2-carboxylic acid, TBTU and N-ethyldiisopropylamine in dimethylformamide. Yield: 82% of theory; R_{f} value: 0.80 (silica gel; dichloromethane/ethanol -9:1); C₃₃H₂₈FN₃O₂ (517.60).

### EXAMPLE 60

N-(4′-methylbiphenyl-4-yl)methyl-4-(4′-methylbiphenyl-2-carbonylamino)-1-methylpyrrole-2-carboxylic Acid Amide

Prepared analogously to Example 1d from 4′-methylbiphenyl-4-methylamine, 4-(4′-methylbiphenyl-2-carbonylamino)-1-methylpyrrole-2-carboxylic acid, TBTU and N-ethyldiisopropylamine in dimethylformamide. Yield: quantitative; R_{f} value: 0.70 (silica gel; dichloromethane/ethanol=9:1); C₃₄H₃₁N₃O₂ (513.64); mass spectrum: (M−H)⁻=512.

### EXAMPLE 61

N-(4′-hydroxycarbonylbiphenyl-4-ylmethyl-4-(4′-trifluoromethylbiphenyl-2-carbonylamino)-1-methylpyrrole-2-carboxylic Acid Amide

Prepared analogously to Example 1e from N-(4′-methoxycarbonylbiphenyl-4-yl)methyl-4-(4′-trifluoromethylbiphenyl-2-carbonylamino)-1-methylpyrrole-2-carboxylic acid amide and 2 molar sodium hydroxide solution in ethanol. Yield: quantitative; R_{f} value: 0.40 (silica gel; dichloromethane/ethanol=9:1); C₃₄H₂₆F₃N₃O₄ (597.59); mass spectrum: (M−H)⁻=596.

### EXAMPLE 62

N-(4′-hydroxybiphenyl-4-yl)methyl-4-(4′-trifluoromethylbiphenyl-2-carbonylamino)-1-methylpyrrole-2-carboxylic Acid Amide

Prepared analogously to Example 1d from 4-(4-hydroxyphenyl)benzylamine, 4-(4′-trifluoromethylbiphenyl-2-carbonylamino)-1-methylpyrrole-2-carboxylic acid, TBTU and N-ethyldiisopropylamine in dimethylformamide. Yield: 58% of theory; R_{f} value: 0.50 (silica gel; dichloromethane/ethanol=9:1); C₃₃H₂₆F₃N₃O₃ (569.58); mass spectrum: (M−H)⁻=568.

### EXAMPLE 63

N-(4-methoxycarbonyl-4-phenylhexyl)-4-(4′-trifluoromethylbiphenyl-2-carbonylamino)-1-methylpyrrole-2-carboxylic Acid Amide

Prepared analogously to Example 1d from methyl 5-amino-2-ethyl-2-phenylpentanoate, 4-(4′-trifluoromethylbiphenyl-2-carbonylamino)-1-methylpyrrole-2-carboxylic acid, TBTU and N-ethyl-diisopropylamine in dimethylformamide. Yield: 21% of theory; R_{f} value: 0.40 (silica gel; petroleum ether/ethyl acetate=2:3); C₃₄H₃₄F₃N₃O₄ (605.66); mass spectrum: (M−H)⁻=604.

### EXAMPLE 64

N-(4′-methylbiphenyl-4-yl)methyl-4-(4′-trifluoromethylbiphenyl-2-carbonylamino)-1H-pyrrole-2-carboxylic Acid Amide

Prepared analogously to Example 11 from 4′-methylbiphenyl-4-methylamine and 4-(4′-trifluoromethylbiphenyl-2-carbonylamino)-1H-pyrrole-2-carboxylic acid in dichloromethane with the addition of propanephosphonic acid cycloanhydride and N-methylmorpholine. Yield: 17% of theory; R_{f} value: 0.58 (silica gel; dichloromethane/ethanol=9:1); C₃₃H₂₆F₃N₃O₂ (553.58); mass spectrum: (M−H)⁻552.

### EXAMPLE 65

N-(4′-methylbiphenyl-4-yl)methyl-4-(4′-trifluoromethylbiphenyl-2-carbonylamino)-1-ethylpyrrole-2-carboxylic Acid Amide

Prepared analogously to Example 1d from 4′-methylbiphenyl-4-methylamine, 4-(4′-trifluoromethylbiphenyl-2-carbonylamino)-1-ethylpyrrole-2-carboxylic acid, TBTU and N-ethyldiisopropylamine in dimethylformamide. Yield: 78% of theory; R_{f} value: 0.80 (silica gel; dichloromethane/ethanol=9:1); C₃₅H₃₀F₃N₃O₂ (581.64); mass spectrum: (M−H)⁻=580.

### EXAMPLE 66

N-[4-(6-methylpyridazin-3-ylphenylmethyl]-4-(4′-trifluoromethylbiphenyl-2-carbonylamino)-1-methylpyrrole-2-carboxylic Acid Amide

Prepared analogously to Example 1d from 4-(6-methylpyridazin-3-yl)benzylamine, 4-(4′-trifluoromethylbiphenyl-2-carbonylamino)-1-methylpyrrole-2-carboxylic acid, TBTU and N-ethyldiisopropylamine in dimethylformamide. Yield: 28% of theory; R_{f} value: 0.49 (silica gel; dichloromethane/ethanol=9:1); C₃₂H₂₆F₃N₅O₂ (569.59); mass spectrum: (M−H)⁻568; (M+H)⁺=570; (M+Na)⁺=592.

### EXAMPLE 67

N-[4-(pyridin-2-yl)1henylmethyl]-4-(4′-trifluoromethylbiphenyl-2-carbonylamino)-1-methylpyrrole-2-carboxylic Acid Amide

Prepared analogously to Example 1d from 4-(pyridin-2-yl)benzylamine, 4-(4′-trifluoromethylbiphenyl-2-carbonylamino)-1-methylpyrrole-2-carboxylic acid, TBTU and N-ethyldiisopropylamine in dimethylformamide. Yield: quantitative; R_{f} value: 0.55 (silica gel; dichloromethane/ethanol=9:1); C₃₂H₂₅F₃N₄O₂ (554.57); mass spectrum: (M−H)⁻553; (M+Na)⁺=577.

### EXAMPLE 68

N-[3-(4-methylphenyl)propyl]-4-(4′-trifluoromethylbiphenyl-2-carbonylamino)-1-methylpyrrole-2-carboxylic Acid Amide

50 mg (0.097 mmol) of N-[3-(4-methylphenyl)prop-2-ynyl]-4-(4′-trifluoromethylbiphenyl-2-carbonylamino)-1-methylpyrrole-2-carboxylic acid amide are dissolved in 10 mL of ethanol and after the addition of 20 mg palladium on activated charcoal (10%) hydrogenated with hydrogen. The catalyst is filtered off and the solution is concentrated by evaporation. Yield: 40 mg (79% of theory); R_{f} value: 0.35 (silica gel; petroleum ether/ethyl acetate=1:1); C₃₀H₂₈F₃N₃O₂ (519.57); mass spectrum: (M−H)⁻=518.

### EXAMPLE 69

N-(4′-methylbiphenyl-4-yl)methyl-4-[2-(moipholin-4-yl)phenylcarbonylamino]-1-methylpyrrole-2-carboxylic Acid Amide

a. ethyl 2-(morpholin-4-yl)benzoate

A mixture of 1.7 mL (10.6 mmol) of ethyl 2-bromobenzoate, 1.0 mL (11.0 mmol) of morpholine, 5.4 g (16.5 mmol) of cesium carbonate, 75 mg (0.33 mmol) of palladium-IT-acetate and 270 mg (0.43 mmol) of 2,2′-bis-(diphenylphosphino)-1,1′-binaphthyl are stirred in 30 mL xylene for 12 hours at 100° C. The solvent is distilled off and the residue is chromatographed on silica gel, eluting with dichloromethane/ethanol=9:1. Yield: 0.6 g (25% of theory); R_{f} value: 0.80 (silica gel; dichloromethane/ethanol=19:1); C₁₃H₁₇NO₃ (235.29); mass spectrum: (M+H)⁺=236; (M+Na)⁺=258.

b. 2-(morpholin-4-yl)benzoic Acid

Prepared analogously to Example 1e from ethyl 2-(morpholin-4-yl)benzoate and 2 molar sodium hydroxide solution in methanol. Yield: 90% of theory; R_{f} value: 0.75 (silica gel; dichloromethane/ethanol/ammonia=8:4:0.2); C₁₁H₁₃NO₃ (207.23); mass spectrum: (M−H)⁻=206; (M+H)⁺=208.

c. methyl 1-methyl-4-[2-(morpholin-4-yl)phenylcarbonylamino]pyrrole-2-carboxylate

0.2 g (0.89 mmol) of 2-(morpholin-4-yl)benzoic acid are stirred in 1.0 mL (13.7 mmol) of thionyl chloride with the addition of 2 drops of dimethylformamide for 90 minutes. The solution is concentrated by evaporation, 0.2 g (0.89 mmol) of methyl 1-methyl-4-aminopyrrole-2-carboxylate, 0.4 mL (2.7 mmol) of triethylamine and 20 mL of tetrahydrofuran are added and the mixture is stirred for 17 hours. The solvent is distilled off, the residue dissolved in dichloromethane and washed with water. The combined organic extracts are dried and concentrated by evaporation.

Yield: 0.3 g (100% of theory); R_{f} value: 0.35 (silica gel; dichloromethane/ethanol=19:1); C₁₈H₂₁N₃O₄ (343.39); mass spectrum: (M−H)⁻342; (M+Na)⁺=366.

d. 1-methyl-4-[2-(morpholin-4-yl)phenylcarbonylamino]pyrrole-2-carboxylic acid

Prepared analogously to Example 1e from methyl 1-methyl-4-[2-(morpholin-4-yl)phenylcarbonylamino]pyrrole-2-carboxylate and 2 molar sodium hydroxide solution in methanol. Yield: 75% of theory; R_{f} value: 0.60 (silica gel; dichloromethane/ethanol=9:1); C₁₇H₁₉N₃O₄ (329.36); mass spectrum: (M−H)⁻328; (M+Na)⁺=352.

e. N-(4′-methylbiphenyl-4-ylmethyl-4-[2-(morpholin-4-yl)phenylcarbonylamino]-1-methylpyrrole-2-carboxylic Acid Amide

Prepared analogously to Example 1e from 1-methyl-4-[2-(morpholin-4-yl)phenylcarbonylamino]pyrrole-2-carboxylic acid, 4′-methylbiphenyl-4-methylamine, TBTU and N-ethyldiisopropylamine in dimethylformamide. Yield: 94% of theory; R_{f} value: 0.55 (silica gel; dichloromethane/ethanol=9:1); C₃₁H₃₂N₄O₃ (508.62); mass spectrum: (M−H)⁻=507.

### EXAMPLE 70

N-(4′-methylbiphenyl-4-yl)methyl-4-(4′-trifluoromethylbiphenYl-2-carbonylamino)-1-(3-tert-butoxycarbonylaminopropyl)pyrrole-2-carboxylic Acid Amide

Prepared analogously to Example 1d from 4′-trifluoromethylbiphenyl-2-carboxylic acid and N-(4′-methylbiphenyl-4-yl)methyl-4-amino-1-(3-tert-butoxycarbonylaminopropyl)pyrrole-2-carboxylic acid amide, TBTU and N-ethyldiisopropylamine in dimethylformamide. Yield: quantitative; R_{f} value: 0.60 (silica gel; dichloromethane/ethanol=9:1); C₄₁H₄₁F₃N₄O₄ (710.80); mass spectrum: (M−H)⁻=709; (M+Na)⁺=733.

### EXAMPLE 71

N-(4-benzyloxybenzyl)-N-methyl-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, N-(4-benzyloxybenzyl)methylamine, TBTU and N-ethyldiisopropylamine in dimethylformamide. Yield: 79% of theory; R_{f} value: 0.54 (silica gel; petroleum ether/ethyl acetate=1:2); C₃₅H₃₀F₃N₃O₃ (597.64); mass spectrum: (M−H)⁻596; (M+H)⁺=598.

### EXAMPLE 72

N-[4-(2-methoxycarbonylethyl)phenylmethyl]-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 4-(2-methoxycarbonylethyl)benzylamine, TBTU and triethylamine in tetrahydrofuran. Yield: 85% of theory; R_{f} value: 0.78 (silica gel; dichloromethane/ethanol=9:1); C₃₁H₂₈F₃N₃O₄ (563.58); mass spectrum: (M−H)⁻562; (M+H)⁺=564.

### EXAMPLE 73

N-methyl-N-benzyl-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, N-methylbenzylamine, TBTU and triethylamine in tetrahydrofuran. Yield: 79% of theory; R_{f} value: 0.77 (silica gel; dichloromethane/ethanol=9:1); C₂₈H₂₄F₃N₃O₂ (491.52); mass spectrum: (M−H)⁻=490; (M+H)⁺=492.

### EXAMPLE 74

N-(2-difluoromethoxyphenylmethyl)-4-(4′-trifluoromethylbiphenyl-2-carbonylamino-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 2-difluoromethoxybenzylamine, TBTU and triethylamine in tetrahydrofuran. Yield: 69% of theory; R_{f} value: 0.75 (silica gel; dichloromethane/ethanol=9:1); C₂₈H₂₂F₅N₃O₃ (543.49); mass spectrum: (M−H)⁻=542; (M+H)⁺=544; (M+Na)⁺=566.

### EXAMPLE 75

N-(2-methylphenylmethyl)-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 2-methylbenzylamine, TBTU and triethylamine in tetrahydrofuran. Yield: 66% of theory; R_{f} value: 0.76 (silica gel; dichloromethane/ethanol=9:1); C₂₈H₂₄F₃N₃O₂ (491.52); mass spectrum: (M−H)⁻=490; (M+H)⁺=492.

### EXAMPLE 76

N-[2-(biphenyl-4-yl)ethyl]-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 2-(biphenyl-4-yl)ethylamine, TBTU and triethylamine in tetrahydrofuran. Yield: 88% of theory; R_{f} value: 0.76 (silica gel; dichloromethane/ethanol=9:1); C₃₄H₂₈F₃N₃O₂ (567.61); mass spectrum: (M−H)⁻=566; (M+H)⁺=568; (M+Na)⁺=590.

### EXAMPLE 77

N-[4-(4-methylpiperidino)phenylmethyl)-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 4-(4-methylpiperidino)benzylamine, TBTU and triethylamine in tetrahydrofuran. Yield: 48% of theory; R_{f} value: 0.25 (silica gel; petroleum ether/ethyl acetate=3:2); C₃₃H₃₃F₃N₄O₂ (574.65); mass spectrum: (M−H)⁻=573; (M+H)⁺=575.

### EXAMPLE 78

N-[4-(1.4-dioxa-8-azaspiro[4.5]dec-8-yl)phenylmethyl]-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 4-(1,4-dioxa-8-azaspiro[4.5]dec-8-yl)benzylamine, TBTU and triethylamine in tetrahydrofuran. Yield: 90% of theory; R_{f} value: 0.65 (silica gel; petroleum ether/ethyl acetate=3:2); C₃₄H₃₃F₃N₄O₄ (618.66); mass spectrum: (M−H)⁻=617; (M+H)⁺=619.

### EXAMPLE 79

N-[4-(3-azaspiro[5.5]undec-3-yl)phenylmethyl]-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 4-(3-azaspiro[5.5]undec-3-yl)benzylamine, TBTU and triethylamine in tetrahydrofuran. Yield: 65% of theory; R_{f} value: 0.21 (silica gel; petroleum ether/ethyl acetate=3:2); C₃₇H₃₉F₃N₄O₂ (628.74); mass spectrum: (M+H)⁺=629.

### EXAMPLE 80

N-[1-(4-chlorophenyl)ethyl]-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 1-(4-chlorophenyl)ethylamine, TBTU and triethylamine in tetrahydrofuran. Yield: 100% of theory; R_{f} value: 0.82 (silica gel; dichloromethane/ethanol=9:1); C₂₈H₂₃ClF₃N₃O₂ (525.96); mass spectrum: (M−H)⁻=524/26 (chlorine isotope); (M+H)⁺=526/28 (chlorine isotope).

### EXAMPLE 81

N-[4-(3-methyl-[1.24]oxadiazol-5-yl)methylphenylmethyl]-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 4-(3-methyl-[1,2,4]oxadiazol-5-yl)methylbenzylamine, TBTU and triethylamine in tetrahydrofuran. Yield: 84% of theory; R_{f} value: 0.70 (silica gel; dichloromethane/ethanol=9:1); C₃₁H₂₆F₃N₅O₃ (573.58); mass spectrum: (M−H)⁻=572; (M+H)⁺=574; (M+Na)⁺=596.

### EXAMPLE 82

N-(4-methoxycarbonylcyclohexylmethyl)-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, methyl 4-aminomethylcyclohexanecarboxylate, TBTU and triethylamine in tetrahydrofuran. Yield: 62% of theory; R_{f} value: 0.72 (silica gel; dichloromethane/ethanol=9:1); C₂₉H₃₀F₃N₃O₄ (541.57); mass spectrum: (M−H)⁻=540; (M+H)⁺=542.

### EXAMPLE 83

N-(4-benzyloxybenzyl)-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 4-benzyloxybenzylamine, TBTU and triethylamine in tetrahydrofuran. Yield: 83% of theory; R_{f} value: 0.73 (silica gel; dichloromethane/ethanol=9:1); C₃₄H₂₈F₃N₃O₃ (583.61); mass spectrum: (M+H)⁺=584; (M+Na)⁺=606; (M−H)⁻582; (M+HCOO)⁻=628.

### EXAMPLE 84

N-[4-(3-methylpiperidino)phenylmethyl)-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 4-(3-methylpiperidino)benzylamine, TBTU and triethylamine in tetrahydrofuran. Yield: 16% of theory; R_{f} value: 0.81 (silica gel; dichloromethane/ethanol=9:1); C₃₃H₃₃F₃N₄O₂ (574.65); mass spectrum: (M+H)⁺=575; (M+HCOO)⁻=619.

### EXAMPLE 85I

N-[cyclopropyl-(4-methoxyphenyl)methyl]-4-(4′-trifluoromethylbiphenyl-2-carbonylamino)-1-methylpyrrole-2-carboxylic Acid Amide and N-[1-(4-methoxyphenyl)butyl]-4-(4′-trifluoromethylbiphenyl-2-carbonylamino)-1-methylpyrrole-2-carboxylic Acid Amide in the Ratio 1:1

Prepared analogously to Example 1d from 4-(4′-trifluoromethylbiphenyl-2-carbonylamino)-1-methylpyrrole-2-carboxylic acid, a 1:1 mixture of 1-(4-methoxyphenyl)butylamine and C-cyclopropyl-C-(4-methoxyphenyl)methylamine, TBTU and triethylamine in tetrahydrofuran.

Yield: 100% of theory; R_{f} value: 0.74 (silica gel; dichloromethane/ethanol=9:1). N-[cyclopropyl-(4-methoxyphenyl)methyl]-4-(4′-trifluoromethylbiphenyl-2-carbonylamino)-1-methylpyrrole-2-carboxylic acid amide C₃₁H₂₈F₃N₃O₃ (547.58); mass spectrum: (M)+=547; (M+H)⁺=548; (M+Na)⁺=570; (M−H)⁻546. N-[1-(4-methoxyphenyl)butyl]-4-(4′-trifluoromethylbiphenyl-2-carbonylamino)-1-methylpyrrole-2-carboxylic acid amide C₃₁H₃₀F₃N₃O₃ (549.59); mass spectrum: (M)⁺=549; (M+H)⁺=550; (M+Na)⁺=572; (M−H)⁻548.

### EXAMPLE 86

N-[5-(4-cyano-4-phenylpiperidinocarbonyl)-1-methylpyrrol-3-yl]-4′-trifluoromethylbiphenyl-2-carboxylic Acid Amide

Prepared analogously to Example 1d from 4-(4′-trifluoromethylbiphenyl-2-carbonylamino)-1-methylpyrrole-2-carboxylic acid, 4-cyano-4-phenylpiperidine, TBTU and triethylamine in tetrahydrofuran. Yield: 67% of theory; R_{f} value: 0.83 (silica gel; dichloromethane/ethanol 9:1); C₃₂H₂₇F₃N₄O₂ (556.59); mass spectrum: (M−H)⁻=555; (M+H)⁺=557.

### EXAMPLE 87

N-[4-(9-ethylaminocarbonylfluoren-9-ylbuyl]-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 4-(9-ethylaminocarbonylfluoren-9-yl)butylamine, TBTU and N-ethyldiisopropylamine in dimethylformamide. Yield: quantitative; R_{f} value: 0.60 (silica gel; dichloromethane/ethanol=9:1); C₄₀H₃₇F₃N₄O₃ (678.76); mass spectrum: (M−H)⁻677; (M+Na)⁺=701.

### EXAMPLE 88

N-(4′-methylbiphenyl-4-yl)methyl-4-(4′-trifluoromethylbiphenyl-2-carbonylamino) 1-(3-aminopropyl)pyrrole-2-carboxylic Acid Amide

Prepared analogously to Example 19c from N-(4′-methylbiphenyl-4-yl)methyl-4-(4′-trifluoromethylbiphenyl-2-carbonylamino)-1-(3-tert-butoxycarbonylaminopropyl)pyrrole-2-carboxylic acid amide and trifluoroacetic acid in dichloromethane. Yield: quantitative; R_{f} value: 0.35 (silica gel; dichloromethane/ethanol/ammonia=50:45:5); C₃₆H₃₃F₃N₄O₂ (610.68); mass spectrum: (M−H)⁻=609; (M+H)⁺=611.

### EXAMPLE 89

N-[4-(5-dimethylaminopyridin-2-yl)phenylmethyl]-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 4-(5-dimethylaminopyridin-2-yl)benzylamine, TBTU and N-ethyl-diisopropylamine in dimethylformamide. Yield: 57% of theory; R_{f} value: 0.55 (silica gel; dichloromethane/ethanol=19:1); C₃₄H₃₀F₃N₅O₂ (597.64); mass spectrum: (M−H)⁻596; (M+H)⁺=598; (M+Na)⁺=620.

### EXAMPLE 90

N-[3-(biphenyl-4-ylprop-2-ynyl]-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 3-(biphenyl-4-yl)prop-2-ynylamine-trifluoroacetate, TBTU and N-ethyldiisopropylamine in dimethylformamide. Yield: 22% of theory; R_{f} value: 0.70 (silica gel; dichloromethane/ethanol=9:1); C₃₅H₂₆F₃N₃O₂ (577.60); mass spectrum: (M−H)⁻576; (M+H)⁺=578.

### EXAMPLE 91

N-[3-(4-isopropylphenylprop-2-ynyl]-4-(4′-trifluoromethylbiphenyl-2-carbonylamino)-1-methylimidazole-2-carboxylic Acid Amide

Prepared analogously to Example 11 from 3-(4-isopropylphenyl)prop-2-ynylamine and 4-(4′-trfluoromethylbiphenyl-2-carbonylamino)-1-methylimidazole-2-carboxylic acid in dichloromethane with the addition of propanephosphonic acid cycloanhydride and N-methylmorpholine. Yield: 24% of theory; R_{f} value: 0.49 (silica gel; dichloromethane/ethanol=9:1); C₃ IH₂₇F₃N₄O₂ (544.58); mass spectrum: (M−H)⁻543; (M+Na)⁺=567.

### EXAMPLE 92

N-(4′-methylbiphenyl-4-yl)methyl-4-[2-(pyrrolidin-1-yl)phenylcarbonylamino]-1-methylpyrrole-2-carboxylic Acid Amide

Prepared analogously to Example 1d from 4-[2-(pyrrolidin-1-yl)phenylcarbonylamino)-1-methylpyrrole-2-carboxylic acid, 4′-methylbiphenyl-4-methylamine, TBTU and N-ethyldiisopropylamine in dimethylformamide. Yield: 82% of theory; R_{f} value: 0.60 (silica gel; dichloromethane/ethanol=9:1); C₃₁H₃₂N₄O₂ (492.62); mass spectrum: (M−H)⁻491; (M+Na)⁺=515.

### EXAMPLE 93

N-[5-(1,2,3,4-tetrahydroisoquinolin-2-ylcarbonyl)-1-methylpyrrol-3-yl]-4′-trifluoromethylbiphenyl-2-carboxylic Acid Amide

Prepared analogously to Example 1d from 4-(4′-trifluoromethylbiphenyl-2-carbonylamino)-1-methylpyrrole-2-carboxylic acid, 1,2,3,4-tetrahydroisoquinoline, TBTU and N-ethyldiisopropylamine in dimethylformamide. Yield: 70% of theory; R_{f} value: 0.72 (silica gel; dichloromethane/ethanol=9:1); C₂₉H₂₄F₃N₃O₂ (503.52); mass spectrum: (M−H)⁻=502; (M+H)⁺=504.

### EXAMPLE 94

N-[5-(1,3-dihydroisoindol-2-ylcarbonyl)-1-methylpyrrol-3-yl]-4′-trifluoromethylbiphenyl-2-carboxylic Acid Amide

Prepared analogously to Example 1d from 4-(4′-trifluoromethylbiphenyl-2-carbonylamino)-1-methylpyrrole-2-carboxylic acid, 2,3-dihydro-1H-isoindole, TBTU and N-ethyldiisopropylamine in dimethylformamide. Yield: 79% of theory; R_{f} value: 0.64 (silica gel; dichloromethane/ethanol=9:1); C₂₈H₂₂F₃N₃O₂ (489.50); mass spectrum: (M−H)⁻488; (M+H)⁺=490; (M+Na)⁺=512.

### EXAMPLE 95

N-(4′-methylbiphenyl-4-yl)methyl-4-[1-oxo-7-(4-trifluoromethylphenyl)-1.3-dihydroisoindol-2-yl]-1-methylpyrrole-2-carboxylic Acid Amide

a. methyl 3-methyl-4′-trifluoromethylbiphenyl-2-carboxylate

A mixture of 1.1 g (4.58 mmol) of methyl 2-bromo-6-methylbenzoate, 0.9 g (4.7 mmol) of 4-(trifluoromethyl)benzeneboric acid, 0.3 g (0.26 mmol) of tetrakis(triphenylphosphine)palladium(0) and 0.2 g (0.24 mmol) of 2,2′-bis-(diphenylphosphino)-1,1′-binaphthyl are placed in 150 mL of dimethoxyethane, after 10 minutes combined with 7 mL (7 mmol) of 1 molar sodium carbonate solution and then refluxed for 5 hours. The solvent is distilled off, the residue is distributed in water/dichloromethane, the combined organic extracts are dried and chromatographed on silica gel, eluting with ethyl acetate/petroleum ether 95:5. Yield: 1.1 g (83% of theory); R_{f} value: 0.8 (silica gel; dichloromethane/ethanol=99:1); C₁₆H₁₃F₃O₂ (294.28); mass spectrum: (M+Na)⁺=317.

b. methyl 3-bromomethyl-4′-trifluoromethylbiphenyl-2-carboxylate

0.5 g (1.7 mmol) of methyl 3-methyl-4′-trifluoromethylbiphenyl-2-carboxylate are dissolved in 10 mL of carbon tetrachloride and after the addition of 0.45 g (2.57 mmol) of N-bromosuccinimide and 10 mg (0.06 mmol) of 2,2-azoisobutyronitrile refluxed for 7 hours. The succinimide precipitated is suction filtered and the filtrate is concentrated by evaporation. The residue is chromatographed on silica gel, eluting with petroleum ether/dichloromethane 8:2. Yield: 0.4 g (62% of theory); R_{f} value: 0.45 (silica gel; petroleum ether/ethyl acetate=9:1); C₁₆H₁₂BrF₃O₂ (373.17); mass spectrum: M⁺=372/74 (bromine isotope).

c. methyl 4-[1-oxo-7-(4-trifluoromethylphenyl)-1,3-dihydroisoindol-2-yl]-1-methylpyrrole-2-carboxylate

0.4 g (1.0 mmol) of methyl 3-bromomethyl-4′-trifluoromethylbiphenyl-2-carboxylate are dissolved in 15 mL acetonitrile and after the addition of 0.2 g (1.0 mmol) of methyl 4-amino-1-methylpyrrole-2-carboxylate stirred for 3.5 hours at 80° C. The solvent is distilled off and the residue is chromatographed on silica gel, eluting with petroleum ether/ethyl acetate 85:15 and 75:25. Yield: 0.2 g (43% of theory); R_{f} value: 0.25 (silica gel; dichloromethane/ethanol=99:1); C₂₂H₁₇F₃N₂O₃ (414.39); mass spectrum: (M−H)⁻=413; (M+H)⁺=415; (M+Na)⁺=437.

d. 4-[1-oxo-7-(4-trifluoromethylphenyl)-1.3-dihydroisoindol-2-yl]-1-methylpyrrole-2-carboxylic Acid

Prepared analogously to Example 1e from methyl 4-[1-oxo-7-(4-trifluoromethylphenyl)-1,3-dihydroisoindol-2-yl]-1-methylpyrrole-2-carboxylate and sodium hydroxide solution in methanol. Yield: 85% of theory; R_{f} value: 0.35 (silica gel; dichloromethane/ethanol=19:1); C₂₁H₁₅F₃N₂O₃ (400.36); mass spectrum: (M−H)⁻=399; (M+H)⁺=401; (M+Na)⁺=423.

e. N-(4′-methylbiphenyl-4-yl)methyl-4-[1-oxo-7-(4-trifluoromethylphenyl)-1.3-dihydroisoindol-2-yl]-1-methylpyrrole-2-carboxylic Acid Amide

Prepared analogously to Example 1d from 4-[1-oxo-7-(4-trifluoromethylphenyl)-1,3-dihydroisoindol-2-yl]-1-methylpyrrole-2-carboxylic acid, C-(4′-methylbiphenyl-4-yl)methylamine, TBTU and N-ethyl-diisopropylamine in dimethylformamide. Yield: 96% of theory; R_{f} value: 0.80 (silica gel; dichloromethane/ethanol=9:1); C₃₅H₂₈F₃N₃O₂ (579.62); mass spectrum: (M+H)⁺=580; (M+Na)⁺=602.

### EXAMPLE 96

N-(4-dimethylaminobutyl)-4-(4′-trifluoromethylbiphenyl-2-carbonylamino)-1-methylpyrrole-2-carboxylic Acid Amide

Prepared analogously to Example 1d from 1-amino-4-(dimethylamino)butane, 4-(4′-trifluoromethylbiphenyl-2-carbonylamino)-1-methylpyrrole-2-carboxylic acid, TBTU and triethylamine in dimethylformamide. Yield: 99% of theory; R_{f} value: 0.17 (silica gel; ethyl acetate/ethanol/ammonia=50:45:5); C₂₆H₂₉F₃N₄O₂ (486.54); mass spectrum: (M−H)⁻485; (M+H)⁺=487.

### EXAMPLE 97

N-(4′-methylbiphenyl-4-yl)methyl-4-(4′-trifluoromethylbiphenyl-2-carbonylamino)-1-(2-methoxycarbonylethyl)pyrrole-2-carboxylic Acid Amide

Prepared analogously to Example 4a from 4′-trifluoromethylbiphenyl-2-carboxylic acid chloride, N-(4′-methylbiphenyl-4-yl)methyl-4-amino-1-(2-methoxycarbonylethyl)pyrrole-2-carboxylic acid and triethylamine in tetrahydrofuran. Yield: 80% of theory; R_{f} value: 0.60 (silica gel; dichloromethane/ethanol=9:1); C₃₇H₃₂F₃N₃O₄ (639.68); mass spectrum: (M+H)⁺=640.

### EXAMPLE 98

N-(4-hydroxycarbonylcyclohexylmethyl)-4-(4′-trifluoromethylbiphenyl-2-carbonylamino)-1-methylpyrrole-2-carboxylic Acid Amide

Prepared analogously to Example 1a from N-(4-methoxycarbonylcyclohexylmethyl)-4-(4′-trifluoromethylbiphenyl-2-carbonylamino)-1-methylpyrrole-2-carboxylic acid amide and sodium hydroxide solution in methanol. Yield: 88% of theory; R_{f} value: 0.91 (silica gel; dichloromethane/ethanol=9:1); C₂₈H₂₈F₃N₃O₄ (527.54); mass spectrum: (M−H)⁻526; (M+H)⁺=528.

### EXAMPLE 99

N-(4′-methylbiphenyl-4-yl)methyl-4-(4′-trifluoromethylbiphenyl-2-carbonylamino)-1-(2-hydroxycarbonylethyl)pyrrole-2-carboxylic Acid Amide

Prepared analogously to Example 1e from N-(4′-methylbiphenyl-4-yl)methyl-4-(4′-trifluoromethylbiphenyl-2-carbonylamino)-1-(2-methoxycarbonylethyl)pyrrole-2-carboxylic acid amide and sodium hydroxide solution in methanol. Yield: 62% of theory; R_{f} value: 0.30 (silica gel; dichloromethane/ethanol=9:1); C₃₆H₃₀F₃N₃O₄ (625.65); mass spectrum: (M−H)⁻=624; (M+H)⁺=626; (M+Na)⁺=648.

### EXAMPLE 100

1-methyl-2-[4-(piperidin-1-yl)methylpiperidinocarbonyl]-4-(4′-trifluoromethylbiphenyl-2-carbonylamino)pyrrole

Prepared analogously to Example 1d from 4-(piperidin-1-yl)methylpiperidine, 4-(4′-trifluoromethylbiphenyl-2-carbonylamino)-1-methylpyrrole-2-carboxylic acid, TBTU and triethylamine in dimethylformamide. Yield: 96% of theory; R_{f} value: 0.29 (silica gel; dichloromethane/ethanol=4:1); C₃₁H₃₅F₃N₄O₂ (552.64); mass spectrum: (M−H)⁻551; (M+H)⁺=553.

### EXAMPLE 101

2-[4-(N-acetyl-N-methylaminomethyl)piperidinocarbonyl]-1-methyl-4-(4′-trifluoromethylbiphenyl-2-carbonylamino)pyrrole

Prepared analogously to Example 1d from N-methyl-N-(piperidin-4-yl)methylacetamide, 4-(4′-trifluoromethylbiphenyl-2-carbonylamino)-1-methylpyrrole-2-carboxylic acid, TBTU and triethylamine in dimethylformamide. Yield: quantitative; C₂₉H₃₁F₃N₄O₃ (540.59); mass spectrum: (M−H)⁻=539; (M+H)⁺=541.

### EXAMPLE 102

2-[7-(4-cyanophenoxy)-1,2,3,4-tetrahydroisoquinolin-2-ylcarbonyl]-1-methyl-4-(4′-trifluoromethylbiphenyl-2-carbonylamino)pyrrole

Prepared analogously to Example 1d from 7-(4-cyanophenoxy)-1,2,3,4-tetrahydroisoquinoline, 4-(4′-trifluoromethylbiphenyl-2-carbonylamino)-1-methylpyrrole-2-carboxylic acid, TBTU and triethylamine in dimethylformamide. Yield: 96% of theory; R_{f} value: 0.85 (silica gel; dichloromethane/ethanol=9:1); C₃₆H₂₇F₃N₄O₃ (620.63); mass spectrum: (M−H)⁻619; (M+H)⁺=621.

### EXAMPLE 103

N-(4′-methylbiphenyl-4-yl)methyl-4-(4′-trifluoromethylbiphenyl-2-carbonylamino)-1-isopropylpyrrole-2-carboxylic Acid Amide

a. ethyl 1-isopropyl-4-nitropyrrole-2-carboxylate

0.5 g (2.7 mmol) of ethyl 4-nitropyrrole-2-carboxylate are dissolved in 8 mL of dimethylformamide and after batchwise addition of 73 mg (3 mmol) of sodium hydride stirred for another 45 minutes. Then 0.29 mL (2.9 mmol) of isopropyl iodide are added and the mixture is stirred for 12 hours. The reaction mixture is diluted with water and extracted with dichloromethane. The combined organic extracts are dried and concentrated by evaporation. The residue is chromatographed on silica gel, eluting with dichloromethane. Yield: 0.32 g (49% of theory); R_{f} value: 0.7 (silica gel; dichloromethane/ethanol=99:1).

b. ethyl 4-amino-1-isopropylpyrrole-2-carboxylate

0.32 g (1.4 mmol) of ethyl 1-isopropyl-4-nitropyrrole-2-carboxylate are dissolved in 30 mL of ethanol and, after the addition of 0.15 g of 10% palladium on activated charcoal, hydrogenated with hydrogen at ambient temperature. The catalyst is filtered off and the solution is concentrated by evaporation. Yield: 0.26 g (94% of theory); R_{f} value: 0.15 (silica gel; dichloromethane/ethanol=99:1).

c. ethyl 4-(4′-trifluoromethylbiphenyl-2-carbonylamino)-1-isopropylpyrrole-2-carboxylate

Prepared analogously to Example 4a from 4′-trifluoromethylbiphenyl-2-carboxylic acid chloride, ethyl 4-amino-1-isopropylpyrrole-2-carboxylate and triethylamine in tetrahydrofuran. Yield: 65% of theory; R_{f} value: 0.75 (silica gel; dichloromethane/ethanol=19:1).

d. 4-(4′-trifluoromethylbiphenyl-2-carbonylamino)-1-isopropylpyrrole-2-carboxylic Acid

Prepared analogously to Example 1e from ethyl 4-(4′-trifluoromethylbiphenyl-2-carbonylamino)-1-isopropylpyrrole-2-carboxylate and sodium hydroxide solution in methanol. Yield: 80% of theory; R_{f} value: 0.4 (silica gel; dichloromethane/ethanol=19:1)

e. N-(4′-methylbiphenyl-4-yl methyl-4-(4′-trifluoromethylbiphenyl-2-carbonylamino)-1-isopropylpyrrole-2-carboxylic Acid Amide

Prepared analogously to Example 1d from (4′-methylbiphenyl-4-yl)methylamine, 4-(4′-trifluoromethylbiphenyl-2-carbonylamino)-1-isopropylpyrrole-2-carboxylic acid, TBTU and N-ethyl-diisopropylamine in dimethylformamide. Yield: 94% of theory; R_{f} value: 0.75 (silica gel; dichloromethane/ethanol 9:1); C₃₆H₃₂F₃N₃O₂ (595.67); mass spectrum: (M−H)⁻=594; (M+H)⁺=596.

### EXAMPLE 104

N-[3-(biphenyl-4-yl)propyl]-4-(4′-trifluoromethylbiphenyl-2-carbonylamino)-1-methylimidazole-2-carboxylic Acid Amide

Prepared analogously to Example 103b from N-[3-(4-biphenyl)prop-2-ynyl]-4-(4′-trifluoromethylbiphenyl-2-carbonylamino)-1-methylimidazole-2-carboxylic acid amide and 10% palladium on activated charcoal in ethanol. Yield: 99% of theory; R_{f} value: 0.5 (silica gel; petroleum ether/ethyl acetate=1:1); C₃₄H₂₉F₃N₄O₂ (582.63); mass spectrum: (M−H)⁻581; (M+H)⁺=583.

### EXAMPLE 105

N-(cyclohexylmethyl)-4-(4′-trifluoromethylbiphenyl-2-carbonylamino)-1-methylpyrrole-2-carboxylic Acid Amide

Prepared analogously to Example 1d from (aminomethyl)cyclohexane, 4-(4′-trifluoromethylbiphenyl-2-carbonylamino)-1-methylpyrrole-2-carboxylic acid, TBTU and triethylamine in dimethylformamide. Yield: 99% of theory; R_{f} value: 0.7 (silica gel; dichloromethane/ethanol=9:1); C₂₇H₂₈F₃N₃O₂ (483.53); mass spectrum: (M−H)⁻482; (M+H)⁺=484.

### EXAMPLE 106

N-(4′-methylbiphenyl-4-yl)methyl-4-(2-phenoxyphenylcarbonylamino)-1-methylpyrrole-2-carboxylic Acid Amide

Prepared analogously to Example 1d from 2-phenoxybenzoic acid, N-(4′-methylbiphenyl-4-yl)methyl-4-amino-1-methylpyrrole-2-carboxylic acid amide, TBTU and N-ethyl-diisopropylamine in dimethylformamide. Yield: quantitative; R_{f} value: 0.4 (silica gel; dichloromethane/ethanol=19:1); C₃₃H₂₉N₃O₃ (515.61); mass spectrum: (M+H)⁺=516; (M+HCOO)⁺=560.

### EXAMPLE 107

N-(4′-methylbiphenyl-4-yl)methyl-4-[2-(2-phenylethyl)phenylcarbonylamino]-1-methylpyrrole-2-carboxylic Acid Amide

Prepared analogously to Example 1d from 2-(2-phenylethyl)benzoic acid, N-(4′-methylbiphenyl-4-yl)methyl-4-amino-1-methylpyrrole-2-carboxylic acid amide, TBTU and N-ethyl-diisopropylamine in dimethylformamide. Yield: quantitative; R_{f} value: 0.5 (silica gel; dichloromethane/ethanol=19:1); C₃₅H₃₃N₃O₂ (527.67); mass spectrum: (M−H)⁻=526; (M+H)⁺=528.

### EXAMPLE 108

N-[4-(tert-butoxycarbonylaminomethyl)phenylmethyl]-4-(4′-trifluoromethylbiphenyl-2-carbonylamino)-1-methylpyrrole-2-carboxylic Acid Amide

Prepared analogously to Example 1d from 4-tert-butoxycarbonylaminomethylbenzylamine, 4-(4′-trifluoromethylbiphenyl-2-carbonylamino)-1-methylpyrrole-2-carboxylic acid, TBTU and triethylamine in dimethylformamide. Yield: 96% of theory; R_{f} value: 0.67 (silica gel; dichloromethane/ethanol=9:1); C₃₃H₃₃F₃N₄O₄ (606.65); mass spectrum: (M−H)⁻=605; (M+Na)⁺=629.

### EXAMPLE 109

N-(4-aminomethyl)phenylmethyl-4-(4′-trifluoromethylbiphenyl-2-carbonylamino)-1-methylpyrrole-2-carboxylic Acid Amide

Prepared analogously to Example 19c from N-[4-(tert-butoxycarbonylaminomethyl)phenylmethyl]-4-(4′-trifluoromethylbiphenyl-2-carbonylamino)-1-methylpyrrole-2-carboxylic acid amide and trifluoroacetic acid in dichloromethane. Yield: quantitative; R_{f} value: 0.7 (silica gel; dichloromethane/ethanol=4:1); C₂₈H₂₅F₃N₄O₂ (506.53); mass spectrum: (M−H)⁻=505; (M+H)⁺=507.

### EXAMPLE 110

N-(4′-methylbiphenyl-4-yl)methyl-4-[3-methyl-2-(piperidin-1-yl)phenylcarbonylamino]-1-methylpyrrole-2-carboxylic Acid Amide

Prepared analogously to Example 1d from 3-methyl-2-(piperidin-1-yl)benzoic acid, N-(4′-methylbiphenyl-4-yl)methyl-4-amino-1-methylpyrrole-2-carboxylic acid amide, TBTU and triethylamine in dimethylformamide. Yield: 66% of theory; R_{f} value: 0.4 (silica gel; dichloromethane/ethanol=4:1); C₃₃H₃₆N₄O₂ (520.68); mass spectrum: (M+H)⁺=521.

### EXAMPLE 111

N-(4′-methylbiphenyl-4-ylmethyl-4-[2-(benzylamino)phenylcarbonylamino]-1-methylpvrrole-2-carboxylic Acid Amide

Prepared analogously to Example 1d from N-benzylanthranilic acid, N-(4′-methylbiphenyl-4-yl)methyl-4-amino-1-methylpyrrole-2-carboxylic acid amide, TBTU and triethylamine in dimethylformamide. Yield: 74% of theory; R_{f} value: 0.44 (silica gel; dichloromethane/ethanol=9:1); C₃₄H₃₂N₄O₂ (528.65); mass spectrum: (M−H)⁻527; (M+H)⁺=529.

### EXAMPLE 112

N-(4′-methylbiphenyl-4-ylmethyl-4-[2-(4-methylphenylsulfonylamino)phenylcarbonylamino]-1-methylpyrrole-2-carboxylic Acid Amide

Prepared analogously to Example 1d from 2-(4-methylphenylsulfonylamino)benzoic acid, N-(4′-methylbiphenyl-4-yl)methyl-4-amino-1-methylpyrrole-2-carboxylic acid amide, TBTU and triethylamine in dimethylformamide. Yield: 5% of theory; R_{f} value: 0.65 (silica gel; dichloromethane/ethanol=9:1); C₃₄H₃₂N₄O₄S (592.72); mass spectrum: (M−H)⁻591.

### EXAMPLE 113

N-[4-(4-propylpiperidino)phenylmethyl]-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, 4-(4-propylpiperidino)benzylamine, TBTU and triethylamine in tetrahydrofuran. Yield: 100% of theory; R_{f} value: 0.80 (silica gel; dichloromethane/ethanol=9:1); C₃₅H₃₇F₃N₄O₂ (602.71); mass spectrum: (M+H)⁺=603.

### EXAMPLE 114

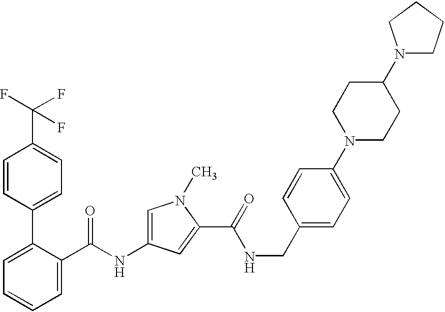

N-{4-[4-(prrolidin-1-yl)piperidino]phenylmethyl}-4-(4′-trifluoromethylbiphenyl-2-carbonylamino)-1-methylpyrrole-2-carboxylic Acid Amide

Can be prepared analogously to Example 1d from 4-(4′-trifluoromethylbiphenyl-2-carbonylamino)-1-methylpyrrole-2-carboxylic acid, 4-[4-(pyrrolidin-1-yl)piperidino]benzylamine, TBTU and triethylamine in tetrahydrofuran.

### EXAMPLE 115

N-[4-(4-phenylpiperidino)phenylmethyl]-4-(4′-trifluoromethylbiphenyl-2-carbonylamino)-1-methylpyrrole-2-carboxylic Acid Amide

Can be prepared analogously to Example 1d from 4-(4′-trifluoromethylbiphenyl-2-carbonylamino)-1-methylpyrrole-2-carboxylic acid, 4-(4-phenylpiperidino)benzylamine, TBTU and triethylamine in tetrahydrofuran.

### EXAMPLE 116

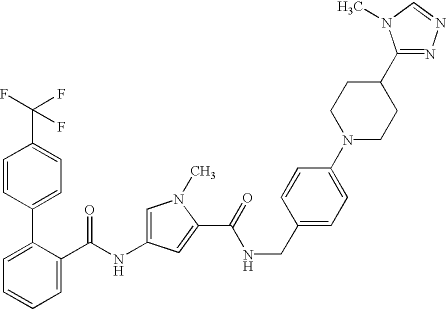

N-{4-[4-(4-methyl-4-H-[1,2,4]triazol-3-yl)piperidino]phenylmethyl}-4-(4′-trifluoromethyl-biphenyl-2-carbonylamino)-1-methylpyrrole-2-carboxylic Acid Amide

Can be prepared analogously to Example 1d from 4-(4′-trifluoromethylbiphenyl-2-carbonylamino)-1-methylpyrrole-2-carboxylic acid, 4-[4-(4-methyl-4-H-[1,2,4]triazol-3-yl)piperidino]benzylamine, TBTU and triethylamine in tetrahydrofuran.

### EXAMPLE 117

N-[4-(4,4-dimethylpiperidinophenylmethyl]-4-(4′-trifluoromethylbiphenyl-2-carbonylamino-1-methylpyrrole-2-carboxylic Acid Amide

Can be prepared analogously to Example 1d from 4-(4′-trifluoromethylbiphenyl-2-carbonylamino)-1-methylpyrrole-2-carboxylic acid, 4-(4,4-dimethylpiperidino)benzylamine, TBTU and triethylamine in tetrahydrofuran.

### EXAMPLE 118

N-{4-[4-(4-methylphenyl)piperidino]phenylmethyl}-4-(4′-trifluoromethylbiphenyl-2-carbonylamino)-1-methylpyrrole-2-carboxylic Acid Amide

Can be prepared analogously to Example 1d from 4-(4′-trifluoromethylbiphenyl-2-carbonylamino)-1-methylpyrrole-2-carboxylic acid, 4-[4-(4-methylphenyl)piperidino]benzylamine, TBTU and triethylamine in tetrahydrofuran.

### EXAMPLE 119

(S)-N-[1-(naphth-2-yl)-ethyl]-4-(4′-trifluoromethylbiphenyl-2-carbonylamino)-1-methylpyrrole-2-carboxylic Acid Amide

Can be prepared analogously to Example 1d from 4-(4′-trifluoromethylbiphenyl-2-carbonylamino)-1-methylpyrrole-2-carboxylic acid, (S)-1-(naphth-2-yl)ethylamine, TBTU and triethylamine in tetrahydrofuran.

### EXAMPLE 120

(R)-N-[1-(naphth-2-yl)ethyl]-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, (R)-1-(naphth-2-yl)ethylamine, TBTU and triethylamine in tetrahydrofuran. Yield: 98% of theory; R_{f} value: 0.79 (silica gel; dichloromethane/ethanol=9:1); C₃₂H₂₆ClF₃N₃O₂ (541.58); mass spectrum: (M−H)⁻540; (M+H)⁺=542; (M+HCOO)⁻=586.

### EXAMPLE 121

### Corresponds to Enantiomerically Pure Example 80

(S)-N-[1-(4-chlorophenyl)ethyl]-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, (R)-1-(4-chlorophenyl)ethylamine, TBTU and triethylamine in tetrahydrofuran. Yield: 77% of theory; R_{f} value: 0.83 (silica gel; dichloromethane/ethanol=9:1); C₂₈H₂₃ClF₃N₃O₂ (525.96); mass spectrum: (M−H)⁻524/26 (chlorine isotope); (M+H)⁺=526/28 (chlorine isotope).

### EXAMPLE 122

### Corresponds to Enantiomerically Pure Example 80

(R)-N-[1-(4-chlorophenyl)-ethyl]-4-(4′-trifluoromethylbiphenyl-2-carbonylamino)-1-methylpyrrole-2-carboxylic Acid Amide

Prepared analogously to Example 1d from 4-(4′-trifluoromethylbiphenyl-2-carbonylamino)-1-methylpyrrole-2-carboxylic acid, (S)-1-(4-chlorophenyl)ethylamine, TBTU and triethylamine in tetrahydrofuran. Yield: 56% of theory; R_{f} value: 0.82 (silica gel; dichloromethane/ethanol=9:1); C₂₈H₂₃ClF₃N₃O₂ (525.96); mass spectrum: (M−H)⁻524/26 (chlorine isotope); (M+H)⁺=526/28 (chlorine isotope).

### EXAMPLE 123

Tablets Containing 5 mg of Active Substance per Tablet

Composition:

| | |
|---|---|
| active substance | 5.0 mg |
| lactose monohydrate | 70.8 mg |
| microcrystalline cellulose | 40.0 mg |
| sodium carboxymethylcellulose, insolubly crosslinked | 3.0 mg |
| magnesium stearate | 1.2 mg |

Preparation:

The active substance is mixed for 15 minutes with lactose monohydrate, microcrystalline cellulose and sodium carboxymethylcellulose in a suitable diffusion mixer. Magnesium stearate is added and mixed with the other substances for another 3 minutes. The finished mixture is compressed in a tablet press to form facetted flat round tablets. Diameter of the tablet: 7 mm; weight of a tablet: 120 mg.

### EXAMPLE 124

Capsules Containing 50 mg of Active Substance per Capsule

Composition:

| | |
|---|---|
| active substance | 50.0 mg |
| lactose monohydrate | 130.0 mg |
| corn starch | 65.0 mg |
| highly dispersed silicon dioxide | 2.5 mg |
| magnesium stearate | 2.5 mg |

Preparation:

A starch paste is prepared by swelling some of the corn starch in a suitable amount of hot water. The paste is then left to cool to room temperature. The active substance is premixed for 15 minutes in a suitable mixer with lactose monohydrate and corn starch. The starch paste is added and the mixture is mixed with sufficient water to produce a moist homogeneous mass. The moist mass is passed through a screen with a mesh size of 1.6 mm. The screened granules are dried on racks at about 55° C. for 12 hours.

The dried granules are then passed through screens with mesh sizes of 1.2 and 0.8 mm. Highly dispersed silica is mixed with the granules in a suitable mixer for 3 minutes. Then magnesium stearate is added and mixing is continued for another 3 minutes. The finished mixture is packed into empty size 1 hard gelatine capsule shells using a capsule filling machine.

### EXAMPLE 125

Tablets Containing 200 mg of Active Substance per Tablet

Composition:

| | |
|---|---|
| active substance | 200.0 mg |
| lactose-monohydrate | 167.0 mg |
| microcrystalline cellulose | 80.0 mg |
| hydroxypropylmethylcellulose, type 2910 | 10.0 mg |
| poly-1-vinyl-2-pyrrolidone, insolubly crosslinked | 20.0 mg |
| magnesium stearate | 3.0 mg |

Preparation:

HPMC is dispersed in hot water. After cooling, the mixture yields a clear solution. The active substance is premixed in a suitable mixer for 5 minutes with lactose monohydrate and microcrystalline cellulose. The HPMC solution is added and the mixing is continued until a homogeneous moist composition is obtained. The moist composition is passed through a screen with a mesh size of 1.6 mm. The screened granules are dried on racks at about 55° C. for 12 hours. The dried granules are then passed through screens with mesh sizes of 1.2 and 0.8 mm. Poly-1-vinyl-2-pyrrolidone is mixed with the granules in a suitable mixer for 3 minutes. Then magnesium stearate is added and mixing is continued for another 3 minutes. The finished mixture is compressed in a tablet press to form oblong tablets (16.2×7.9 mm). Weight of a tablet: 480 mg.

We claim:

1. A compound of formula 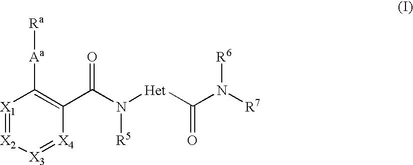
wherein:
X₁ is a nitrogen atom or CR¹;
X₂ is a nitrogen atom or CR²;
X₃ is a nitrogen atom or CR³; and
X₄ is a nitrogen atom or CR⁴,
provided that zero, one, or two of X₁ to X₄ is a nitrogen atom,
wherein R¹, R², R³, and R⁴ are each a hydrogen atom, or one or two of R¹ to R⁴ are each independently a fluorine, chlorine, or bromine atom, or a C₁₋₃-alkyl, a trifluoromethyl, hydroxy, C₁₋₃-alkoxy, trifluoromethoxy, amino, C₁₋₃-alkylamino, or di-(C₁₋₃-alkyl)-amino group and the remainder of R¹ to R⁴ in each case are each a hydrogen atom, and/or
R⁴ together with R⁵ is optionally a —(CH₂)ₙ— bridge wherein n is 1, 2, or 3;
A^{a} is a bond, an oxygen or sulfur atom, or a —NH—, —N(C₁₋₃-alkyl)-, sulfinyl-, sulfonyl-, or carbonyl group, or
—CH₂—, —(CH₂)₂—, —CH═CH—, —C≡C—, —OCH₂—, —CH₂O—, —NH—CH₂—, —CH₂—NH—, —NH—CO—, —CO—NH—, —NH—SO₂—, or —SO₂—NH—, wherein a hydrogen atom bound to a carbon atom and/or a hydrogen atom bound to a nitrogen atom is optionally replaced by a C₁₋₃-alkyl group and wherein a heteroatom of A^{a} is not linked to a nitrogen atom of a 5-membered heteroaryl group of R^{a};
R^{a} is (a) a phenyl, 1-naphthyl, or 2-naphthyl group,
(b) a 5-membered heteroaryl group bound via a carbon or nitrogen atom, which contains:
an imino group optionally substituted by a C₁₋₄-alkyl or C₁₋₄-alkyl-carbonyl group, an oxygen atom, or a sulfur atom,
an imino group optionally substituted by a C₁₋₄-alkyl group, an oxygen atom, or a sulfur atom, and additionally a nitrogen atom, or
an imino group optionally substituted by a C₁₋₄-alkyl group and two nitrogen atoms, or
an oxygen or sulfur atom and two nitrogen atoms,
(c) a 6-membered heteroaryl group which contains one or two nitrogen atoms,
wherein a phenyl ring is optionally fused to the 5- or 6-membered heteroaryl groups of (b) or (c) via two adjacent carbon atoms and the bicyclic heteroaryl groups thus formed is optionally bound via the heteroaromatic or carbocyclic moiety, and
wherein the phenyl and naphthyl groups of (a) as well as the mono- and bicyclic heteroaryl groups of (b) and (c) are optionally monosubstituted in the carbon skeleton by a desired substituent selected from fluorine, chlorine, or bromine atom, or a C₁₋₄-alkyl, C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)amino, acetylamino, N-(C₁₋₃-alkyl)-acetylamino, propionylamino, N-(C₁₋₃-alkyl)-propionylamino, acetyl, propionyl, C₁₋₃-alkoxy-carbonyl, aminocarbonyl, C₁₋₃-alkylaminocarbonyl, di-(C₁₋₃-alkyl)aminocarbonyl, or cyano group or, with the exception of 5-membered heteroaryl groups containing more than two heteroatoms, are also optionally disubstituted by the desired identical or different substituents,
(d) a C₃₋₇-cycloalkyl group, wherein in each case the methylene group in the 4 position of a 6- or 7-membered cycloalkyl group is optionally replaced by an oxygen or sulfur atom, by a sulfinyl or sulfonyl group or by an imino group optionally substituted by a C₁₋₅-alkyl, phenyl, C₁₋₄-alkyl-carbonyl, C₁₋₄-alkoxy-carbonyl, C₁₋₃-alkylaminocarbonyl, or di-(C₁₋₃-alkyl)-aminocarbonyl group,
(e) a 4- to 7-membered cycloalkyleneimino group wherein:
the cycloalkylene moiety is optionally fused to a phenyl ring, or
one or two hydrogen atoms are optionally replaced by a C₁₋₃-alkyl group, and/or
if the 4- to 7-membered cycloalkyleneimino group is a 6- or 7-membered cycloalkyleneimino group, the methylene group in the 4 position of the 6- or 7-membered cycloalkyleneimino group is optionally substituted by a hydroxycarbonyl, C₁₋₃-alkoxy-carbonyl, amino, C₁₋₃-alkylamino, di-(C₁₋₃-alkyl)amino, aminocarbonyl, C₁₋₃-alkylaminocarbonyl, di-(C₁₋₃-alkyl)-aminocarbonyl, or phenyl-C₁₋₃-alkylamino group, or is optionally replaced by an oxygen or sulfur atom, by a sulfinyl or sulfonyl group, or by an imino group optionally substituted by a C₁₋₅-alkyl, phenyl, C₁₋₄-alkyl-carbonyl, C₁₋₄-alkoxy-carbonyl, C₁₋₃-alkylaminocarbonyl, or di-(C₁₋₃-alkyl)-aminocarbonyl group, or
if the 4- to 7-membered cycloalkyleneimino group is a 5-, 6-, or 7-membered cycloalkyleneimino group, a —CH₂— group linked to the imino nitrogen atom is optionally replaced by a carbonyl group, or a —(CH₂)₂— group linked to the imino nitrogen atom is optionally replaced by a —CO—NR⁸— group, or a —(CH₂)₃— group linked to the imino nitrogen atom is optionally replaced by a —CO—NR⁸—CO— group;
R⁵ is a hydrogen atom or a C₁₋₅-alkyl group;
Het is a 5-membered heteroarylene group bound via two carbon atoms or, if Het is a double-bonded pyrrole group, it is optionally also bound via a carbon atom and the imino-nitrogen atom linked to the adjacent carbonyl group in formula I, which contains:
an imino group substituted by R⁹, an oxygen or sulfur atom, or an imino group substituted by R⁹ or an oxygen or sulfur atom and additionally a nitrogen atom, wherein R⁹ is a hydrogen atom; a C₁₋₅-alkyl group; a C₂₋₃-alkyl group terminally substituted by an amino, C₁₋₃-alkylamino, di-(C₁₋₃-alkyl)-amino, or C₁₋₅-alkoxy-carbonylamino group; a carboxy-C₁₋₃-alkyl; C₁₋₃-alkoxy-carbonyl-C₁₋₃-alkyl; phenyl; phenyl-C₁₋₃-alkyl; C₁₋₅-alkyl-carbonyl; or phenylcarbonyl group, or R⁹ together with R⁶ is a —(CH₂)ₚ— bridge, wherein p is 2 or 3, or
an imino group optionally substituted by a C₁₋₃-alkyl group and two nitrogen atoms, or an oxygen or sulfur atom and two nitrogen atoms,
or is a 6-membered heteroarylene group which contains one or two nitrogen atoms,
wherein the heteroarylene group of Het is optionally monosubstituted in the carbon skeleton by a Het substituent selected from: fluorine, chlorine, or bromine atom, a C₁₋₅-alkyl, a C₃₋₇-cycloalkyl, trifluoromethyl, hydroxy, C₁₋₃-alkoxy, trifluoromethoxy, amino, C₁₋₃-alkylamino, di-(C₁₋₃-alkyl)amino, acetylamino, N-(C₁₋₃-alkyl)-acetylamino, propionylamino, N-(C₁₋₃-alkyl)-propionylamino, acetyl, propionyl, benzoyl, C₁₋₃-alkoxy-carbonyl, aminocarbonyl, C₁₋₃-alkylaminocarbonyl-di-(C₁₋₃-alkyl)aminocarbonyl, or cyano group or, with the exception of 5-membered monocyclic heteroaryl groups containing more than one heteroatom, are also optionally disubstituted by identical or different Het substituents;
R⁶ is a hydrogen atom or a C₁₋₆-alkyl group,
R⁷ is (i) a C₁₋₉-alkyl group,
(ii) a straight-chain or branched, mono-, di-, or triunsaturated C₃₋₉-alkenyl or C₃₋₉-alkynyl group, wherein the multiple bonds are isolated from the nitrogen-carbon bond,
(iii) a straight-chain C₂₋₆-alkyl group terminally substituted by an amino, C₁₋₃-alkylamino, or di-(C₁₋₃-alkyl)-amino group,
(iv) a C₁₋₆-alkyl group substituted by a C₃₋₇-cycloalkyl group, wherein:
a hydrogen atom in the 3 position of a cyclopentyl group and in the 4 position of a 6- or 7-membered cycloalkyl group thereof is optionally replaced in each case by a hydroxy, hydroxy-C₁₋₃-alkyl, C₁₋₅-alkoxy, C 1-5-alkoxy-C 1-3-alkyl, phenyl-C₁₋₃-alkoxy-C₁₋₃-alkyl, amino, C₁₋₅-alkylamino, di-(C₁₋₅-alkyl)amino, phenyl-C₁₋₃-alkylamino, C₁₋₅-alkyl-carbonylamino, benzoylamino, amino-C₁₋₃-alkyl, C₁₋₃-alkylamino-C₁₋₃-alkyl, di-(C₁₋₃-alkyl)amino-C₁₋₃-alkyl, phenyl-C₁₋₃-alkylamino-C₁₋₃-alkyl, C₁₋₃-alkyl-carbonylamino-C₁₋₃-alkyl, benzoylamino-C₁₋₃-alkyl, phenylaminocarbonyl, phenyl-C₁₋₃-alkylaminocarbonyl, carboxy, or C₁₋₃-alkoxy-carbonyl group, or
in each case the methylene group in the 4 position of a 6- or 7-membered cycloalkyl group is optionally replaced by an oxygen or sulfur atom, or by an imino group optionally substituted by a C₁₋₆-alkyl, phenyl, C₁₋₆-alkyl-carbonyl, benzoyl, phenyl-(C₁₋₃-alkyl)-carbonyl, C₁₋₆-alkylaminocarbonyl, di-(C₁₋₅-alkyl)-aminocarbonyl, phenylaminocarbonyl, N-(C₁₋₃-alkyl)-phenylaminocarbonyl, phenyl-C₁₋₃-alkylaminocarbonyl, or N-(C₁₋₃-alkyl)-phenyl-C₁₋₃-alkylaminocarbonyl group, or
in a 5- or 6-membered cycloalkyl group, one or two single bonds separated from each other by at least one bond and separated from position 1 are optionally fused to a phenyl group, wherein in a bi- or tricyclic ring system thus formed, the hydrogen atom bound to the saturated carbon atom in position 1 is optionally replaced by a C₁₋₅-alkylaminocarbonyl, di-(C₁₋₅-alkyl)aminocarbonyl, phenyl-C₁₋₃-alkylaminocarbonyl, or C₁₋₅-alkoxy-carbonyl group, wherein the terminal methyl groups in each case are optionally wholly or partially fluorinated,
(v) a C₁₋₆-alkyl group optionally substituted by a C₃₋₇-cycloalkyl group, wherein the C₁₋₆-alkyl group is also optionally substituted
by a carboxy or C₁₋₃-alkoxy-carbonyl group,
by a phenyl, 1-naphthyl, or 2-naphthyl group,
by a 5-membered heteroaryl group bound via a carbon or nitrogen atom, which contains:
an imino group optionally substituted by a C₁₋₃-alkyl, trifluoromethyl, phenyl, phenyl-C₁₋₃-alkyl, C₁₋₃-alkyl-carbonyl, phenylcarbonyl, or phenyl-C₁₋₃-alkyl-carbonyl group, or an oxygen or sulfur atom,
an imino group optionally substituted by a C₁₋₃-alkyl group, or an oxygen or sulfur atom and additionally a nitrogen atom, or
an imino group optionally substituted by a C₁₋₃-alkyl group and two nitrogen atoms, or
an oxygen or sulfur atom and two nitrogen atoms, or
by a 6-membered heteroaryl group, which contains one or two nitrogen atoms,
wherein a phenyl ring is optionally fused to the 5- or 6-membered heteroaryl groups of (v) via two adjacent carbon atoms and the bicyclic heteroaryl groups thus formed is optionally bound via the heteroaromatic or carbocyclic moiety,
wherein the phenyl and naphthyl groups of (v) as well as the mono- and bicyclic heteroaryl groups of (v) are optionally monosubstituted in the carbon skeleton by a fluorine, chlorine, or bromine atom, or by a C₁₋₅-alkyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, amino-C₁₋₃-alkyl, C₁₋₃-alkylamino-C₁₋₃-alkyl, di-(C₁₋₃-alkyl)amino-C₁₋₃-alkyl, C₁₋₅-alkoxy-carbonylamino-C₁₋₃-alkyl, acetylamino, propionylamino, N-(C₁₋₃-alkyl)-benzoylamino, acetyl, propionyl, carboxy, C₁₋₃-alkoxy-carbonyl, C₁₋₃-alkoxy-carbonyl-C₁₋₃-alkyl, aminocarbonyl, C₁₋₃-alkylaminocarbonyl, di-(C₁₋₃-alkyl)aminocarbonyl, or cyano group or, with the exception of 5-membered heteroaryl groups containing more than two heteroatoms, they are optionally disubstituted by identical or different substituents selected from these substituents,
(vi) a C₁₋₆-alkyl group substituted by a phenyl group and a carboxy, C₁₋₃-alkoxy-carbonyl, aminocarbonyl, C₁₋₃-alkylaminocarbonyl, or di-(C₁₋₃-alkyl)-aminocarbonyl group,
(vii) a phenyl-C₂₋₅-alkenylene-CH₂, phenyl-C₂₋₅-alkynylene-CH₂, heteroaryl-C₂₋₅-alkenylene-CH₂, or heteroaryl-C₂₋₅-alkynylene-CH₂ group, wherein a hydrogen atom of the methylene group in position 1 is optionally replaced by a C₁₋₃-alkyl group and independently thereof the phenyl moiety or the heteroaryl moiety thereof is optionally mono- or disubstituted by identical or different substituents selected from fluorine, chlorine, or bromine atoms, or C₁₋₆-alkyl, C₃₋₇-cycloalkyl, trifluoromethyl, C₁₋₃-alkoxy, phenyl, heteroaryl, or cyano groups, wherein disubstitution by two aromatic groups is excluded,
wherein the heteroaryl of (vii) is a 5-membered heteroaryl group bound via a carbon or nitrogen atom, which contains:
an imino group substituted optionally by a C₁₋₃-alkyl group, or an oxygen or sulfur atom,
an imino group substituted optionally by a C₁₋₃-alkyl group, or an oxygen or sulfur atom and additionally a nitrogen atom, or
an imino group substituted optionally by a C₁₋₃-alkyl group and two nitrogen atoms, or
an oxygen or sulfur atom and two nitrogen atoms,
or a 6-membered heteroaryl group, which contains one or two nitrogen atoms,
wherein a phenyl ring is optionally fused to the 5- or 6-membered heteroaryl groups of (vii) via two adjacent carbon atoms and the bicyclic heteroaryl groups thus formed are optionally bound via the heteroaromatic or carbocyclic moiety,
(viii) R^{b}-A^{b}-E^{b}-C₁₋₃-alkyl optionally substituted in the C₁₋₃-alkyl moiety by a C₁₋₄-alkyl or C₃₋₅-cycloalkyl group, wherein:
R^{b} is a phenyl group optionally mono- or disubstituted by fluorine, chlorine, bromine, or iodine atoms, or by C₁₋₄-alkyl, C₂₋₄-alkenyl, C₂₋₄-allynyl, C₃₋₇-cycloalkyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, amino-C₁₋₃-alkyl, C₁₋₃-alkylamino-C₁₋₃-alkyl, di-(C₁₋₃-alkyl)amino-C₁₋₃-alkyl, acetylamino, propionylamino, acetyl, propionyl, carboxy, C₁₋₃-alkoxy-carbonyl, C₁₋₃-alkoxy-carbonyl-C₁₋₃-alkyl, aminocarbonyl, C₁₋₃-alkylaminocarbonyl, di-(C₁₋₃-alkyl)aminocarbonyl, or cyano groups, wherein the substituents are identical or different, or
a 5-membered heteroaryl group which is optionally bound via a carbon atom or, if A^{b} is a bond, a —CH₂, —(CH₂)₂, sulfonyl, or carbonyl group, also is optionally bound via a nitrogen atom and which contains:
an imino group optionally substituted by a C₁₋₃-alkyl group, or an oxygen or sulfur atom,
an imino group optionally substituted by a C₁₋₃-alkyl group, or an oxygen or sulfur atom, and additionally a nitrogen atom,
an imino group optionally substituted by a C₁₋₃-alkyl group and two nitrogen atoms, or
an oxygen or sulfur atom and two nitrogen atoms,
a 6-membered heteroaryl group, which contains one or two nitrogen atoms,
wherein a phenyl ring is optionally fused to the 5- or 6-membered heteroaryl groups of R^{b} via two adjacent carbon atoms and the bicyclic heteroaryl groups thus formed is optionally bound via the heteroaromatic or carbocyclic moiety,
wherein the mono- and bicyclic heteroaryl groups of R^{b} are optionally monosubstituted in the carbon skeleton by a fluorine, chlorine, or bromine atom, or by a C₁₋₄-alkyl, C₂₋₄-alkenyl, C₂₋₄-alkynyl, C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)-amino, acetylamino, propionylamino, acetyl, propionyl, C₁₋₃-alkoxy-carbonyl, aminocarbonyl, C₁₋₃-alkylaminocarbonyl, di-(C₁₋₃-alkyl)aminocarbonyl, or cyano group or, with the exception of 5-membered heteroaryl groups containing more than two heteroatoms, they are optionally disubstituted by identical or different substituents selected from these substituents,
a C₃₋₇-cycloalkyl group, wherein:
one or two hydrogen atoms in each case are optionally replaced by a CI,₃-alkyl group and/or
in each case the methylene group in the 4 position of a 6- or 7-membered cycloalkyl group is optionally replaced by an oxygen or sulfur atom, by a sulfinyl, sulfonyl, or by an imino group optionally substituted by a C₁₋₃-alkyl, C₁₋₃-alkyl-carbonyl, C₁₋₃-alkoxy-carbonyl, C₁₋₃-alkylaminocarbonyl, or di-(C₁₋₃-alkyl)-aminocarbonyl group, or
the two hydrogen atoms of the methylene group in the 3 position of a cyclopentyl group or in 3 or 4 position of a cyclohexyl or cycloheptyl group is optionally replaced by an n-butylene, n-pentylene, n-hexylene, 1,2-ethylenedioxy, or 1,3-propylenedioxy group and in the rings thus formed, one or two hydrogen atoms is optionally replaced by C₁₋₃-alkyl groups,
a 4- to 7-membered cycloalkyleneimino group, wherein:
the cycloalkylene moiety is optionally fused to a phenyl ring, or
one or two hydrogen atoms in each case are optionally replaced by a C₁₋₃-alkyl group and/or
in each case the carbon atom in the 4 position of a 6- or 7-membered cycloalkyleneimino group is optionally substituted by a hydroxy-C₁₋₃-alkyl, C₁₋₆-alkoxy-C₁₋₃-alkyl, hydroxycarbonyl, C₁₋₆-alkoxy-carbonyl, aminocarbonyl, C₁₋₃-alkylaminocarbonyl, di-(C₁₋₃-alkyl)-aminocarbonyl-, 4- to 7-membered cycloalkyleneimino, phenyl, 4-(C₁₋₃-alkyl)-1,2,4-triazol-3-yl, phenyl-C₁₋₃-alkylamino, or N-(C₁₋₃-alkyl)-phenyl-C₁₋₃-alkylamino group, or
is optionally replaced by an oxygen or sulfur atom, by a sulfinyl or sulfonyl group, or by an imino group optionally substituted by a C₁₋₃-alkyl, phenyl, C₁₋₃-alkyl-carbonyl, benzoyl, phenyl-C₁₋₃-alkyl-carbonyl, C₁₋₃-alkylaminocarbonyl, di-(C₁₋₃-alkyl)-aminocarbonyl, phenylaminocarbonyl, or N-(C₁₋₃-alkyl)-phenylaminocarbonyl group, or
the two hydrogen atoms of the methylene group in the 3 position of a 5-membered cycloalkyleneimino group or in the 3 or 4 position of a 6- or 7-membered cycloalkyleneimino group is optionally replaced by an n-butylene, n-pentylene, n-hexylene, 1,2-ethylenedioxy, or 1,3-propylenedioxy group and in the rings thus formed, one or two hydrogen atoms is optionally replaced by C₁₋₃-alkyl groups, or
in a 5-, 6-, or 7-membered cycloalkyleneimino group, a —CH₂— group linked to the imino nitrogen atom is optionally replaced by a carbonyl group, or a —(CH₂)₂— group linked to the imino nitrogen atom is optionally replaced by a —CO—NR⁸— group, or a —(CH₂)₃— group linked to the imino nitrogen atom is optionally replaced by a —CO—NR⁸—CO— group,
A^{b} is a bond, an oxygen or sulfur atom, —NH, —N(C₁₋₃-alkyl), sulfinyl, sulfonyl, or a carbonyl group, or
—CH₂—, —(CH₂)₂—, —O—CH₂—, —CH₂—O—, NH—CH₂—, —CH₂—NH—, —NH—CO—, —CO—NH—, —NH—SO₂—, —SO₂—NH—, —CH═CH—, or —C≡C—, wherein a hydrogen atom bound to a carbon atom and/or a hydrogen atom bound to a nitrogen atom is optionally replaced by a C₁₋₃-alkyl group in each case and a heteroatom of A^{b} is not linked to a nitrogen atom of a 5-membered heteroaryl group of R^{b},
E^{b} is a phenylene group optionally substituted by a fluorine, chlorine, or bromine atom, or by a C₁₋₄-alkyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, amino-C₁₋₃-alkyl, C₁₋₃-alkylamino-C₁₋₃-alkyl, di-(C₁₋₃-alkyl)amino-C₁₋₃-alkyl, acetylamino, propionylamino, acetyl, propionyl, carboxy, C₁₋₃-alkoxy-carbonyl, C₁₋₃-alkoxy-carbonyl-C₁₋₃-alkyl, aminocarbonyl, C₁₋₃-alkylaminocarbonyl, di-(C₁₋₃-alkyl)aminocarbonyl, or cyano group,
(ix) R^{c}-A^{c}-E^{c}-C₁₋₃-alkyl optionally substituted in the C₁₋₃-alkyl moiety by a C₁₋₄-alkyl or C₃-s-cycloalkyl group, wherein:
R^{c} is R^{b}, wherein any reference in R^{b} to A^{b} is replaced by reference to A^{c},
A^{c} is A^{b}, wherein any reference in A^{b} to R^{b} is replaced by reference to R^{c}, and
E^{c} is a 5-membered heteroarylene group bound via two carbon atoms or via a carbon atom and an imino-nitrogen atom, wherein the imino-nitrogen atom of the heteroarylene group is not linked to a heteroatom of A^{c} and the heteroarylene group contains:
an imino group optionally substituted by a C₁₋₃-alkyl group, or an oxygen or sulfur atom,
an imino group optionally substituted by a C₁₋₃-alkyl group, or an oxygen or sulfur atom, and additionally a nitrogen atom, or
an imino group optionally substituted by a C₁₋₃-alkyl group and two nitrogen atoms, or
an oxygen or sulfur atom and two nitrogen atoms,
or a 6-membered heteroarylene group, which contains one or two nitrogen atoms,
wherein a phenyl ring is optionally fused to the 5-membered heteroarylene groups containing one or two heteroatoms as well as to the 6-membered heteroarylene groups via two adjacent carbon atoms and the bicyclic heteroarylene groups thus formed are optionally bound via the heteroaromatic and/or carbocyclic moiety,
and wherein the mono- and bicyclic heteroarylene groups of EC are optionally substituted in the carbon skeleton by a fluorine, chlorine, or bromine atom, or by a C₁₋₄-alkyl, C₃₋₇-cycloalkyl, trifluoromethyl, hydroxy, C₁₋₃-alkoxy, trifluoromethoxy, amino, C₁₋₃-alkylamino, acetylamino, propionylamino, acetyl, propionyl, C₁₋₃-alkoxy-carbonyl, aminocarbonyl, C₁₋₃-alkylaminocarbonyl, or cyano group, or
R⁶ and R⁷ together are an n-alkylene bridge with 3 to 6 carbon atoms, wherein:
one or two hydrogen atoms is optionally replaced by a C₁₋₃-alkyl group, and/or
a —CH₂—CH₂ group is optionally replaced by a 1,2-linked phenylene group optionally mono- or disubstituted by fluorine, chlorine, or bromine atoms, by C₁₋₃-alkyl, trifluoromethyl, hydroxy, C₁₋₃-alkoxy, trifluoromethoxy, amino, C₁₋₃-alkylamino, di-(C₁₋₃-alkyl)amino, acetylamino, propionylamino, acetyl, propionyl, C₁₋₃-alkoxy-carbonyl, aminocarbonyl,
C₁₋₃-alkylaminocarbonyl, cyano, or phenyloxy groups, or monosubstituted by a phenyl-C₁₋₃-alkyl group, wherein the phenyloxy- and phenyl-C₁₋₃-alkyl group in the phenyl moiety are optionally substituted by a fluorine, chlorine, or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino, or cyano group,
or in each case the carbon atom in the 3 position of an n-pentylene or n-hexylene group is optionally monosubstituted by a C₁₋₃-alkyl group terminally substituted by a phenyl, cyano, hydroxy, C₁₋₃-alkoxy, amino, C₁₋₃-alkylamino, di-(C₁₋₃-alkyl)-amino, or a 5- to 7-membered cycloalkyleneimino group, or by a carboxy, C₁₋₃-alkoxy-carbonyl, amino-C₁₋₃-alkyl, C₁₋₃-alkylamino-C₁₋₃-alkyl, N—C₁₋₃-alkyl-N-(C₁₋₃-alkyl-carbonyl)-amino-C₁₋₃-alkyl, di-(C₁₋₃-alkyl)-amino-C₁₋₃-alkyl, aminocarbonyl, C₁₋₃-alkylaminocarbonyl, or di-(C₁₋₃-alkyl)-amino-carbonyl group, or is optionally disubstituted by a phenyl group and a cyano, hydroxy, or C₁₋₃-alkoxy group, or
the methylene group in the 3 position of an n-pentylene or n-hexylene group is optionally replaced by an oxygen or sulfur atom, by a sulfinyl or sulfonyl group, or by an imino group optionally substituted by a C₁₋₃-alkyl, phenyl-C₁₋₃-alkyl, C₁₋₃-alkyl-carbonyl, benzoyl, C₁₋₃-alkylaminocarbonyl, di-(C₁₋₃-alkyl)-aminocarbonyl, phenylaminocarbonyl, or N-(C₁₋₃-alkyl)-phenylaminocarbonyl group, or
a methylene group in position 1 of an n-butylene, n-pentylene, or n-hexylene group, is optionally replaced by a carbonyl group; and
R⁸ is a hydrogen atom or a C₁₋₃-alkyl group,
wherein:
the unsubstituted or monosubstituted phenyl groups and the aromatic or heteroaromatic moieties are, unless otherwise stated, optionally additionally substituted in the carbon skeleton by identical or different substituents selected from fluorine, chlorine, or bromine atoms, C₁₋₃-alkyl groups, trifluoromethyl, hydroxy, C₁₋₃-alkoxy, trifluoromethoxy, amino, C₁₋₃-alkylamino, acetylamino,
acetyl, C₁₋₃-alkoxy-carbonyl, aminocarbonyl, C₁₋₃-alkylaminocarbonyl, or cyano groups, so that the resulting aromatic or heteroaromatic moieties are each optionally at most disubstituted,
the hydrogen atoms in the C₁₋₃-alkyl and alkoxy groups are optionally wholly or partially replaced by fluorine atoms, and
the alkyl and alkoxy groups and the alkyl moieties contained in formula I with more than two carbon atoms are each optionally straight-chain or branched, unless otherwise specified,
the carboxy groups are optionally replaced by a group convertible into a carboxy group in vivo or by a group which is negatively charged under physiological conditions, and
the amino and imino groups are optionally substituted by a group which can be cleaved in vivo,
or a tautomer, enantiomer, diastereomer, or salt thereof.

2. The compound of formula I according to claim 1, wherein:
A^{a} is a bond, an oxygen atom, or a —NH—, —N(C₁₋₃-alkyl)-, sulfonyl-, or carbonyl group, or
a —CH₂—, —(CH₂)₂—, —NH—CH₂—, —CH₂—NH—, —NH—CO—, —CO—NH—, —NH—SO₂—, or —SO₂—NH—, wherein a hydrogen atom bound to a carbon atom and/or a hydrogen atom bound to a nitrogen atom is optionally replaced by a C₁₋₃-alkyl group and wherein a heteroatom of A^{a} is not linked to a nitrogen atom of a 5-membered heteroaryl group of R^{a};
R^{a} is (a) a phenyl group,
(b) a 5-membered heteroaryl group bound via a carbon or nitrogen atom, which contains:
an imino group optionally substituted by a C₁₋₄-alkyl or C₁₋₄-alkyl-carbonyl group, an oxygen atom, or a sulfur atom, or
an imino group optionally substituted by a C₁₋₄-alkyl group, an oxygen atom, or a sulfur atom, and additionally a nitrogen atom,
(c) a 6-membered heteroaryl group which contains one or two nitrogen atoms,
wherein the phenyl group of (a) as well as the heteroaryl groups of (b) and (c) are optionally monosubstituted in the carbon skeleton by a desired substituent selected from fluorine, chlorine, or bromine atom, C₁₋₄-alkyl, C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)amino, acetylamino, N-(C₁₋₃-alkyl)-acetylamino, acetyl, or cyano group,
(d) a C₃₋₇-cycloalkyl group, wherein in each case the methylene group in the 4 position of a 6-membered cycloalkyl group is optionally replaced by an oxygen or sulfur atom, by a sulfonyl group, or by an imino group optionally substituted by a C₁₋₃-alkyl, phenyl, C₁₋₄-alkyl-carbonyl, or C₁₋₄-alkoxy-carbonyl group,
(e) a 4- to 7-membered cycloalkyleneimino group wherein:
one or two hydrogen atoms are optionally replaced by a C₁₋₃-alkyl group, and/or
if the 4- to 7-membered cycloalkyleneimino group is a 6- or 7-membered cycloalkyleneimino group, the methylene group in the 4 position of the 6- or 7-membered cycloalkyleneimino group is optionally replaced by an oxygen or sulfur atom, by a sulfonyl group, or by an imino group optionally substituted by a C₁₋₅-alkyl, phenyl, C₁₋₄-alkyl-carbonyl, C₁₋₄-alkoxy-carbonyl, C₁₋₃-alkylaminocarbonyl, or di-(C₁₋₃-alkyl)-aminocarbonyl group, or
if the 4- to 7-membered cycloalkyleneimino group is a 5-, 6-, or 7-membered cycloalkyleneimino group, a —CH₂— group linked to the imino nitrogen atom is optionally replaced by a carbonyl group, or a —(CH₂)₂— group linked to the imino nitrogen atom is optionally replaced by a —CO—NR⁸— group, or a —(CH₂)₃— group linked to the imino nitrogen atom is optionally replaced by a —CO—NR⁸—CO— group;
R⁵ is a hydrogen atom or a C₁₋₃-alkyl group;
Het is a 5-membered heteroarylene group bound via two carbon atoms, which contains:
an imino group substituted by R⁹, an oxygen or sulfur atom, or an imino group substituted by R⁹ or an oxygen or sulfur atom and additionally a nitrogen atom, wherein R⁹ is a hydrogen atom; a C₁₋₅-alkyl group; a C₂₋₃-alkyl group terminally substituted by an amino, C₁₋₃-alkylamino, di-(C₁₋₃-alkyl)-amino, or C₁₋₅-alkoxy-carbonylamino group; a carboxy-C₁₋₃-alkyl; C₃-alkoxy-carbonyl-C₁₋₃-alkyl; phenyl; phenyl-C₁₋₃-alkyl; C₁₋₅-alkyl-carbonyl; or phenylcarbonyl group, or R⁹ together with R⁶ is a —(CH₂)ₚ— bridge, wherein p is 2 or 3, or
an imino group optionally substituted by a C₁₋₃-alkyl group and two nitrogen atoms, or
an oxygen or sulfur atom and two nitrogen atoms,
or is a 6-membered heteroarylene group which contains one or two nitrogen atoms,
wherein the heteroarylene group of Het is optionally monosubstituted in the carbon skeleton by a Het substituent selected from: fluorine, chlorine, or bromine atom, a C₁₋₃-alkyl, a cyclopropyl, trifluoromethyl, C₁₋₃-alkoxy, trifluoromethoxy, C₁₋₃-alkylamino, di-(C₁₋₃-alkyl)amino, acetylamino, N-(C₁₋₃-alkyl)-acetylamino, acetyl, C₁₋₃-alkylaminocarbonyl-, or di-(C₁₋₃-alkyl)aminocarbonyl group;
R⁶ is a hydrogen atom or a C₁₋₄-alkyl group,
R⁷ is (i) a C₁₋₆-alkyl group,
(ii) a straight-chain C₂₋₆-alkyl group terminally substituted by an amino, C₁₋₃-alkylamino, or di-(C₁₋₃-alkyl)-amino group,
(iii) a C₁₋₆-alkyl group substituted by a C₃₋₇-cycloalkyl group, wherein:
a hydrogen atom in the 3 position of a cyclopentyl group and in the 4 position of a 6- or 7-membered cycloalkyl group thereof is optionally replaced in each case by a C₁₋₅-alkoxy, phenyl-C₁₋₃-alkoxy-C₁₋₃-alkyl, phenyl-C₁₋₃-alkylamino, C₁₋₅-alkyl-carbonylamino, benzoylamino, phenyl-C₁₋₃-alkylamino-C₁₋₃-alkyl, benzoylamino-C₁₋₃-alkyl, phenylaminocarbonyl, phenyl-C₁₋₃-alkylaminocarbonyl, carboxy, or C₁₋₃-alkoxy-carbonyl group, or
in each case the methylene group in the 4 position of a 6- or 7-membered cycloalkyl group is optionally replaced by an imino group optionally substituted by a phenyl, C₁₋₆-alkyl-carbonyl, benzoyl, phenyl-(C₁₋₃-alkyl)-carbonyl, phenylaminocarbonyl, N-(C₁₋₃-alkyl)-phenylaminocarbonyl, phenyl-C₁₋₃-alkylaminocarbonyl, or N-(C₁₋₃-alkyl)-phenyl-C₁₋₃-alkylaminocarbonyl group, or
in a 5- or 6-membered cycloalkyl group, one or two single bonds separated from each other by at least one bond and separated from position 1 are optionally fused to a phenyl group, wherein in a bi- or tricyclic ring system thus formed, the hydrogen atom bound to the saturated carbon atom in position 1 is optionally replaced by a C₁₋₃-alkylaminocarbonyl, di-(C₁₋₃-alkyl)aminocarbonyl, or C₁₋₅-alkoxy-carbonyl group, wherein the terminal methyl groups in each case are optionally wholly or partially fluorinated,
(iv) a C₁₋₆-alkyl group optionally substituted by a C₃₋₇-cycloalkyl group, wherein the C₁₋₆-alkyl group is optionally substituted
by a carboxy or C₁₋₃-alkoxy-carbonyl group,
by a phenyl, 1-naphthyl, or 2-naphthyl group,
by a 5-membered heteroaryl group bound via a carbon or nitrogen atom, which contains:
an imino group optionally substituted by a C₁₋₃-alkyl or trifluoromethyl group, or
an oxygen or sulfur atom, or an imino group optionally substituted by a C₁₋₃-alkyl group, or an oxygen or sulfur atom and additionally a nitrogen atom, or
by a 6-membered heteroaryl group, which contains one or two nitrogen atoms,
wherein the phenyl groups of (iv) as well as the heteroaryl groups of (iv) are optionally monosubstituted in the carbon skeleton by a fluorine, chlorine, or bromine atom, by a C₁₋₂-alkyl, trifluoromethyl, C₁₋₃-alkoxy, fluoromethoxy, difluoromethoxy, trifluoromethoxy, C₁₋₃-alkylamino, di-(C₁₋₃-alkyl)amino, amino-C₁₋₃-alkyl, acetylamino, acetyl, C₁₋₃-alkoxy-carbonyl-C₁₋₃-alkyl, C₁₋₅-alkoxy-carbonylamino-C₁₋₃-alkyl, C₁₋₃-alkylaminocarbonyl, di-(C₁₋₃-alkyl)aminocarbonyl, or cyano group or, with the exception of 5-membered heteroaryl groups containing more than two heteroatoms, they are optionally disubstituted by identical or different substituents selected from these substituents,
(v) a C₁₋₆-alkyl group substituted by a phenyl group and a carboxy, C₁₋₃-alkoxy-carbonyl, aminocarbonyl, C₁₋₃-alkylaminocarbonyl, or di-(C₁₋₃-alkyl)-aminocarbonyl group,
(vi) a phenyl-C₂₋₅-alkenylene-CH₂ or phenyl-C₂₋₅-alkynylene-CH₂ group, wherein a hydrogen atom of the methylene group in position 1 is optionally replaced by a methyl group and independently thereof the phenyl moiety thereof is optionally mono- or disubstituted by identical or different substituents selected from fluorine, chlorine, or bromine atom, by a C₁₋₄-alkyl, C₃₋₇-cycloalkyl, trifluoromethyl, C₁₋₃-alkoxy, phenyl, pyridyl, pyrimidinyl, pyrazinyl, thienyl, pyrrolyl, pyrazolyl, or thiazolyl group, wherein disubstitution by two aromatic groups is excluded,
(vii) R^{b}-A^{b}-E^{b}-C₁₋₃-alkyl optionally substituted in the C₁₋₃-alkyl moiety by a methyl group, wherein:
R^{b} is a phenyl group optionally mono- or disubstituted by fluorine, chlorine, or bromine atoms, by C₁₋₃-alkyl, cyclopropyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, acetylamino, acetyl, propionyl, carboxy, C₁₋₃-alkoxy-carbonyl, aminocarbonyl, C₁₋₃-alkylaminocarbonyl, di-(C₁₋₃-alkyl)aminocarbonyl, or cyano groups, wherein the substituents are identical or different, or
a 5-membered heteroaryl group which is optionally bound via a carbon atom or, if A^{b} is a bond, a —CH₂, —(CH₂)₂, sulfonyl, or carbonyl group, also is optionally bound via a nitrogen atom and which contains:
an imino group optionally substituted by a C₁₋₃-alkyl group, or an oxygen or sulfur atom,
an imino group optionally substituted by a C₁₋₃-alkyl group, or an oxygen or sulfur atom, and additionally a nitrogen atom,
an imino group optionally substituted by a C₁₋₃-alkyl group and two nitrogen atoms, or
an oxygen or sulfur atom and two nitrogen atoms,
a 6-membered heteroaryl group, which contains one or two nitrogen atoms,
wherein the heteroaryl groups of R^{b} are optionally monosubstituted in the carbon skeleton by a fluorine, chlorine, or bromine atom, by a C₁₋₄-alkyl, C₃₋₇-cycloalkyl, trifluoromethyl, phenyl, hydroxy, C₁₋₃-alkoxy, trifluoromethoxy, amino, C₁₋₃-alkylamino, di-(C₁₋₃-alkyl)-amino, acetylamino, acetyl, C₁₋₃-alkoxy-carbonyl, aminocarbonyl, C₁₋₃-alkylaminocarbonyl, or di-(C₁₋₃-alkyl)aminocarbonyl group or, with the exception of 5-membered heteroaryl groups containing more than two heteroatoms, they are optionally disubstituted by identical or different substituents selected from these substituents,
a C₃₋₇-cycloalkyl group, wherein:
one or two hydrogen atoms in each case are optionally replaced by a C₁₋₃-alkyl group and/or
in each case the methylene group in the 4 position of a cyclohexyl group is optionally replaced by an oxygen atom, by a sulfonyl group, or by an imino group optionally substituted by a C₁₋₃-alkyl, C₁₋₃-alkyl-carbonyl, C₁₋₃-alkoxy-carbonyl, C₁₋₃-alkylaminocarbonyl, or di-(C₁₋₃-alkyl)-aminocarbonyl group, or
the two hydrogen atoms of the methylene group in the 3 position of a cyclopentyl group or in 3 or 4 position of a cyclohexyl or cycloheptyl group is optionally replaced by an n-butylene, n-pentylene, n-hexylene, 1,2-ethylenedioxy, or 1,3-propylenedioxy group,
a 4- to 7-membered cycloalkyleneimino group, wherein:
the cycloalkylene moiety is optionally fused to a phenyl ring, or one or two hydrogen atoms in each case are optionally replaced by a C₁₋₃-alkyl group and/or
in each case the carbon atom in the 4 position of a 6- or 7-membered cycloalkyleneimino group is optionally substituted by 4- to 7-membered cycloalkyleneimino, phenyl, or 4-(C₁₋₃-alkyl)-1,2,4-triazol-3-yl, phenyl-C₁₋₃-alkylamino group, or is optionally replaced by an oxygen atom, by a sulfonyl group, or by an imino group optionally substituted by a C₁₋₃-alkyl, C₁₋₃-alkyl-carbonyl, C₁₋₃-alkylaminocarbonyl, or di-(C₁₋₃-alkyl)-aminocarbonyl group, or
the two hydrogen atoms of the methylene group in the 3 position of a 5-membered cycloalkyleneimino group or in the 3 or 4 position of a 6- or 7-membered cycloalkyleneimino group is optionally replaced by an n-butylene, n-pentylene, n-hexylene, 1,2-ethylenedioxy, or 1,3-propylenedioxy group, or
in a 5-, 6-, or 7-membered cycloalkyleneimino group, a —CH₂— group linked to the imino nitrogen atom is optionally replaced by a carbonyl group,
A^{b} is a bond, an oxygen atom, a —NH, —N(C₁₋₃-alkyl), sulfonyl, or a carbonyl group, or
—CH₂—, —(CH₂)₂—, —O—CH₂—, —CH₂—O—, NH—CH₂—, —CH₂—NH—, —NH—CO—, —CO—NH—, —NH—SO₂—, —SO₂—NH—, or —C≡C—, wherein a hydrogen atom bound to a carbon atom and/or a hydrogen atom bound to a nitrogen atom is optionally replaced by a C₁₋₃-alkyl group in each case and a heteroatom of A^{b} is not linked to a nitrogen atom of a 5-membered heteroaryl group of R^{b},
E^{b} is a phenylene group optionally substituted by a fluorine, chlorine, or bromine atom, or by a C₁₋₄-alkyl, trifluoromethyl, hydroxy, C₁₋₃-alkoxy, fluoromethoxy, difluoromethoxy, trifluoromethoxy, amino, C₁₋₃-alkylamino, di-(C₁₋₃-alkyl)amino, acetylamino, acetyl, carboxy, C₁₋₃-alkoxy-carbonyl, C₁₋₃-alkoxy-carbonyl-C₁₋₃-alkyl, aminocarbonyl, C₁₋₃-alkylaminocarbonyl, di-(C₁₋₃-alkyl)aminocarbonyl, or cyano group,
(viii) R^{c}-A^{c}-E^{c}-C₁₋₃-alkyl optionally substituted in the C₁₋₃-alkyl moiety by a C₁₋₄-alkyl or C₃₋₅-cycloalkyl group, wherein:
R^{c} is R^{b}, wherein any reference in R^{b} to A^{b} is replaced by reference to A^{c},
A^{c} is a bond, an oxygen atom, a —CH₂—, —NH—, —N(C₁₋₃-alkyl)-, —NH—CO—, —CO—NH—, or carbonyl group, wherein a heteroatom of A^{c} is not linked to a nitrogen atom of a 5-membered heteroaryl group of R^{c}, and
E^{c} is a 5-membered heteroarylene group bound via two carbon atoms or via a carbon atom and an imino-nitrogen atom, wherein the imino-nitrogen atom of the heteroarylene group is not linked to a heteroatom of A^{c} and the heteroarylene group contains:
an imino group optionally substituted by a C₁₋₃-alkyl group, or an oxygen or sulfur atom,
an imino group optionally substituted by a C₁₋₃-alkyl group, or an oxygen or sulfur atom, and additionally a nitrogen atom, or
an imino group optionally substituted by a C₁₋₃-alkyl group and two nitrogen atoms, or
an oxygen or sulfur atom and two nitrogen atoms,
or a 6-membered heteroarylene group, which contains one or two nitrogen atoms,
and wherein the 5- and 6-membered heteroarylene groups of E^{c} are optionally substituted in the carbon skeleton by a fluorine, chlorine, or bromine atom, or by a C₁₋₄-alkyl, C₃₋₇-cycloalkyl, trifluoromethyl, hydroxy, C₁₋₃-alkoxy, trifluoromethoxy, amino, C₁₋₃-alkylamino, acetylamino, acetyl, C₁₋₃-alkoxy-carbonyl, aminocarbonyl, C₁₋₃-alkylaminocarbonyl, or cyano group, or
R⁶ and R⁷ together are an n-alkylene bridge with 4 or carbon atoms, wherein:
one or two hydrogen atoms is optionally replaced by a C₁₋₃-alkyl group, and/or
a —CH₂—CH₂ group is optionally replaced by a 1,2-linked phenylene group optionally monosubstituted by a fluorine, chlorine, or bromine atom, by a C₁₋₃-alkyl, trifluoromethyl, hydroxy, C₁₋₃-alkoxy, trifluoromethoxy, amino, C₁₋₃-alkylamino, di-(C₁₋₃-alkyl)amino, acetylamino, acetyl, C₁₋₃-alkoxy-carbonyl, aminocarbonyl, C₁₋₃-alkylaminocarbonyl, cyano, phenyloxy, or phenyl-C₁₋₃-alkyl group, wherein the phenyloxy- and phenyl-C₁₋₃-alkyl group in the phenyl moiety are optionally substituted by a fluorine, chlorine, or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino, or cyano group,
or in each case the carbon atom in the 3 position of an n-pentylene group is optionally monosubstituted by a C₁₋₃-alkyl group terminally substituted by a phenyl, amino, C₁₋₃-alkylamino, di-(C₁₋₃-alkyl)-amino, or a 5- to 7-membered cycloalkyleneimino group, C₁₋₃-alkoxy-carbonyl, aminocarbonyl, C₁₋₃-alkylaminocarbonyl, or di-(C₁₋₃-alkyl)-amino-carbonyl group, or is optionally disubstituted by a phenyl group and a cyano group, or
the methylene group in the 3 position of an n-pentylene group is optionally replaced by an oxygen atom, by a sulfonyl group, or by an imino group optionally substituted by a C₁₋₃-alkyl or C₁₋₃-alkyl-carbonyl group,
the methylene group in the 3 position of an n-pentylene group is optionally replaced by an oxygen atom, by a sulfonyl group, or by an imino group optionally substituted by a C₁₋₃-alkyl or C₁₋₃-alkyl-carbonyl group; and
R⁸ is a hydrogen atom or a C₁₋₃-alkyl group,
wherein:
the unsubstituted or monosubstituted phenyl groups and the aromatic or heteroaromatic moieties are, unless otherwise stated, optionally additionally substituted in the carbon skeleton by identical or different substituents selected from fluorine, chlorine, or bromine atoms, C₁₋₃-alkyl, trifluoromethyl, hydroxy, C₁₋₃-alkoxy, trifluoromethoxy, phenyl, amino, C₁₋₃-alkylamino, acetylamino, acetyl, C₁₋₃-alkoxy-carbonyl, aminocarbonyl, C₁₋₃-alkylaminocarbonyl, or cyano groups, so that the resulting aromatic or heteroaromatic moieties are each optionally at most disubstituted,
the hydrogen atoms in the C₁₋₃-alkyl and alkoxy groups are optionally wholly or partially replaced by fluorine atoms, and
the alkyl and alkoxy groups and the alkyl moieties contained in formula I with more than two carbon atoms are each optionally straight-chain or branched, unless otherwise specified,
or a tautomer, enantiomer, diastereomer, or salt thereof.

3. The compound of formula I according to claim 2, wherein:
Het is a 2,4-linked pyrrolylene or imidazolylene group which are bound in each case via the 2 position to the adjacent carbonyl group of formula I and are substituted at a nitrogen atom by a C₁₋₃-alkyl group and is optionally substituted in the carbon skeleton by a C₁₋₃-alkyl group or a trifluoromethyl group,
R⁷ is a C₁₋₄-alkyl group terminally substituted by a C₃₋₇-cycloalkyl group, wherein:
a hydrogen atom in the 4 position of a cyclohexyl group is optionally replaced by a C₁₋₅-alkoxy, C₁₋₃-alkoxy-C₁₋₃-alkyl, phenyl-C₁₋₃-alkoxy-methyl, phenyl-C₁₋₃-alkylamino, phenyl-C₁₋₂-alkyl-carbonylamino, benzoylamino, phenylaminocarbonyl, phenyl-C₁₋₃-alkylaminocarbonyl, carboxy, or C₁₋₃-alkoxy-carbonyl group, or
in a cyclopentyl group one or two single bonds separated from each other and from position 1 by at least one bond are each optionally fused to a phenyl group, wherein in a bi-or tricyclic ring system thus formed, the hydrogen atom bound to the saturated carbon atom in the 1 position is optionally replaced by a C₁₋₃-alkylaminocarbonyl or di-(C₁₋₃-alkyl)aminocarbonyl group, wherein terminal methyl groups in each case are optionally wholly or partly fluorinated,
a C₁₋₆-alkyl group optionally substituted by a C₃₋₅-cycloalkyl group which is substituted by a phenyl, 1-naphthyl, 2-naphthyl, pyridinyl, pyrimidinyl, pyrrolyl, furanyl, thienyl, pyrazolyl, imidazolyl, oxazolyl, isoxazolyl, thiazolyl, or isothiazolyl group, wherein a nitrogen atom of the pyrrolyl, pyrazolyl and imidazolyl group is optionally substituted by a C₁₋₃-alkyl or trifluoromethyl group and the phenyl group and/or the heteroaromatic group thereof is optionally substituted in the carbon skeleton by a fluorine, chlorine, or bromine atom, by a C₁₋₄-alkyl, trifluoromethyl, C₁₋₃-alkoxy, fluoromethoxy, difluoromethoxy, trifluoromethoxy, C₁₋₄-alkoxy-carbonylamino-C₁₋₃-alkyl, amino, C₁₋₃-alkylamino, di-(C₁₋₃-alkyl)amino, or cyano group,
a C₁₋₆-alkyl group substituted by a phenyl group and a carboxy or C₁₋₃-alkoxy-carbonyl group,
a phenyl-C₂₋₃-alkynylene-CH₂ group wherein a hydrogen atom of the methylene group in the 1 position is optionally replaced by a methyl group and independently thereof the phenyl moiety is optionally substituted by a fluorine, chlorine, or bromine atom, or by a C₁₋₄-alkyl, trifluoromethyl, C₁₋₃-alkoxy, phenyl, or cyano group,
R^{b}-A^{b}-E^{b}-C₁₋₃-alkyl optionally substituted in the C₁₋₃-alkyl moiety by a methyl group, wherein:
R^{b} is a phenyl group optionally substituted by a fluorine, chlorine, or bromine atom, by a C₁₋₃-alkyl, trifluoromethyl, hydroxy, C₁₋₃-alkoxy, fluoromethoxy, difluoromethoxy, trifluoromethoxy, carboxy, or C₁₋₃-alkoxy-carbonyl group,
a 5-membered heteroaryl group which is optionally bound via a carbon atom or, if A^{b} is a bond, is optionally also bound via a nitrogen atom and contains:
an imino group optionally substituted by a C₁₋₃-alkyl group, or an oxygen or sulfur atom,
an imino group optionally substituted by a C₁₋₃-alkyl group, or an oxygen or sulfur atom, and additionally a nitrogen atom, or
an imino group optionally substituted by a C₁₋₃-alkyl group and two nitrogen atoms, or
an oxygen or sulfur atom and two nitrogen atoms,
a 6-membered heteroaryl group containing one or two nitrogen atoms,
wherein the heteroaryl group of R^{b} is optionally monosubstituted in the carbon skeleton by a fluorine, chlorine, or bromine atom, by a C₁₋₃-alkyl, trifluoromethyl, phenyl, C₁₋₃-alkoxy, trifluoromethoxy, amino, C₁₋₃-alkylamino, di-(C₁₋₃-alkyl)-amino, or acetylamino group or, with the exception of 5-membered heteroaryl groups containing more than two heteroatoms, is optionally also disubstituted by a C₁₋₄-alkyl group and one substituent selected from fluorine, chlorine, bromine, C₁₋₃-alkyl, trifluoromethyl, phenyl, C₁₋₃-alkoxy and trifluoromethoxy,
a C₃₋₆-cycloalkyl group, wherein the two hydrogen atoms of the methylene group in the 3 position of a cyclopentyl group, or in the 3 or 4 position of a cyclohexyl group are optionally replaced by an n-butylene, n-pentylene, or 1,2-ethylenedioxy group,
a 5- to 7-membered cycloalkyleneimino group, wherein:
the cycloalkylene moiety is optionally fused to a phenyl ring, or
a hydrogen atom is optionally replaced by a C₁₋₃-alkyl group and/or
in each case the carbon atom in the 4 position of a 6- or 7-membered cycloalkyleneimino group is optionally substituted by a 4- to 7-membered cycloalkyleneimino, phenyl, or 4-(C₁₋₃-alkyl)-1,2,4-triazol-3-yl group, or
the two hydrogen atoms of the methylene group in the 3 position of a 5-membered cycloalkyleneimino group or in the 3 or 4 position of a 6- or 7-membered cycloalkyleneimino group is optionally replaced by an n-butylene, n-pentylene, or 1,2-ethylenedioxy group,
A^{b} is a bond, an oxygen atom, —CH₂—, —NH—, —O—CH₂—, carbonyl, —NH—CO—, or —CO—NH— group,
wherein a hydrogen atom bound to a nitrogen atom is optionally replaced in each case by a C₁₋₃-alkyl group,
E^{b} is a phenylene group optionally substituted by a fluorine, chlorine, or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino, or C₁₋₃-alkoxy-carbonyl group, or
R^{c}-A^{c}-E^{c}-C₁₋₃-alkyl, wherein:
R^{c} is a phenyl group optionally substituted by a fluorine, chlorine, or bromine atom, by a C₁₋₃-alkyl, trifluoromethyl, C₁₋₃-alkoxy, trifluoromethoxy, carboxy, or C₁₋₃-alkoxy-carbonyl group, or
a 5- to 7-membered cycloalkyleneimino group, wherein:
the cycloalkylene moiety is optionally fused to a phenyl ring, or
a hydrogen atom is optionally replaced by a C₁₋₃-alkyl group, and/or
the two hydrogen atoms of the methylene group in the 3 position of a 5-membered cycloalkyleneimino group or in the 3 or 4 position of a 6- or 7-membered cycloalkyleneimino group are optionally replaced by an n-butylene, n-pentylene, or 1,2-ethylenedioxy group,
A^{c} is a bond,
E^{c} is a 5-membered heteroarylene group bound via two carbon atoms which contains:
an imino group optionally substituted by a C₁₋₃-alkyl group, or an oxygen or sulfur atom,
an imino group optionally substituted by a C₁₋₃-alkyl group, or an oxygen or sulfur atom, and additionally a nitrogen atom, or
an imino group optionally substituted by a C₁₋₃-alkyl group and two nitrogen atoms, or
an oxygen or sulfur atom and two nitrogen atoms,
or a pyridinylene, pyridazinylene, or pyrimidinylene group,
wherein the 5- and 6-membered heteroarylene groups of EC are optionally substituted in the carbon skeleton by a fluorine, chlorine, or bromine atom, by a C₁₋₃-alkyl, trifluoromethyl, C₁₋₃-alkoxy, trifluoromethoxy, amino, C₁₋₃-alkylamino, acetylamino, C₁₋₃-alkoxy-carbonyl, or cyano group,
or R⁶ and R⁷ together denote an n-alkylene bridge with 4 or 5 carbon atoms, wherein:
a hydrogen atom is optionally replaced by a C₁₋₃-alkyl group, and/or
a —CH₂—CH₂— group is optionally replaced by a 1,2-linked phenylene group optionally substituted by a phenyloxy or benzyl group, wherein the phenyloxy or benzyl group in the aromatic moiety and the phenylene group is optionally substituted independently of one another by a fluorine, chlorine, or bromine atom, by a C₁₋₃-alkyl, trifluoromethyl, C₁₋₃-alkoxy, trifluoromethoxy, amino, C₁₋₃-alkylamino, di-(C₁₋₃-alkyl)amino, acetylamino, C₁₋₃-alkoxy-carbonyl, or cyano group,
or the carbon atom in the 3 position of an n-pentylene group is optionally monosubstituted by a C₁₋₃-alkyl group terminally substituted by an amino, C₁₋₃-alkylamino, di-(C₁₋₃-alkyl)-amino, acetylamino, or N-(methyl)-acetylamino group, or a 5- to 7-membered cycloalkyleneimino group, or is optionally disubstituted by a phenyl group and a cyano group,
wherein:
the unsubstituted or monosubstituted phenyl groups and the aromatic or heteroaromatic moieties are, unless otherwise stated, optionally additionally substituted in the carbon skeleton by identical or different substituents selected from fluorine, chlorine, or bromine atoms, C₁₋₃-alkyl groups, trifluoromethyl, C₁₋₃-alkoxy, trifluoromethoxy, phenyl, amino, C₁₋₃-alkylamino, acetylamino, acetyl, C₁₋₃-alkoxy-carbonyl, aminocarbonyl, C₁₋₃-alkylaminocarbonyl, or cyano groups, so that the resulting aromatic or heteroaromatic moieties are each optionally at most disubstituted,
the alkyl and alkoxy groups and the alkyl moieties contained in formula I with more than two carbon atoms are each optionally straight-chain or branched, unless otherwise specified,
the carboxy groups mentioned in the definition of the abovementioned groups are optionally replaced by a group which can be converted into a carboxy group in vivo or by a group which is negatively charged under physiological conditions, and/or
the amino and imino groups mentioned in the definition of the abovementioned groups is optionally substituted by a group which can be cleaved in vivo,
or a tautomer, enantiomer, diastereomer, or salt thereof.

4. The compound of formula I according to claim 3, wherein the unsubstituted or monosubstituted phenyl groups and the aromatic or heteroaromatic moieties are, unless otherwise stated, optionally additionally substituted in the carbon skeleton by identical or different substituents selected from fluorine, chlorine, or bromine atoms, C₁₋₃-alkyl groups, trifluoromethyl, C₁₋₃-alkoxy, trifluoromethoxy, phenyl, amino, C₁₋₃-alkylamino, acetylamino, C₁₋₃-alkoxy-carbonyl, or cyano groups, so that the resulting aromatic or heteroaromatic moieties are each optionally at most disubstituted, or a tautomer, enantiomer, diastereomer, or salt thereof.

5. The compound of formula I according to claim 1, wherein:
X₁ is CR;
X₂ is CR²;
X₃ is CR³; and
X₄ is CR⁴,
wherein R¹, R², R³, and R⁴ are each a hydrogen atom, or one of R¹ to R⁴ is a fluorine, chlorine, or bromine atom, a C₁₋₃-alkyl group, or a trifluoromethyl group and the remainder of R¹ to R⁴ in each case are each a hydrogen atom;
A^{a} is a bond, an oxygen atom, or a —CH₂—, —(CH₂)₂—, —NH—, —N(C₁₋₃-alkyl)-group, wherein a nitrogen atom of A^{a} is not linked to a nitrogen atom of a 5-membered heteroaryl group of R^{a};
R^{a} is a phenyl, 2-pyridinyl, 3-pyridinyl, 4-pyridinyl group, 1-pyrrolyl, 2-pyrrolyl, 3-pyrrolyl, 2-thienyl, or 3-thienyl group, wherein the nitrogen atom of the pyrrolyl group is optionally substituted by a C₁₋₃-alkyl group and the phenyl group and the heteroaromatic group thereof are optionally substituted in the carbon skeleton by a fluorine, chlorine, or bromine atom, by a C₁₋₃-alkyl or trifluoromethyl group, or
a pyrrolidino, piperidino, or morpholino group;
R⁵ is a hydrogen atom;
Het is a 2,4-linked pyrrolylene or imidazolylene group which are bound in each case via the 2 position to the adjacent carbonyl group of formula I and are substituted at a nitrogen atom by a C₁₋₃-alkyl group and is optionally substituted in the carbon skeleton by a C₁₋₃-alkyl group or a trifluoromethyl group;
R⁶ is a hydrogen atom; and
R⁷ is (i) R^{d}—CH₂— or R^{d}—CH₂—CH₂—, wherein a hydrogen atom of the methylene group is optionally replaced in the 1 position by a C₁₋₃-alkyl group or a cyclopropyl group and wherein R^{d} is a phenyl, 1-naphthyl, 2-naphthyl, 2-pyridinyl, 3-pyridinyl, 4-pyridinyl, 2-pyrimidinyl, or 5-pyrimidinyl group, wherein the phenyl group and the heteroaromatic groups thereof are optionally substituted in the carbon skeleton by a fluorine, chlorine, or bromine atom, or by a C₁₋₄-alkyl, trifluoromethyl, C₁₋₃-alkoxy, or fluoromethoxy group,
(ii) phenyl-C≡C—CH₂—, wherein a hydrogen atom of the methylene group in the 1 position is optionally replaced by a methyl group and independently thereof the phenyl moiety is optionally substituted by a fluorine, chlorine, or bromine atom, or by a C₁₋₄-alkyl, trifluoromethyl, or phenyl group,
(iii) R^{b}-A^{b}-E^{b}-CH₂—, wherein a hydrogen atom of the methylene group is optionally replaced in the 1 position by a methyl group, wherein:
R^{b} is a phenyl group optionally substituted by fluorine, chlorine, or bromine atoms, or by C₁₋₃-alkyl, trifluoromethyl, hydroxy, methoxy, carboxy, or methoxycarbonyl group, or
a pyrrolyl, pyrazolyl, imidazolyl, oxazolyl, isoxazolyl, thiazolyl, isothiazolyl, oxadiazolyl, or thiadiazolyl group which is optionally bound via a carbon atom or, if A^{b} is a bond, also is bound via a nitrogen atom,
a 2-pyridyl, 3-pyridyl, 4-pyridyl, pyrazinyl, 2-pyrimidinyl, 4-pyrimidinyl, 5-pyrimidinyl, 3-pyridazinyl, or 4-pyridazinyl group,
wherein a phenyl ring is optionally fused to the 5- or 6-membered heteroaryl groups of R^{b} via two adjacent carbon atoms and the bicyclic heteroaryl groups thus formed is optionally bound via the heteroaromatic or carbocyclic moiety,
wherein the mono- and bicyclic heteroaryl groups of R^{b} are optionally monosubstituted in the carbon skeleton by a fluorine, chlorine, or bromine atom, by a C₁₋₃-alkyl, trifluoromethyl, phenyl, amino, C₁₋₃-alkylamino, di-(C₁₋₃-alkyl)-amino, or acetylamino group or, with the exception of 5-membered heteroaryl groups containing more than two heteroatoms, they are optionally disubstituted by identical or different substituents selected from fluorine, chlorine, bromine, C₁₋₃-alkyl, trifluoromethyl, or phenyl,
a C₅₋₆-cycloalkyl group, wherein the two hydrogen atoms of the methylene group in the 3 position of a cyclopentyl group or in the 4 position of the cyclohexyl group is optionally replaced by an n-butylene, n-pentylene, or 1,2-ethylenedioxy group,
a 5- to 7-membered cycloalkyleneimino group, wherein:
the cycloalkylene moiety is optionally fused to a phenyl ring optionally substituted by a fluorine, chlorine, or bromine atom, by a C₁₋₃-alkyl, trifluoromethyl, or C₁₋₃-alkoxy group, or
a hydrogen atom is optionally replaced by a C₁₋₃-alkyl group, and/or
the two hydrogen atoms of the methylene group in the 3 position of the 5-membered cycloalkyleneimino group or in the 4 position of the 6-membered cycloalkyleneimino group is optionally replaced by an n-butylene, n-pentylene, or 1,2-ethylenedioxy group,
A^{b} is a bond, —CH₂—, —NH—, —O—CH₂—, —NH—CO—, or —CO—NH—, wherein a hydrogen atom bound to a nitrogen atom is optionally replaced by a methyl group in each case,
E^{b} is a 1,4-linked phenylene group optionally substituted by a fluorine, chlorine, or bromine atom, or by a C₁₋₃-alkyl, trifluoromethyl, C₁₋₃-alkoxy, or trifluoromethoxy group,
(iv) R^{c}-A^{c}-E^{c}-C₁₋₃-alkyl, wherein:
R^{c} is a phenyl group optionally substituted by a fluorine, chlorine, or bromine atom, or by a C₁₋₃-alkyl, trifluoromethyl, methoxy, carboxy, or methoxycarbonyl group,
A^{c} is a bond,
E^{c} is a pyrrolylene, pyrazolylene, imidazolylene, oxazolylene, isoxazolylene, thiazolylene, isothiazolylene, [1,3,4]-oxadiazolene, or [1,3,4]-thiadiazolene group bound via two carbon atoms in the relative positions 1,3, wherein a hydrogen atom bound to a nitrogen atom is optionally replaced by a C₁₋₃-alkyl group, or
a 1,4-linked pyridinylene, pyridazinylene, or pyrimidinylene group,
wherein the 5- and 6-membered heteroarylene groups of EC are optionally substituted in the carbon skeleton by a fluorine, chlorine, or bromine atom, or by a C₁₋₃-alkyl, trifluoromethyl, or methoxy group, or
wherein:
the alkyl and alkoxy groups and the alkyl moieties contained in formula I with more than two carbon atoms are each optionally straight-chain or branched, unless otherwise specified,
the carboxy groups are optionally replaced by a group convertible into a carboxy group in vivo or by a group which is negatively charged under physiological conditions, and
the amino and imino groups are optionally substituted by a group which can be cleaved in vivo,
or a tautomer, enantiomer, diastereomer, or salt thereof.

6. A compound selected from:
(a) N-[3-(biphenyl-4-yl)-prop-2-ynyl]-4-(4′-trifluoromethylbiphenyl-2-carbonylamino)-1-methylpyrrole-2-carboxylic acid amide;
(b) N-[4-(1,4-dioxa-8-azaspiro[4.5]dec-8-yl)-phenylmethyl]-4-(4′-trifluoromethylbiphenyl-2-carbonylamino)-1-methylpyrrole-2-carboxylic acid amide;
(c) N-[4-(1,4-dioxa-8-azaspiro[4.5]dec-8-yl)-phenylmethyl]-4-(4′-trifluoromethylbiphenyl-2-carbonylamino)-1-methylpyrrole-2-carboxylic acid amide;
(d) N-[4-(6-methylpyridazin-3-yl)-phenylmethyl]-4-(4′-trifluoromethylbiphenyl-2-carbonylamino)-1-methylpyrrole-2-carboxylic acid amide;
(e) N-(4′-hydroxybiphenyl-4-yl)methyl-4-(4′-trifluoromethylbiphenyl-2-carbonylamino)-1-methylpyrrole-2-carboxylic acid amide;
(f) N-[4-(1,4-dioxaspiro[4.5]dec-8-yl)-phenylmethyl]-4-(4′-trifluoromethylbiphenyl-2-carbonylamino)-1-methylpyrrole-2-carboxylic acid amide;
(g) N-(4′-methylbiphenyl-4-yl)methyl-4-(4′-trifluoromethylbiphenyl-2-carbonylamino)-1-methylpyrrole-2-carboxylic acid amide;
(h) N-[3-(4-isopropylphenyl)-prop-2-ynyl]-4-(4′-trifluoromethylbiphenyl-2-carbonylamino)-1-methylpyrrole-2-carboxylic acid amide;
(i) N-[3-(4-biphenyl)-prop-2-ynyl]-4-(4′-trifluoromethylbiphenyl-2-carbonylamino)-1-methylimidazole-2-carboxylic acid amide; and
(j) N-[3-(4-trifluoromethylphenyl)-prop-2-ynyl]-4-(4′-trifluoromethylbiphenyl-2-carbonylamino)-1-methylimidazole-2-carboxylic acid amide,
and the salts thereof.

7. A physiologically acceptable salt of the compound according to claim 1.

8. A physiologically acceptable salt of the compound according to claim 2.

9. A physiologically acceptable salt of the compound according to claim 3.

10. A physiologically acceptable salt of the compound according to claim 4.

11. A physiologically acceptable salt of the compound according to claim 5.

12. A physiologically acceptable salt of the compound according to claim 6.

13. A pharmaceutical composition comprising a compound according to claim 1 and a pharmaceutically acceptable carrier or diluent.

14. A pharmaceutical composition comprising a compound according to claim 2 and a pharmaceutically acceptable carrier or diluent.

15. A pharmaceutical composition comprising a compound according to claim 3 and a pharmaceutically acceptable carrier or diluent.

16. A pharmaceutical composition comprising a compound according to claim 4 and a pharmaceutically acceptable carrier or diluent.

17. A pharmaceutical composition comprising a compound according to claim 5 and a pharmaceutically acceptable carrier or diluent.

18. A pharmaceutical composition comprising a compound according to claim 6 and a pharmaceutically acceptable carrier or diluent.

19. A pharmaceutical composition comprising a physiologically acceptable salt according to claim 7 and a pharmaceutically acceptable carrier or diluent.

20. A pharmaceutical composition comprising a physiologically acceptable salt according to claim 8 and a pharmaceutically acceptable carrier or diluent.

21. A pharmaceutical composition comprising a physiologically acceptable salt according to claim 9 and a pharmaceutically acceptable carrier or diluent.

22. A pharmaceutical composition comprising a physiologically acceptable salt according to claim 10 and a pharmaceutically acceptable carrier or diluent.

23. A pharmaceutical composition comprising a physiologically acceptable salt according to claim 11 and a pharmaceutically acceptable carrier or diluent.

24. A pharmaceutical composition comprising a physiologically acceptable salt according to claim 12 and a pharmaceutically acceptable carrier or diluent.
